SEQUENCE LISTING (216 sequences)

SEQ ID NO: 1 - DNA, 501 bp, artificial sequence
  control nucleic acid sequence
aagtgccgcgttgtagaaatgagcgcaacctctgcaagaggacggtctgagattagggatcgtactacaacgggttgtgtattcgtcgaggtgactgtcgtaccgcttgagtcgtaagaagtgagtgttagattttcgaataatgtgttggtcgagactaacggaggcgcctggcgcagaaactgcactcaattcgattcctactgtagccgttggtgctcgacggtgaatgatgtaggtaccagcagtaactagagcacgtcttcgaccaaatctggatattgcagcctcgtcgtagcctcgcaccttcatttcggaggatcgtcgagttcgtctgggtgtcctactacaactgggtgtagcagcctaacactgtcattgcggtcgagccaactcgtacctgactttgtgtaaattgcctcggcttaaacagggaaacgtcttctactaggacaacgtacacttgacgcgccgtttgaataatggtcctcctcggcccag

SEQ ID NO: 2 - DNA, 528 bp, artificial sequence
  control nucleic acid sequence
tttttttttttttttttttttttttttctgggccgaggaggaccattattcaaacggcgcgtcaagtgtacgttgtcctagtagaagacgtttccctgtttaagtcgaggcaatttacacaaagtcaggtacgagttggctcgaccgcaatgacagtgttaggctgctacacccagttgtagtaggacacccagacgaactcgacgatcctccgaaatgaaggtgcgaggctacgacgaggctgcaatatccagatttggtcgaagacgtgctctagttactgctggtacctacatcattcaccgtcgagcaccaacggctacagtaggaatcgaattgagtgcagtttctgcgccaggcgcctccgttagtctcgaccaacacattattcgaaaatctaacactcacttcttacgactcaagcggtacgacagtcacctcgacgaatacacaacccgttgtagtacgatccctaatctcagaccgtcctcttgcagaggttgcgctcatttctacaacgcggcactt

SEQ ID NO: 3 - DNA, 500 bp, artificial sequence
  control nucleic acid sequence
gcgttacagcctcaccccctgttgattaccgtacctcttctagcttgtcaagtataatcaacaggtgagtccaggcctggtacgatcatcgtctcggctgagttaacggacgtgaccgaagtacacgacgacgatcgaaagaaacttgccgcactagcgggtgtcgtagtggtattgtgcggggctagtgtatgtctagcgacggcaaaagaaagtgtttgacttgcaatatagggaactttggaataggaaccaaagttgcggctcagcgctcatagagacactagagttgatacgactaataagcttgacccgaattatcacgacgagatcatatagtccaccctcagctagggtatgcattctgcatctgacatacaacccttccgctacccctactcatatcaagtgttatgagggcaattcgcagccatactcagatttcgcccgcttgggtggtgatgaccgtaagtcgaggcacgtgccctgtgaaagctcaa

SEQ ID NO: 4 - DNA, 525 bp, artificial sequence
  control nucleic acid sequence
tttttttttttttttttttttttttttgagctttcacagggcacgtgcctcgacttacggtcatcaccacccaagcgggcgaaatctgagtatggctgcgaattgccctcataacacttgatatgagtaggggtagcggaagggttgtatgtcagatgcagaatgcataccctagctgagggtggactatatgatctcgtcgtgataattcgggtcaagcttattagtcgtatcaactctagtgtctctatgagcgctgagccgcaactttggttcctattccaaagttccctatattgcaagtcaaacactttcttttgccgtcgctagacatacactagccccgcacaataccactacgacacccgctagtgcggcaagtttctttcgatcgtcgtcgtgtacttcggtcacgtccgttaactcagccgagacgatgatcgtaccaggcctggactcacctgttgattatacttgacaagctagaagaggtacggtaatcaacagggggtgaggctgtaacgc

SEQ ID NO: 5 - DNA, 500 bp, artificial sequence
  control nucleic acid sequence
  misc_feature (13)..(13): n = any nucleotide
  misc_feature (14)..(14): n = any nucleotide
  misc_feature (17)..(17): n = any nucleotide
aaaactgtgagcnnttntcaaaatcaaactcgacatgtgcacgatatggtttcaaaagaacggggtgaatgctgaaggctgttcctagtgcgtatccacttcacgtgttcagttgcgcttgactgttgatagaaactcgtcaaccccgcaaccaggaccccgagcccaaaatacgagtcgtatatagtgtccagtctgaggtgtttactcgacacatcggcagttatggccatataatggttggagccaatcatttacattgtctgaggcggacgcacatcttaagaaacgaccagacatccggactggagtgtttgtaccttcaatattttaacatgaccccgggtcggatgatgggtgcggttgctacctcgatgtgtcgagtatatgggggtcgagcacatcaggcgcctcgttcggtgtggtcgcgttcttgttatatcatggactacaagtctgtgcggtctgggtcgctgtgactacgacttgtgatggctccg

SEQ ID NO: 6 - DNA, 525 bp, artificial sequence
  control nucleic acid sequence
tttttttttttttttttttttttttcggagccatcacaagtcgtagtcacagcgacccagaccgcacagacttgtagtccatgatataacaagaacgcgaccacaccgaacgaggcgcctgatgtgctcgacccccatatactcgacacatcgaggtagcatccgcacccatcatccgacccggggtcatgttaaaatattgaaggtacaaacactccagtccggatgtctggtcgtttcttaagatgtgcgtccgcctcagacaatgtaaatgattggctccaaccattatatggccataactgccgatgtgtcgagtaaacacctcagactggacactatatacgactcgtattttgggctcggggtcctggttgcggggttgacgagtatctatcaacagtcaagcgcaactgaacacgtgaagtggagacgcactaggaacagccttcagcattcaccccgttcttttgaaaccatatcgtgcacatgtcgagtttgattttgagacgtgctcacagtttt

SEQ ID NO: 7 - DNA, 501 bp, artificial sequence
  control nucleic acid sequence
aatgacggttacgagaacaacatttgcccagagttcgttcaccatcagatcgtacaactaggcggtacggcttttttataagacacaattctgctttggtgggtcgggaagtatatcagcactttcggggtacaatatcagaccgccgacgactaaccagctagacaaggactattggtcacttactcgggtctcctgggcccctcactttctctgctagccacactgttatgaggcggtcgagggaatcacgccaacacaaccgcacgaatggaggccgtcaaaaggcaggcaagtgtaagctcaccccggcgattgtagggacaaacttttcgttgaggtccgggtcaatacttcactcgtcatgaatgatagaggggggcgacagtccagcaattccaagagggttttactattctatgcactcacctgcgccaccgctcgatcggtgcctgtgaataagaaatcgatctttgggaaattcatagtatggtgcactga

SEQ ID NO: 8 - DNA, 525 bp, artificial sequence
  control nucleic acid sequence
ttttttttttttttttttttttttttcagtgcaccatactatgaatttcccaaagatcgatttcttattcacaggcaccgatcgagcggtggcgcaggtgagtgcatagaatagtaaaaccctcttggaattgctggactgtcgcccccctctatcattcatgacgagtgaagtattgaccggacctcaacgaaaagtttgtccctacaatcgccggggtgagcttacacttgcctgccttttgacggcctccattcgtgcggttgtgttggcgtgattccctcgaccgcctcataacagtgtggctagcagagaaagtgaggggcccaggagacccgagtaagtgaccaatagtccttgtctagctggttagtcgtcggcggtctgatattgtaccccgaaagtgctgatatacttcccgacccaccaaagcagaattgtgtcttataaaaaagccgtaccgcctagttgtacgatctgatggtgaacgaactctgggcaaatgttgttctcgtaaccgtcatt

SEQ ID NO: 9 - DNA, 500 bp, artificial sequence
  control nucleic acid sequence
gagatattgtacactaaaccaaatggacgagtcaaatgctctcgcaactcgcagttaattagagacagtaagtcgttcgaagaatggcgctacgacaacaaacgaagtcgtggacttgtgctgctcaattgtgttgatcactgtggtatggccctgggacgcacatgcacagttttgactggaccgtgatgggtcacatgcgtagtcaggtctgaacccactgccaggagcgtcctcacgcctatgtgtcgagtaaccatagttttgaggcgtacgccgagcatacacctgtctcttggtaggctcatcgacggaatgcaaagctctgatcggcccgagctcgcaaaggctggcgccttttgggattataaaccgagtccgctgatgtgaccacctacttgaacaacccaatgaatcgctttcatctaatgtaggacggagaattgtaaggtagggcaagagaaattacgatcctattgccggttcgtaaggcccgtgca

SEQ ID NO: 10 - DNA, 527 bp, artificial sequence
  control nucleic acid sequence
ttttttttttttttttttttttttttttgcacgggccttacgaaccggcaataggatcgtaatttctcttgccctaccttacaattctccgtcctacattagatgaaagcgattcattgggttgttcaagtaggtggtcacatcagcggactcggtttataatcccaaaaggcgccagcctttgcgagctcgggccgatcagagctttgcattccgtcgataagcctaccaagagacaggtgtatgctcggcgtacgcctcaaaactatggttactcgacacataggcgtgaggacgctcctggcagtgggttcagacctgactacgcatgtgacccatcacggtccagtcaaaactgtgcatgtgcgtcccagggccataccacagtgatcaacacaattgagcagcacaagtccacgacttcgtttgttgtcgtagcgccattcttcgaacgacttactgtctctaattaactgcgagttgcgagagcatttgactcgtccatttggtttagtgtacaatatctc

SEQ ID NO: 11 - DNA, 499 bp, artificial sequence
  control nucleic acid sequence
tttagtcaggagtgagaagaaccaggcttgtcctcataccgcgtggaaggatgaactgtgactggcccttcgggtacgagcttgatggagtttgcaagtgttagctatgcagggccgactccggcctcaatcgtgacacagcaaagatggtcaaactaatggtgtacttacccaagtttacggcagtcaacgtagttctggagcaaattaacccagctttctcaaggcaagggactgtggtggtgaaaagtttttatcttcatggggcactatcagctatcggagtggataaactagtggcgagagcagaatccccccacagatcgacaccgagcaggtagccacctgaggagtgtacctcagatatgtgcaataattgtggcgcctttgattgttgctatagagagtctagtagtgtgtgacgcgttgttgtgtaacaccttcttacttgctactgatttgtgacggccgcgagcccactactcccccgccgagattc

SEQ ID NO: 12 - DNA, 525 bp, artificial sequence
  control nucleic acid sequence
tttttttttttttttttttttttttgaatctcggcgggggagtagtgggctcgcggccgtcacaaatcagtagcaagtaagaaggtgttacacaacaacgcgtcacacactactagactctctatagcaacaatcaaaggcgccacaattattgcacatatctgaggtacactcctcaggtggctacctgctcggtgtcgatctgtggggggattctgctctcgccactagtttatccactccgatagctgatagtgccccatgaagataaaaacttttcaccaccacagtcccattgccttgagaaagctgggttaatttgctccagaactacgttgactgccgtaaacttgggtaagtacaccattagtttgaccatctttgctgtgtcacgattgaggccggagtcggccctgcatagctaacacttgcaaactccatcaagctcgtacccgaagggccagtcacagttcatccttccacgcggtatgaggacaagcctggttcttctcactcctgactaaa

SEQ ID NO: 13 - DNA, 500 bp, artificial sequence
  control nucleic acid sequence
tccagagagacgatccgcggagcgctgctctgttccttccgtcctcaaagcctcacacgctcgtccctgttaactcagtgtcagtgaaacctggtagcctctgattttgggaaacactgacccaagttactagcagatcacccggtggaaatttcactgttgagtgaccacatctacattgatggcatcatgactccaatcagttagaaacagtggcttgcgatataagcgtatccacgcggcacagctcgggttcgtgctgactttcgccgaccgatgtgtacttgtggtcacgacgacccttacaggtcgtatctaagactacgttacagacgcagtaactcagagattcaatggctcgctccatttaagtgacgtagaccgatagaacgacagggtatggttgtgaaatcgaacaacttagcgttaccttctccttacgatagactgaggacgtcgaatttcgtctgcttcggagcttactgccgtggttatccgta

SEQ ID NO: 14 - DNA, 525 bp, artificial sequence
  control nucleic acid sequence
ttttttttttttttttttttttttttacggataaccacggcagtaagctccgaagcagacgaaattcgacgtcctcagtctatcgtaggagaaggtaacgctaagttgttcgatttcacaaccataccctgtcgttctatcggtctacgtcacttaaatggagcgagccattgaatctctgagttactgcgtctgtaacgtagtcttagatacgacaatgtaagggtcgtcgtgaccacaagtacacatcggtcggcgaaagtcagcacgaacccgagctgtgccgcgtggatacgcttatatcgcaagccactgtttctaactgattggagtcatgatgccatcaatgtagatgtggtcactcaacagtgaaatttccaccgggtgatctgctagtaacttgggtcagtgtttcccaaaatcagaggctaccaggtttcactgacactgagttaacagggacgagcgtgtgaggctttgaggacggaaggaacagagcagcgctccgcggatcgtctctctgga

SEQ ID NO: 15 - DNA, 521 bp, artificial sequence
  control nucleic acid sequence
gaagtcctccaaccagaagaactgtgacccccccactcataacgactcacaacgattagctgaccaatttattcagctccaacggagtagtgtctgataacaagacgcttagctctgaccgagagggacgtgctagattaataatactaggctcggtctcaccaccagaccagcatggtcacagtctcattgcgcatggtcacagtctcattgctcgtcacaactaagtgggagctagggagccgacggctacggagtactaggtaaaggagaataatcttaagcaatgggcagtttcctctgattaattgtcagactccacgaactgacatgagtcgacgacggctagattttggttccgtgcgactccaagccggagtcagaccgaaccccatcatagaggatacagcgccattagactcttgtcgagactgacgctctacaaatgcgatctggacactttcagagcgtgattctgcactatctcgacggtccgcatcgacgcgggaagagtacataca

SEQ ID NO: 16 - DNA, 524 bp, artificial sequence
  control nucleic acid sequence
tttttttttttttttttttttttttgtatgtactcttcccgcgtcgatgcggaccgtcgagatagtgcagaatcacgctctgaaagtgtccagatcgcatttgtagagcgtcagtctcgacaagagtctaatggcgctgtatcctctatgatggggttcggtctgactccggcttggagtcgtacggaaccaaaatctagccgtcgtcgactcatgtcagttcgtggagtctgacaattaatcagaggaaactgcccattgcttaagattattctcctttacctagtactccgtagccgtcggctccctagctcccacttagttgtgacgagcaatgagactgtgaccatgctggtctggtggtgagaccgagcctagtattattaatctagcacgtccctctcggtcagagctaagcgtcttgttatcagacactactccgttggagctgaataaattggtcagctaatcgttgtgagtcgttatgagtgggtgggtcacagttcttctggttggaggacttc

SEQ ID NO: 17 - DNA, 500 bp, artificial sequence
  control nucleic acid sequence
tttcttaagccgtaattactttaactcactatacatttcccgaaaccatctgccaatgttcttggttaacagtatgtgtcacaaacgtaccagctctgcctaaatccggccaagtcgctttagcacctcatgtgagccgtgccgtccccaagtctagtgaccgttaactgttttccagaccctccgaatatcgtccctcgaccgggtgaccactgcgaaggacgctacgcagctgcgagtcttgaatgatttgtactgtaacgatcctcccacccagactcttgtgaagtgatgcgtcacacggtgatcatgttggacctcttaagccagctctgtggtctcgctgcaaggcggcatatcgtggccatgcacgctcttgttaagtggcactctacgtggaggggtgtgcctctaacctgaccgaaatagatccgaatttagtgttgctctacctcacacgatggccactgtaaccaccttcattcgacctcgcggttcat

SEQ ID NO: 18 - DNA, 525 bp, artificial sequence
  control nucleic acid sequence
tttttttttttttttttttttttttatgaaccgcgaggtcgaatgaaggtggttacagtggccatcgtgtgaggtagagcaacactaaattcggatctatttcggtcaggttagaggcacacccctccacgtagagtgccacttaacaagagcgtgcatggccacgatatgccgccttgcagcgagaccacagagctggcttaagaggtccaacatgatcaccgtgtgacgcatcacttcacaagagtctgggtgggaggatcgttacagtacaaatcattcaagactcgcagctgcgtagcgtccttcgcagtggtcacccggtcgagggacgatattcggagggtctggaaaacagttaacggtcactagacttggggacggcacggctcacatgaggtgctaaagcgacttggccggatttaggcagagctggtacgtttgtgacacatactgttaaccaagaacattggcagatggtttcgggaaatgtatagtgagttaaagtaattacggcttaagaaa

SEQ ID NO: 19 - DNA, 500 bp, artificial sequence
  control nucleic acid sequence
aggcgcagagtctgccctgttttcaactggatcatgtcaggacggtcgggattagagtatccgcttactcttcggatgcatagtcgagtccctatccgcccgcctgtaatttcccaatttgatacattcaaatgcctccgaatcaggaacatgcgtcctctaagaactttaggtgaccatcagcgtagcataccaactccttgactatactgcaatccaattcgctgtaacgtaccgagcttccaacgtttcatagtaattgaatcaagaagtcggaacgtctcttcgaacacactgattagacgagtatttacggtagtactgctactccttaaccgttctagagagggcggaaactgaccgtccctttcggtactcgacgtaacatgacgtctggaacgcctgcttgcccatcttcgctttcctgtacgggctgaatcagtaggcgtaccgggtctgaagttatgaaagagttgctctctggtccgcgttgcttccgt

SEQ ID NO: 20 - DNA, 525 bp, artificial sequence
  control nucleic acid sequence
tttttttttttttttttttttttttacggaagcaacgcggaccagagagcaactctttcataacttcagacccggtacgcctaccgattcagcccgtacgaggaaagcgaagatgggcaagcaggcgttccagacgtcatgttacgtcgagtaccgaaagggacggtcagtttccgccctctctagaacggttaaggagtagcagtactaccgtaaatactcgtctaatcagtgtgttcgaagagacgttccgacttcttgattcaattactatgaaacgttggaagctcggtacgttacagcgaattggattgcagtatagtcaaggagttggtatgctacgctgatggtcacctaaagttcttagaggacgcatgttcctgattcgggggcatttgaatgtatcaaattgggaaattacaggcgggcggatagggactcgactatgcatccgagagtaagcggatactctaatcccgaccgtcctgacatgatccagttgaaaacagggcagactctgcgcct

SEQ ID NO: 21 - DNA, 70 bp, artificial sequence
  primer
ggtgctcgacggtgaatgatgtaggtaccagcagtaactagagcacgtcttcgaccaaatctggatattg

SEQ ID NO: 22 - DNA, 70 bp, artificial sequence
  primer
caatatccagatttggtcgaagacgtgctctagttactgctggtacctacatcattcaccgtcgagcacc

SEQ ID NO: 23 - DNA, 51 bp, artificial sequence
  primer
gcactcaattcgattcctactgtagccgttggtgctcgacggtgaatgatg

SEQ ID NO: 24 - DNA, 60 bp, artificial sequence
  primer
tcgacgatcctccgaaatgaaggtgcgaggctacgacgaggctgcaatatccagatttgg

SEQ ID NO: 25 - DNA, 60 bp, artificial sequence
  primer
aatgtgttggtcgagactaacggaggcgcctggcgcagaaactgcactcaattcgattcc

SEQ ID NO: 26 - DNA, 60 bp, artificial sequence
  primer
taggctgctacacccagttgtagtaggacacccagacgaactcgacgatcctccgaaatg

SEQ ID NO: 27 - DNA, 60 bp, artificial sequence
  primer
cgtaccgcttgagtcgtaagaagtgagtgttagattttcgaataatgtgttggtcgagac

SEQ ID NO: 28 - DNA, 56 bp, artificial sequence
  primer
aaagtcaggtacgagttggctcgaccgcaatgacagtgttaggctgctacacccag

SEQ ID NO: 29 - DNA, 58 bp, artificial sequence
  primer
cgtactacaacgggttgtgtattcgtcgaggtgactgtcgtaccgcttgagtcgtaag

SEQ ID NO: 30 - DNA, 60 bp, artificial sequence
  primer
tagtagaagacgtttccctgtttaagtcgaggcaatttacacaaagtcaggtacgagttg

SEQ ID NO: 31 - DNA, 57 bp, artificial sequence
  primer
gagcgcaacctctgcaagaggacggtctgagattagggatcgtactacaacgggttg

SEQ ID NO: 32 - DNA, 57 bp, artificial sequence
  primer
aggaccattattcaaacggcgcgtcaagtgtacgttgtcctagtagaagacgtttcc

SEQ ID NO: 33 - DNA, 47 bp, artificial sequence
  primer
gatcgaatcaagtgccgcgttgtagaaatgagcgcaacctctgcaag

SEQ ID NO: 34 - DNA, 37 bp, artificial sequence
  primer
gatcctcgagtgggccgaggaggaccattattcaaac

SEQ ID NO: 35 - DNA, 31 bp, artificial sequence
  primer
gatcctcgagaagtgccgcgttgtagaaatg

SEQ ID NO: 36 - DNA, 33 bp, artificial sequence
  primer
gatcgaattctgggccgaggaggaccattattc

SEQ ID NO: 37 - DNA, 59 bp, artificial sequence
  primer
gatcgaattctttttttttttttttttttttttttctgggccgaggaggaccattattc

SEQ ID NO: 38 - DNA, 70 bp, artificial sequence
  primer
tgtttgacttgcaatatagggaactttggaataggaaccaaagttgcggctcagcgctcatagagacact

SEQ ID NO: 39 - DNA, 70 bp, artificial sequence
  primer
agtgtctctatgagcgctgagccgcaactttggttcctattccaaagttccctatattgcaagtcaaaca

SEQ ID NO: 40 - DNA, 59 bp, artificial sequence
  primer
tgtgcggggctagtgtatgtctagcgacggcaaaagaaagtgtttgacttgcaatatag

SEQ ID NO: 41 - DNA, 60 bp, artificial sequence
  primer
gtgataattcgggtcaagcttattagtcgtatcaactctagtgtctctatgagcgctgag

SEQ ID NO: 42 - DNA, 59 bp, artificial sequence
  primer
cgaaagaaacttgccgcactagcgggtgtcgtagtggtattgtgcggggctagtgtatg

SEQ ID NO: 43 - DNA, 59 bp, artificial sequence
  primer
gaatgcataccctagctgagggtggactatatgatctcgtcgtgataattcgggtcaag

SEQ ID NO: 44 - DNA, 60 bp, artificial sequence
  primer
ctgagttaacggacgtgaccgaagtacacgacgacgatcgaaagaaacttgccgcactag

SEQ ID NO: 45 - DNA, 60 bp, artificial sequence
  primer
atatgagtaggggtagcggaagggttgtatgtcagatgcagaatgcataccctagctgag

SEQ ID NO: 46 - DNA, 59 bp, artificial sequence
  primer
tcaacaggtgagtccaggcctggtacgatcatcgtctcggctgagttaacggacgtgac

SEQ ID NO: 47 - DNA, 57 bp, artificial sequence
  primer
ctgagtatggctgcgaattgccctcataacacttgatatgagtaggggtagcggaag

SEQ ID NO: 48 - DNA, 52 bp, artificial sequence
  primer
tgttgattaccgtacctcttctagcttgtcaagtataatcaacaggtgagtc

SEQ ID NO: 49 - DNA, 60 bp, artificial sequence
  primer
tgcctcgacttacggtcatcaccacccaagcgggcgaaatctgagtatggctgcgaattg

SEQ ID NO: 50 - DNA, 53 bp, artificial sequence
  primer
gatcgaattcgcgttacagcctcaccccctgttgattaccgtacctcttctag

SEQ ID NO: 51 - DNA, 50 bp, artificial sequence
  primer
gatcctcgagttgagctttcacagggcacgtgcctcgacttacggtcatc

SEQ ID NO: 52 - DNA, 34 bp, artificial sequence
  primer
gatcctcgaggcgttacagcctcaccccctgttg

SEQ ID NO: 53 - DNA, 32 bp, artificial sequence
  primer
gatcgaattcttgagctttcacagggcacgtg

SEQ ID NO: 54 - DNA, 55 bp, artificial sequence
  primer
gatcgaattctttttttttttttttttttttttttcttgagctttcacagggcac

SEQ ID NO: 55 - DNA, 69 bp, artificial sequence
  primer
atcggcagttatggccatataatggttggagccaatcatttacattgtctgaggcggacgcacatctta

SEQ ID NO: 56 - DNA, 70 bp, artificial sequence
  primer
ttaagatgtgcgtccgcctcagacaatgtaaatgattggctccaaccattatatggccataactgccgat

SEQ ID NO: 57 - DNA, 59 bp, artificial sequence
  primer
tatatagtgtccagtctgaggtgtttactcgacacatcggcagttatggccatataatg

SEQ ID NO: 58 - DNA, 59 bp, artificial sequence
  primer
gaaggtacaaacactccagtccggatgtctggtcgtttcttaagatgtgcgtccgcctc

SEQ ID NO: 59 - DNA, 58 bp, artificial sequence
  primer
caaccccgcaaccaggaccccgagcccaaaatacgagtcgtatatagtgtccagtctg

SEQ ID NO: 60 - DNA, 60 bp, artificial sequence
  primer
ccatcatccgacccggggtcatgttaaaatattgaaggtacaaacactccagtccggatg

SEQ ID NO: 61 - DNA, 60 bp, artificial sequence
  primer
cttcacgtgttcagttgcgcttgactgttgatagatactcgtcaaccccgcaaccaggac

SEQ ID NO: 62 - DNA, 60 bp, artificial sequence
  primer
cgacccccatatactcgacacatcgaggtagcatccgcacccatcatccgacccggggtc

SEQ ID NO: 63 - DNA, 60 bp, artificial sequence
  primer
ggtgaatgctgaaggctgttcctagtgcgtctccacttcacgtgttcagttgcgcttgac

SEQ ID NO: 64 - DNA, 60 bp, artificial sequence
  primer
gaacgcgaccacaccgaacgaggcgcctgatgtgctcgacccccatatactcgacacatc

SEQ ID NO: 65 - DNA, 54 bp, artificial sequence
  primer
cgacatgtgcacgatatggtttcaaaagaacggggtgaatgctgaaggctgttc

SEQ ID NO: 66 - DNA, 60 bp, artificial sequence
  primer
gcgacccagaccgcacagacttgtagtccatgatataacaagaacgcgaccacaccgaac

SEQ ID NO: 67 - DNA, 58 bp, artificial sequence
  primer
gatcgaattcaaaactgtgagcacgtctcaaaatcaaactcgacatgtgcacgatatg

SEQ ID NO: 68 - DNA, 56 bp, artificial sequence
  primer
gatcctcgagcggagccatcacaagtcgtagtcacagcgacccagaccgcacagac

SEQ ID NO: 69 - DNA, 35 bp, artificial sequence
  primer
gatcctcgagaaaactgtgagcacgtctcaaaatc

SEQ ID NO: 70 - DNA, 33 bp, artificial sequence
  primer
gatcgaattccggagccatcacaagtcgtagtc

SEQ ID NO: 71 - DNA, 57 bp, artificial sequence
  primer
gatcgaattctttttttttttttttttttttttttccggagccatcacaagtcgtag

SEQ ID NO: 72 - DNA, 70 bp, artificial sequence
  primer
gctagccacactgttatgaggcggtcgagggaatcacgccaacacaaccgcacgaatggaggccgtcaaa

SEQ ID NO: 73 - DNA, 70 bp, artificial sequence
  primer
tttgacggcctccattcgtgcggttgtgttggcgtgattccctcgaccgcctcataacagtgtggctagc

SEQ ID NO: 74 - DNA, 60 bp, artificial sequence
  primer
attggtcacttactcgggtctcctgggcccctcactttctctgctagccacactgttatg

SEQ ID NO: 75 - DNA, 60 bp, artificial sequence
  primer
acaatcgccggggtgagcttacacttgcctgccttttgacggcctccattcgtgcggttg

SEQ ID NO: 76 - DNA, 60 bp, artificial sequence
  primer
aatatcagaccgccgacgactaaccagctagacaaggactattggtcacttactcgggtc

SEQ ID NO: 77 - DNA, 58 bp, artificial sequence
  primer
gagtgaagtattgaccggacctcaacgaaaagtttgtccctacaatcgccggggtgag

SEQ ID NO: 78 - DNA, 60 bp, artificial sequence
  primer
ctttggtgggtcgggaagtatatcagcactttcggggtacaatatcagaccgccgacgac

SEQ ID NO: 79 - DNA, 60 bp, artificial sequence
  primer
ggaattgctggactgtcgcccccctctatcattcatgacgagtgaagtattgacccggac

SEQ ID NO: 80 - DNA, 59 bp, artificial sequence
  primer
tacaactaggcggtacggcttttttataagacacaattctgctttggtgggtcgggaag

SEQ ID NO: 81 - DNA, 58 bp, artificial sequence
  primer
gcggtggcgcaggtgagtgcatagaatagtaaaaccctcttggaattgctggactgtc

SEQ ID NO: 82 - DNA, 48 bp, artificial sequence
  primer
catttgcccagagttcgttcaccatcagatcgtacaactaggcggtac

SEQ ID NO: 83 - DNA, 59 bp, artificial sequence
  primer
tttcccaaagatcgatttcttattcacaggcaccgatcgagcggtggcgcaggtgagtg

SEQ ID NO: 84 - DNA, 52 bp, artificial sequence
  primer
gatcgaattcaatgacggttacgagaacaacatttgcccagagttcgttcac

SEQ ID NO: 85 - DNA, 49 bp, artificial sequence
  primer
gatcctcgagtcagtgcaccatactatgaatttcccaaagatcgatttc

SEQ ID NO: 86 - DNA, 31 bp, artificial sequence
  primer
gatcctcgagaatgacggttacgagaacaac

SEQ ID NO: 87 - DNA, 34 bp, artificial sequence
  primer
gatcgaattctcagtgcaccatactatgaatttc

SEQ ID NO: 88 - DNA, 54 bp, artificial sequence
  primer
gatcgaattctttttttttttttttttttttttttctcagtgcaccatactatg

SEQ ID NO: 89 - DNA, 70 bp, artificial sequence
  primer
acccactgccaggagcgtcctcacgcctatgtgtcgagtaaccatagttttgaggcgtacgccgagcata

SEQ ID NO: 90 - DNA, 70 bp, artificial sequence
  primer
tatgctcggcgtacgcctcaaaactatggttactcgacacataggcgtgaggacgctcctggcagtgggt

SEQ ID NO: 91 - DNA, 60 bp, artificial sequence
  primer
tgactcggaccgtgatgggtcacatgcgtagtcaggtctgaacccactgccaggagcgtc

SEQ ID NO: 92 - DNA, 58 bp, artificial sequence
  primer
gctttgcattccgtcgataagcctaccaagagacaggtgtatgctcggcgtacgcctc

SEQ ID NO: 93 - DNA, 60 bp, artificial sequence
  primer
gatcactgtggtatggccctgggacgcacatgcacagttttgactggaccgtgatgggtc

SEQ ID NO: 94 - DNA, 60 bp, artificial sequence
  primer
ccaaaaggcgccagcctttgcgagctcgggccgatcagagctttgcattccgtcgataag

SEQ ID NO: 95 - DNA, 60 bp, artificial sequence
  primer
aacaaacgaagtcgtggacttgtgctgctcaattgtgttgatcactgtggtatggccctg

SEQ ID NO: 96 - DNA, 55 bp, artificial sequence
  primer
gtggtcacatcagcggactcggtttataatcccaaaaggcgccagcctttgcgag

SEQ ID NO: 97 - DNA, 58 bp, artificial sequence
  primer
agagacagtaagtcgttcgaagaatggcgctacgacaacaaacgaagtcgtggacttg

SEQ ID NO: 98 - DNA, 57 bp, artificial sequence
  primer
tacattagatgaaagcgattcattgggttgttcaagtaggtggtcacatcagcggac

SEQ ID NO: 99 - DNA, 52 bp, artificial sequence
  primer
acgagtcaaatgctctcgcaactcgcagttaattagagacagtaagtcgttc

SEQ ID NO: 100 - DNA, 58 bp, artificial sequence
  primer
cgtaatttctcttgccctaccttacaattctccgtcctacattagatgaaagcgattc

SEQ ID NO: 101 - DNA, 58 bp, artificial sequence
  primer
gatcgaattcgagatattgtacactaaaccaaatggacgagtcaaatgctctcgcaac

SEQ ID NO: 102 - DNA, 60 bp, artificial sequence
  primer
gatcctcgagtgcacgggccttacgaaccggcaataggatcgtaatttctcttgccctac

SEQ ID NO: 103 - DNA, 35 bp, artificial sequence
  primer
gatcctcgaggagatattgtacactaaaccaaatg

SEQ ID NO: 104 - DNA, 37 bp, artificial sequence
  primer
gatcgaattctgcacgggccttacgaaccggcaatag

SEQ ID NO: 105 - DNA, 54 bp, artificial sequence
  primer
gatcgaattctttttttttttttttttttttttttctgcacgggccttacgaac

SEQ ID NO: 106 - DNA, 70 bp, artificial sequence
  primer
gctttctcaaggcaatgggactgtggtggtgaaaagtttttatcttcatggggcactatcagctatcgga

SEQ ID NO: 107 - DNA, 70 bp, artificial sequence
  primer
tccgatagctgatagtgccccatgaagataaaaacttttcaccaccacagtcccattgccttgagaaagc

SEQ ID NO: 108 - DNA, 58 bp, artificial sequence
  primer
cggcagtcaacgtagttctggagcaaattaacccagctttctcaaggcaatgggactg

SEQ ID NO: 109 - DNA, 59 bp, artificial sequence
  primer
ggggattctgctctcgccactagtttatccactccgatagctgatagtgccccatgaag

SEQ ID NO: 110 - DNA, 60 bp, artificial sequence
  primer
gcaaagatggtcaaactaatggtgtacttacccaagtttacggcagtcaacgtagttctg

SEQ ID NO: 111 - DNA, 60 bp, artificial sequence
  primer
acactcctcaggtggctacctgctcggtgtcgatctgtggggggattctgctctcgccac

SEQ ID NO: 112 - DNA, 60 bp, artificial sequence
  primer
tagctatgcagggccgactccggcctcaatcgtgacacagcaaagatggtcaaactaatg

SEQ ID NO: 113 - DNA, 60 bp, artificial sequence
  primer
caatcaaaggcgccacaattattgcacatatctgaggtacactcctcaggtggctacctg

SEQ ID NO: 114 - DNA, 60 bp, artificial sequence
  primer
ctggcccttcgggtacgagcttgatggagtttgcaagtgttagctatgcagggccgactc

SEQ ID NO: 115 - DNA, 59 bp, artificial sequence
  primer
caacgcgtcacacactactagactctctatagcaacaatcaaaggcgccacaattattg

SEQ ID NO: 116 - DNA, 60 bp, artificial sequence
  primer
accaggcttgtcctcataccgcgtggaaggatgaactgtgactggcccttcgggtacgag

SEQ ID NO: 117 - DNA, 59 bp, artificial sequence
  primer
ggccgtcacaaatcagtagcaagtaagaaggtgttacacaacaacgcgtcacacactac

SEQ ID NO: 118 - DNA, 49 bp, artificial sequence
  primer
gatcctcgagtttagtcaggagtgagaagaaccaggcttgtcctcatac

SEQ ID NO: 119 - DNA, 58 bp, artificial sequence
  primer
gatcgaattcgaatctcggcgggggagtagtgggctcgcggccgtcacaaatcagtag

SEQ ID NO: 120 - DNA, 56 bp, artificial sequence
  primer
gatcgaattctttttttttttttttttttttttttcgaatctcggcgggggagtag

SEQ ID NO: 121 - DNA, 70 bp, artificial sequence
  primer
gcttgcgatataagcgtatccacgcggcacagctcgggttcgtgctgactttcgccgaccgatgtgtact

SEQ ID NO: 122 - DNA, 70 bp, artificial sequence
  primer
agtacacatcggtcggcgaaagtcagcacgaacccgagctgtgccgcgtggatacgcttatatcgcaagc

SEQ ID NO: 123 - DNA, 60 bp, artificial sequence
  primer
acattgatggcatcatgactccaatcagttagaaacagtggcttgcgatataagcgtatc

SEQ ID NO: 124 - DNA, 59 bp, artificial sequence
  primer
ttagatacgacaatgtaagggtcgtcgtgaccacaagtacacatcggtcggcgaaagtc

SEQ ID NO: 125 - DNA, 59 bp, artificial sequence
  primer
cggtggaaatttcactgttgagtgaccacatctacattgatggcatcatgactccaatc

SEQ ID NO: 126 - DNA, 58 bp, artificial sequence
  primer
agccattgaatctctgagttactgcgtctgtaacgtagtcttagatacgacctgtaag

SEQ ID NO: 127 - DNA, 60 bp, artificial sequence
  primer
gattttgggaaacactgacccaagttactagcagatcacccggtggaaatttcactgttg

SEQ ID NO: 128 - DNA, 58 bp, artificial sequence
  primer
accctgtcgttctatcggtctacgtcacttaaatggagcgagccattgaatctctgag

SEQ ID NO: 129 - DNA, 59 bp, artificial sequence
  primer
gtccctgttaactcagtgtcagtgaaacctggtagcctctgattttgggaaacactgac

SEQ ID NO: 130 - DNA, 60 bp, artificial sequence
  primer
taggagaaggtaacgctaagttgttcgatttcacaaccataccctgtcgttctatcggtc

SEQ ID NO: 131 - DNA, 60 bp, artificial sequence
  primer
cgctgctctgttccttccgtcctcaaagcctcacacgctcgtccctgttaactcagtgtc

SEQ ID NO: 132 - DNA, 60 bp, artificial sequence
  primer
gctccgaagcagacgaaattcgacgtcctcagtctatcgtaaggagaaggtaacgctaag

SEQ ID NO: 133 - DNA, 53 bp, artificial sequence
  primer
gatcgaattctccagagagacgatccgcggagcgctgctctgttccttccgtc

SEQ ID NO: 134 - DNA, 55 bp, artificial sequence
  primer
gatcctcgagtacggataaccacggcagtaagctccgaagcagacgaaattcgac

SEQ ID NO: 135 - DNA, 37 bp, artificial sequence
  primer
gatcctcgagtccagagagacgatccgcggagcgctg

SEQ ID NO: 136 - DNA, 34 bp, artificial sequence
  primer
gatgaattctacggataaccacggcagtaagctc

SEQ ID NO: 137 - DNA, 54 bp, artificial sequence
  primer
gatcgaattctttttttttttttttttttttttttctacggataaccacggcag

SEQ ID NO: 138 - DNA, 70 bp, artificial sequence
  primer
agggagccgacggctacggagtactaggtaaaggagaataatcttaagcaatgggcagtttcctctgatt

SEQ ID NO: 139 - DNA, 70 bp, artificial sequence
  primer
aatcagaggaaactgcccattgcttaagattattctcctttacctagtactccgtagccgtcggctccct

SEQ ID NO: 140 - DNA, 60 bp, artificial sequence
  primer
gcatggtcacagtctcattgctcgtcacaactaagtgggagctagggagccgacggctac

SEQ ID NO: 141 - DNA, 60 bp, artificial sequence
  primer
cgactcatgtcagttcgtggagtctgacaattaatcagaggaaactgcccattgcttaag

SEQ ID NO: 142 - DNA, 60 bp, artificial sequence
  primer
ctagattaataatactaggctcggtctcaccaccagaccagcatggtcacagtctcattg

SEQ ID NO: 143 - DNA, 60 bp, artificial sequence
  primer
ctccggcttggagtcgtacggaaccaaaatctagccgtcgtcgactcatgtcagttcgtg

SEQ ID NO: 144 - DNA, 60 bp, artificial sequence
  primer
tgtctgataacaagacgcttagctctgaccgagagggacgtgctagattaataatactag

SEQ ID NO: 145 - DNA, 58 bp, artificial sequence
  primer
ctaatggcgctgtatcctctatgatggggttcggtctgactccggcttggagtcgtac

SEQ ID NO: 146 - DNA, 60 bp, artificial sequence
  primer
cgattagctgaccaatttattcagctccaacggagtagtgtctgataacaagacgcttag

SEQ ID NO: 147 - DNA, 59 bp, artificial sequence
  primer
tcgcatttgtagagcgtcagtctcgacaagagtctaatggcgctgtatcctctatgatg

SEQ ID NO: 148 - DNA, 60 bp, artificial sequence
  primer
agaagaactgtgacccacccactcataacgactcacaacgattagctgaccaatttattc

SEQ ID NO: 149 - DNA, 60 bp, artificial sequence
  primer
cgtcgagatagtgcagaatcacgctctgaaagtgtccagatcgcatttgtagagcgtcag

SEQ ID NO: 150 - DNA, 53 bp, artificial sequence
  primer
gatcgaattcgaagtcctccaaccagaagaactgtgacccacccactcataac

SEQ ID NO: 151 - DNA, 60 bp, artificial sequence
  primer
gatcctcgagtgtatgtactcttcccgcgtcgatgcggaccgtcgagatagtgcagaatc

SEQ ID NO: 152 - DNA, 34 bp, artificial sequence
  primer
gatcctcgaggaagtcctccaaccagaagaactg

SEQ ID NO: 153 - DNA, 35 bp, artificial sequence
  primer
gatcgaattctgtatgtactcttcccgcgtcgatg

SEQ ID NO: 154 - DNA, 57 bp, artificial sequence
  primer
gatcgaattctttttttttttttttttttttttttctgtatgtactcttcccgcgtc

SEQ ID NO: 155 - DNA, 70 bp, artificial sequence
  primer
cgaaggacgctacgcagctgcgagtcttgaatgatttgtactgtaatgatcatcccacccagactcttgt

SEQ ID NO: 156 - DNA, 70 bp, artificial sequence
  primer
acaagagtctgggtgggatgatcattacagtacaaatcattcaagactcgcagctgcgtagcgtccttcg

SEQ ID NO: 157 - DNA, 60 bp, artificial sequence
  primer
cctccgaatatcgtccctcgaccggggtgaccactgcgaaggacgctacgcagctgcgag

SEQ ID NO: 158 - DNA, 58 bp, artificial sequence
  primer
aggtccaacatgatcaccgtgtgacgcatcacttcacaagagtctgggtgggatgatc

SEQ ID NO: 159 - DNA, 59 bp, artificial sequence
  primer
gccgtccccaagtctagtgaccgttaactgttttccagaccctccgaatatcgtccctc

SEQ ID NO: 160 - DNA, 60 bp, artificial sequence
  primer
atatgccgccttgcagcgagaccacagagctggcttaagaggtccaacatgatcaccgtg

SEQ ID NO: 161 - DNA, 59 bp, artificial sequence
  primer
taaatccggccaagtcgctttagcacctcatgtgagccgtgccgtccccaagtctagtg

SEQ ID NO: 162 - DNA, 60 bp, artificial sequence
  primer
ccacgtagagtgccacttaacaagagcgtgcatggccacgatatgccgccttgcagcgag

SEQ ID NO: 163 - DNA, 60 bp, artificial sequence
  primer
ggttaacagtatgtgtcacaaacgtaccagctctgcctaaatccggccaagtcgctttag

SEQ ID NO: 164 - DNA, 60 bp, artificial sequence
  primer
aattcggatctatttcggtcaggttagaggcacacccctccacgtagagtgccacttaac

SEQ ID NO: 165 - DNA, 60 bp, artificial sequence
  primer
aactcactatacatttcccgaaaccatctgccaatgttcttggttaacagtatgtgtcac

SEQ ID NO: 166 - DNA, 60 bp, artificial sequence
  primer
ggtggttacagtggccatcgtgtgaggtagagcaacactaaattcggatctatttcggtc

SEQ ID NO: 167 - DNA, 56 bp, artificial sequence
  primer
gatcctcgagtttcttaagccgtaattactttaactcactatacatttcccgaaac

SEQ ID NO: 168 - DNA, 51 bp, artificial sequence
  primer
gatcgaattcatgaaccgcgaggtcgaatgaaggtggttacagtggccatc

SEQ ID NO: 169 - DNA, 56 bp, artificial sequence
  primer
gatcgaattctttttttttttttttttttttttttcatgaaccgcgaggtcgaatg

SEQ ID NO: 170 - DNA, 70 bp, artificial sequence
  primer
ccaattcgctgtaacgtaccgagcttccaacgtttcatagtaattgaatcaagaagtcggaacgtctctt

SEQ ID NO: 171 - DNA, 70 bp, artificial sequence
  primer
aagagacgttccgacttcttgattcaattactatgaaacgttggaagctcggtacgttacagcgaattgg

SEQ ID NO: 172 - DNA, 60 bp, artificial sequence
  primer
accatcagcgtagcataccaactccttgactatactgcaatccaattcgctgtaacgtac

SEQ ID NO: 173 - DNA, 60 bp, artificial sequence
  primer
tactaccgtaaatactcgtctaatcagtgtgttcgaagagacgttccgacttcttgattc

SEQ ID NO: 174 - DNA, 60 bp, artificial sequence
  primer
gcctccgaatcaggaacatgcgtcctctaagaactttaggtgaccatcagcgtagcatac

SEQ ID NO: 175 - DNA, 60 bp, artificial sequence
  primer
gtcagtttccgccctctctagaacggttaaggagtagcagtactaccgtaaatactcgtc

SEQ ID NO: 176 - DNA, 59 bp, artificial sequence
  primer
ctatccgcccgcctgtaatttcccaatttgatacattcaaatgcctccgaatcaggaac

SEQ ID NO: 177 - DNA, 60 bp, artificial sequence
  primer
gttccagacgtcatgttacgtcgagtaccgaaagggacggtcagtttccgccctctctag

SEQ ID NO: 178 - DNA, 60 bp, artificial sequence
  primer
tagagtatccgcttactctcggatgcatagtcgagtccctatccgcccgcctgtaatttc

SEQ ID NO: 179 - DNA, 60 bp, artificial sequence
  primer
gattcagcccgtacgaggaaagcgaagatgggcaagcaggcgttccagacgtcatgttac

SEQ ID NO: 180 - DNA, 58 bp, artificial sequence
  primer
tttcaactggatcatgtcaggacggtcgggattagagtatccgcttactcttcggatg

SEQ ID NO: 181 - DNA, 59 bp, artificial sequence
  primer
gcaactctttcataacttcagacccggtacgcctaccgattcagcccgtacgaggaaag

SEQ ID NO: 182 - DNA, 50 bp, artificial sequence
  primer
gatcctcgagaggcgcagagtctgccctgttttcaactggatcatgtcag

SEQ ID NO: 183 - DNA, 52 bp, artificial sequence
  primer
gatcgaattcacggaagcaacgcggaccagagagcaactctttcataacttc

SEQ ID NO: 184 - DNA, 56 bp, artificial sequence
  primer
gatcgaattctttttttttttttttttttttttttcacggaagcaacgcggaccag

SEQ ID NO: 185 - DNA, 34 bp, artificial sequence
  primer
aagtgccgcgttgtagaaatgagcgcaacctctg

SEQ ID NO: 186 - DNA, 37 bp, artificial sequence
  primer
gcgttacagcctcaccccctgttgattaccgtacctc

SEQ ID NO: 187 - DNA, 34 bp, artificial sequence
  primer
aaaactgtgagcacgtctcaaaatcaaactcgac

SEQ ID NO: 188 - DNA, 36 bp, artificial sequence
  primer
aatgacggttacgagaacaacatttgcccagagttc

SEQ ID NO: 189 - DNA, 33 bp, artificial sequence
  primer
gagatattgtacactaaaccaaatggacgagtc

SEQ ID NO: 190 - DNA, 35 bp, artificial sequence
  primer
tttagtcaggagtgagaagaaccaggcttgtcctc

SEQ ID NO: 191 - DNA, 35 bp, artificial sequence
  primer
tccagagagacgatccgcggagcgctgctctgttc

SEQ ID NO: 192 - DNA, 38 bp, artificial sequence
  primer
gaagtcctccaaccagaagaactgtgacccccccactc

SEQ ID NO: 193 - DNA, 34 bp, artificial sequence
  primer
tttcttaagccgtaattactttaactcactatac

SEQ ID NO: 194 - DNA, 36 bp, artificial sequence
  primer
aggcgcagagtctgccctgttttcaactggatcatg

SEQ ID NO: 195 - DNA, 24 bp, artificial sequence
  primer
gcgcagaaaacaagatgagattgg

SEQ ID NO: 196 - DNA, 35 bp, artificial sequence
  primer
tgggccgaggaggaccattattcaaacggcgcgtc

SEQ ID NO: 197 - DNA, 33 bp, artificial sequence
  primer
ttgagctttcacagggcacgtgcctcgacttac

SEQ ID NO: 198 - DNA, 37 bp, artificial sequence
  primer
cggagccatcacaagtcgtagtcacagcgacccagac

SEQ ID NO: 199 - DNA, 33 bp, artificial sequence
  primer
tcagtgcaccatactatgaatttcccaaagatc

SEQ ID NO: 200 - DNA, 31 bp, artificial sequence
  primer
tgcacgggccttacgaaccggcaataggatc

SEQ ID NO: 201 - DNA, 38 bp, artificial sequence
  primer
gaatctcggcgggggagtagtgggctcgcggccgtcac

SEQ ID NO: 202 - DNA, 35 bp, artificial sequence
  primer
tacggataaccacggcagtaagctccgaagcagac

SEQ ID NO: 203 - DNA, 37 bp, artificial sequence
  primer
tgtatgtactcttcccgcgtcgatgcggaccgtcgag

SEQ ID NO: 204 - DNA, 35 bp, artificial sequence
  primer
atgaaccgcgaggtcgaatgaaggtggttacagtg

SEQ ID NO: 205 - DNA, 39 bp, artificial sequence
  primer
acggaagcaacgcggaccagagagcaactctttcataac

SEQ ID NO: 206 - DNA, 24 bp, artificial sequence
  primer
aaggtgtgcacttttattcaactg

SEQ ID NO: 207 - DNA, 70 bp, artificial sequence
  control nucleic acid sequence
ccagcagtaactagagcacgtcttcgaccaaatctggatattgcagcctcgtcgtagcctcgcaccttca

SEQ ID NO: 208 - DNA, 70 bp, artificial sequence
  control nucleic acid sequence
catatcaagtgttatgagggcaattcgcagccatactcagatttcgcccgcttgggtggtgatgaccgta

SEQ ID NO: 209 - DNA, 70 bp, artificial sequence
  control nucleic acid sequence
gcgcctcgttcggtgtggtcgcgttcttgttatatcatggactacaagtctgtgcggtctgggtcgctgt

SEQ ID NO: 210 - DNA, 70 bp, artificial sequence
  control nucleic acid sequence
cggtcgagggaatcacgccaacacaaccgcacgaatggaggccgtcaaaaggcaggcaagtgtaagctca

SEQ ID NO: 211 - DNA, 70 bp, artificial sequence
  control nucleic acid sequence
acatgcgtagtcaggtctgaacccactgccaggagcgtcctcacgcctatgtgtcgagtaaccatagttt

SEQ ID NO: 212 - DNA, 70 bp, artificial sequence
  control nucleic acid sequence
cttgtcctcataccgcgtggaaggatgaactgtgactggcccttcgggtacgagcttgatggagtttgca

SEQ ID NO: 213 - DNA, 70 bp, artificial sequence
  control nucleic acid sequence
catgactccaatcagttagaaacagtggcttgcgatataagcgtatccacgcggcacagctcgggttcgt

SEQ ID NO: 214 - DNA, 70 bp, artificial sequence
  control nucleic acid sequence
ccaatttattcagctccaacggagtagtgtctgataacaagacgcttagctctgaccgagagggacgtgc

SEQ ID NO: 215 - DNA, 70 bp, artificial sequence
  control nucleic acid sequence
aacagtatgtgtcacaaacgtaccagctctgcctaaatccggccaagtcgctttagcacctcatgtgagc

SEQ ID NO: 216 - DNA, 70 bp, artificial sequence
  control nucleic acid sequence
ccccgaatcaggaacatgcgtcctctaagaactttaggtgaccatcagcgtagcataccaactccttgac

(19) United States
(12) Patent Application Publication
(10) Pub. No.: US 2007/0065874 A1
(43) Pub. Date: Mar. 22, 2007

(54) Compositions and methods comprising control nucleic acid

(75) Inventors: Rebecca Lynn Mullinax, San Diego, CA (US); Alexey Novoradovsky, San Diego, CA (US); Joseph A. Sorge, Del Mar, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/599,936

(22) Filed: Nov. 14, 2006

Related U.S. Application Data
(63) Continuation of application No. 10/222,654, filed on Aug. 16, 2002.
(60) Provisional application No. 60/312,865, filed on Aug. 16, 2001.

(51) Int. Cl. C12Q 1/68; C12P 19/34

(52) U.S. Cl. 435/6; 435/91.2

(57) ABSTRACT

The present invention relates, in part, to control nucleic acid molecules having no significant sequence homology to any known nucleic acid, and predefined G/C-content. The present invention further relates to method of using control nucleic acid molecules to validate microarray analyses, compositions comprising control nucleic acid molecules, and kits comprising control nucleic acid molecules.

### RELATED APPLICATIONS

This application is a continuation of application Ser. No. 10/222,654 filed Aug. 16, 2002, which claims the benefit of U.S. Provisional Application No. 60/312865, filed on Aug. 16, 2001. The entire teachings of the above applications are incorporated herein by reference.

### BACKGROUND OF THE INVENTION

An increasing trend in identifying differentially expressed genes is the use of nucleic acid arrays (Schena, M., D. Shalon, R. W. Davis, and P. O. Brown. (1995) Science 270: 467-470). These arrays contain hundreds or thousands of probe genes in a single format. In these experiments, test and reference mRNA are converted into labeled cDNA in a reverse transcription or chemical reaction that incorporates fluorescent or radiolabeled nucleotides. The fluorescence-labeled test and reference labeled cDNA are then hybridized to probe genes on the arrays, unhybridized cDNA removed and hybridized cDNA detected. Differences in hybridization signals correlate with differences in abundance of those genes in the mRNA used to prepare the labeled cDNA.

The use of exogenous nucleic acid controls was first introduced in 1995 by Schena and others (Schena, ibid). In these experiments, human acetylcholine receptor mRNA (ACHR) at a 1:10,000 (w/w) dilution was combined with Arabidopsis mRNA for use as an internal control. The combined mRNA were converted to labeled cDNA, hybridized to arrays spotted with Arabidopsis genes and the human ACHR gene and the hybridization signals detected. Since then, many researchers have used exogenous DNA to validate their microarray systems. These exogenous DNA include Arabidopsis thaliana (Schena, M., D. Shalon, R. Heller, A. Chai, P. O. Brown, and R. W. Davis. (1996) Proc. Natl. Acad. Sci., USA 93:10614-10619 and Heller, R. A., M. Schena, A. Chai, D. Shalon, T. Bedilion, J. Gilmore, D. E. Woolley and R. W. Davis. (1997) Proc. Natl. Acad. Sci., USA 94:2150-2155), Escherichia coli

(www.affymetrix.com/products/gc_euka_content.html), yeast intergenic regions (Chen, J. J. W., R. Wu, P -C. Yang, J -Y Huang, Y -P Sher, M -H Han, W -C Kao, P -J Lee, T. F. Chiu, F. Chang, Y- W Chu, C -W Wu and K. Peck. (1998) Genomics 51:313-324), tobacco (Yue, H., P. S. Eastman, B. B. Wang, J. Minor, M. H. Doctolero, R. L. Nuttall, R. Stack, J. W. Becker, J. R. Montgomery, M. Vainer and R. Johnston. (2001) Nucl. Acids Res. 29:e41) and bacteriophage

(www.affymetrix.com/products/gc_euka_content.html). While these controls have been useful in evaluating microarray systems, they cannot be used to study genes derived from related species because of cross hybridization between the exogenous nucleic acid controls and their homologues. In addition, the random GC content and random nucleotide sequence of these genes affect the hybridization kinetics thereby reducing the consistency, specificity and accuracy of these hybridizations.

### SUMMARY OF THE INVENTION

The invention encompasses a method for validating a hybridization reaction comprising: (a) synthesizing a nucleic acid complement of a plurality of RNA molecules comprising mRNAs and at least one control probe nucleic acid molecule, wherein the plurality of RNA molecules are templates for the synthesizing, and wherein the synthesizing is performed in the presence of a primer capable of priming nucleic acid synthesis from the mRNAs and the control probe nucleic acid molecule; (b) hybridizing the nucleic acid synthesized in (a) to a collection of target nucleic acid molecules, wherein at least one molecule of the collection is complementary to the nucleic acid synthesized from the control probe nucleic acid; and (c) detecting the nucleic acid complement of the at least one control nucleic acid hybridized to a nucleic acid molecule of the collection.

In one embodiment, the synthesizing is further performed in the presence of an enzyme which synthesizes nucleic acid from the templates.

In another embodiment, nucleic acid not specifically hybridized to the collection is removed from the hybridization reaction. In a preferred embodiment, nucleic acid not specifically hybridized to the collection is removed from the hybridization reaction under high stringency conditions.

In another embodiment, the control probe nucleic acid is control mRNA or DNA.

In another embodiment, the synthesizing step (a) further comprises one or more dNTPs which are detectably labeled.

In another embodiment, the detectable label is a fluorescent label.

In another embodiment, the at least one molecule of the collection complementary to the nucleic acid synthesized from the control probe nucleic acid does not hybridize to the complement of an adenine-rich region in the nucleic acid synthesized from the control probe nucleic acid.

The invention further encompasses a method of making a control target nucleic acid comprising: (a) linking a control nucleic acid molecule to a nucleic acid vector to form a recombinant nucleic acid construct; (b) introducing the construct into a host cell; (c) growing the host cell under conditions which permit replication of the construct (d) isolating the construct from the host cell; and (e) synthesizing a nucleic acid complement of the construct wherein the synthesizing is performed in the presence of (i) one or more primers capable of priming nucleic acid synthesis from the construct and (ii) an enzyme which synthesizes nucleic acid from the construct.

In one embodiment, the enzyme is a DNA polymerase.

The invention further encompasses a method of making a control probe nucleic acid comprising: (a) linking a control nucleic acid molecule to a nucleic acid vector to from a recombinant nucleic acid construct; (b) introducing the construct into a host cell; (c) growing the host cell under conditions which permit replication of the construct, (d) isolating the construct from the host cell; (e) synthesizing an mRNA copy of the construct wherein the synthesizing is performed in the presence of a first enzyme which synthesizes mRNA from the construct; and (f) synthesizing a nucleic acid complement of the mRNA wherein the synthesizing is performed in the presence of (i) one or more primers capable of priming nucleic acid synthesis from the mRNA and (ii) a second enzyme which synthesizes nucleic acid from the mRNA.

In one embodiment, the nucleic acid complement is a cDNA.

In another embodiment, the nucleic acid complement is detectably labeled.

In another embodiment, the first enzyme is an RNA polymerase.

In another embodiment, the second enzyme is a reverse transcriptase.

The invention further encompasses a method of using a control target nucleic acid comprising: (a) immobilizing the control target nucleic acid on a solid support; (b) hybridizing the control target with a control probe nucleic acid; and (c) detecting the control probe nucleic acid hybridized to the control target nucleic acid.

In one embodiment, the control probe nucleic acid is detectably labeled.

In another embodiment, the solid support is a solid surface.

The invention further encompasses a method of making a control nucleic acid comprising the steps of: (a) synthesizing a nucleic acid molecule with a random sequence and having a preselected G/C-content to produce a synthetic nucleic acid molecule; (b) comparing the nucleic acid molecule with a database of nucleic acid molecules, wherein if a nucleic acid molecule contained in the database is not at least 5% identical to the synthetic nucleic acid molecule the method proceeds to step (c); (c) synthesizing a single nucleic acid complement of the synthetic nucleic acid wherein the synthesizing is performed in the presence of i) a first primer capable of priming the synthesis from the synthetic nucleic acid molecule and ii) an enzyme which synthesizes DNA from the synthetic nucleic acid; (d) synthesizing two or more nucleic acid complements of the synthetic nucleic acid wherein the synthesizing is performed in the presence of i) a second primer capable of priming synthesis from the single nucleic acid complement synthesized in step (c) or a set of such primers, and ii) an enzyme which synthesizes nucleic acid from the synthetic nucleic acid; and (e) repeating step (d) one to seven times, each time in the presence of a different second primer or set of different second primers, whereby the repeating the synthesizing generates a control nucleic acid molecule.

In one embodiment, the second primer or set of second primers comprises a 3′-terminal region of 12-30 nt that are complementary to the 3′ 12-30 nt of a strand of the single nucleic acid complement synthesized in step (c).

In another embodiment, each different second primer or set of different second primers in step (e) comprises a 3′ terminal region of 12-30 nt that are complementary to the 3′ 12-30 nucleotides of a product of the previous performance of step (d).

In another embodiment, the method further comprises the step, after step(a), of discarding all synthetic nucleic acid molecules of step (a) that comprise more than 5 contiguous G nucleotides, more than 5 contiguous C nucleotides, more than 6 contiguous A nucleotides, more than 6 contiguous T nucleotides, or more than 3 tandem repeats of any di-, tri-, or tetranucleotide sequence.

In another embodiment, step (a) further comprises the steps of: (i) generating 20 nucleotides of nucleic acid sequence, wherein the sequence has a 50% G/C content and wherein the sequence further comprises fewer than 6 contiguous G nucleotides, fewer than 6 contiguous C nucleotides, fewer than 7 contiguous A nucleotides, fewer than 7 contiguous T nucleotides, and fewer than 4 tandem repeats of any di-, tri-, or tetranucleotide sequence; (ii) cleaving the 20 nucleotide nucleic acid sequence at least two times (e.g., 2 times, 3 times, 4 times, 5 times, etc.) at random positions; and (iii) ligating the cleaved sequences to produce a ligated sequence that is different from that of the nucleic acid sequence generated in step (a), and wherein the ligated sequence comprises fewer than 6 contiguous G nucleotides, fewer than 6 contiguous C nucleotides, fewer than 7 contiguous A nucleotides, fewer than 7 contiguous T nucleotides, and fewer than 4 tandem repeats of any di-, tri-, or tetranucleotide sequence.

In another embodiment, the step of synthesizing a synthetic nucleic acid sequence further comprises the steps of i) generating a plurality of nucleic acid sequences 20 nucleotides in length wherein the sequences have a 50% G/C-content and wherein said sequences further do not include long repeats of mono, di-, tri- or tetranucleotide sequences (i.e., sequences of low complexity); ii) cleaving each of the 20 nucleotide sequences at least two, and preferably multiple times (e.g., 3, 4, 5, 6, etc.) at random positions, and iii) ligating the cleaved sequences wherein the ligated sequences do not include long repeats of mono, di-, tri- or tetranucleotide sequences (i.e., sequences of low complexity).

In another embodiment, the primer capable of priming the synthesis from the preselected nucleic acid molecule further comprises nucleotide sequences that are not complementary to the preselected nucleic acid and sequences that are not complementary to the preselected nucleic acid molecule.

In another embodiment, step (d) is a PCR reaction.

In another embodiment, the enzyme is a DNA polymerase.

The invention further encompasses a method of using a control nucleic acid comprising: (a) mixing a known amount of the control nucleic acid with one or more non-control nucleic acid molecules; and (b) detecting the control nucleic acid.

In one embodiment, the control nucleic acid is detectably labeled.

The invention further encompasses a method of using a control nucleic acid comprising: (a) mixing a known amount of the control nucleic acid with one or more isolated RNA molecules; (b) synthesizing two or more copies of the control nucleic acid and the one or more isolated RNA molecules, wherein the synthesizing is performed in the presence of i) primers capable of priming the synthesis from the control nucleic acid molecule and the one or more isolated RNA molecules and ii) an enzyme which synthesizes nucleic acid from the control nucleic acid and the one or more isolated RNA molecules; and (c) detecting the control nucleic acid.

In one embodiment, the control nucleic acid is detectably labeled.

The invention further encompasses an isolated synthetic nucleic acid molecule of at least 40 nucleotides in length, having less than 5% homology to any known nucleic acid sequence naturally found in a living organism, and having 20% to 80% G/C content, wherein the synthetic nucleic acid does not hybridize over a region of at least 30 contiguous nucleotides under high stringency conditions to any nucleic acid molecule other than its own complement, and wherein the synthetic nucleic acid comprises fewer than 6 contiguous G nucleotides, fewer than 6 contiguous C nucleotides, fewer than 7 contiguous A nucleotides, fewer than 7 contiguous T nucleotides, and fewer than 4 tandem repeats of any di-, tri-, or tetranucleotide sequence the invention also encompasses the complement of such a molecule.

In one embodiment, the synthetic nucleic acid molecule substantially lacks secondary structure.

In another embodiment, the isolated synthetic molecule further comprises a 3′ adenine-rich region of 10 to 200 nucleotides or the complement thereof.

In another embodiment, the isolated synthetic molecule further comprises a detectable marker.

In another embodiment, the detectable marker comprises a fluorescent moiety.

The invention further encompasses a vector comprising such a nucleic acid molecule, and a host cell comprising such a vector.

The invention further encompasses an isolated synthetic nucleic acid molecule of any one of SEQ ID NOs: 1-20 or a fragment thereof comprising at least 40 nucleotides, or the complement of the molecule or fragment thereof.

The invention further encompasses an isolated synthetic nucleic acid molecule comprising a sequence selected from the group consisting of: nucleotides 242-311 of SEQ ID NO: 1; nucleotides 401-470 of SEQ ID NO: 3; nucleotides 408-477 of SEQ ID NO: 5; nucleotides 237-306 of SEQ ID NO: 7; nucleotides 196-266 of SEQ ID NO: 9; nucleotides 27-96 of SEQ ID NO: 1; nucleotides 189-158 of SEQ ID NO: 13; nucleotides 64-133 of SEQ ID NO: 15; nucleotides 68-137 of SEQ ID NO: 17; nucleotides 135-204 of SEQ ID NO: 19; and the complement of any of these.

The invention further encompasses an isolated synthetic nucleic acid molecule selected from the group consisting of: nucleotides 242-311 of SEQ ID NO: 1; nucleotides 401-470 of SEQ ID NO: 3; nucleotides 408-477 of SEQ ID NO: 5; nucleotides 237-306 of SEQ ID NO: 7; nucleotides 196-266 of SEQ ID NO: 9; nucleotides 27-96 of SEQ ID NO: 11; nucleotides 189-158 of SEQ ID NO: 13; nucleotides 64-133 of SEQ ID NO: 15; nucleotides 68-137 of SEQ ID NO: 17; nucleotides 135-204 of SEQ ID NO: 19; and the complement of any of these.

In one embodiment, such isolated synthetic molecules further comprise a detectable marker. In a preferred embodiment, the detectable marker comprises a fluorescent moiety.

The invention further encompasses a vector comprising such a nucleic acid molecule and a host cell comprising such a vector.

The invention further encompasses an An isolated synthetic nucleic acid having 50% G/C content and lacking greater than 5% homology to any known naturally-occurring nucleic acid sequence, the nucleic acid selected from the group consisting of SEQ ID Nos. 21-22, 38-39, 55-56, 72-73, 89-90, 106-107, 121-122, 138-139, 155-156, and 169-170, or a fragment thereof comprising at least 40 nucleotides of such nucleic a acid.

The invention further encompasses a collection of nucleic acid molecules comprising a plurality of target nucleic acids and at least one control target nucleic acid molecule complementary to a control probe nucleic acid.

The invention further encompasses a collection of nucleic acid molecules comprising a plurality of target nucleic acids and at least one control target molecule complementary to a control probe nucleic acid comprising an adenine-rich region of 10 to 200 nucleotides, wherein the at least one control target nucleic acid molecule complementary to the control probe nucleic acid is not complementary to the adenine rich region of the control probe nucleic acid.

In one embodiment of either collection, the control probe nucleic acid is cDNA.

In another embodiment of either collection, the control probe nucleic acid is an RNA.

In another embodiment of either collection, the collection is immobilized on a solid substrate. In a preferred embodiment, the solid substrate is a solid surface.

The invention further encompasses a hybrid nucleic acid molecule comprising a control target nucleic acid molecule hybridized to a control probe nucleic acid molecule.

In one embodiment, the control target nucleic acid molecule is immobilized on a solid surface.

The invention further encompasses a kit containing: (a) a control probe RNA molecule; (b) a control target nucleic acid molecule complementary to the control probe RNA molecule; and (c) packaging materials therefor.

The invention further encompasses a kit containing: (a) control probe RNA molecule containing an adenine-rich region of 10 to 200 nucleotides; (b) a control target nucleic acid molecule complementary to the control probe RNA but lacking the adenine-rich region; and (c) packaging materials therefor.

In one embodiment of either kit, the control target nucleic acid is DNA.

In another embodiment of either kit, the kit further comprises an enzyme which synthesizes DNA from the control RNA probe.

As used herein, “control nucleic acid” refers to a nucleic acid molecule which has all of the six characteristics described below:

(1) A “control nucleic acid” is synthetic.

(2) A “control nucleic acid” has less than 5% homology to any nucleic acid sequence found in a living organism. Preferably, a “control nucleic acid” has 0% homology to any nucleic acid sequence found in a living organism. “Control nucleic acid” sequence homology with nucleic acid sequences from a living organism may be determined by, for example, a BLAST analysis against any known sequence database including, but not limited to the NCBI web site, Drosophila genome, dbest, dbsts, mouse ests, human ests, other ests, pdb, kabat, mito, alu, epd, yeast, E. coli, gss, GC web site, HGS, htgs, GC, nt, cds_human, cds_mouse, patnt, vector, est_human nr, est_mouse nr, est_nr, Hs.seq.all, Hs.seq.unique, Mm.seq.all, Mm.seq.unique, yeast.nt, ecoli.nt, sts, alu.n.

(3) A “control nucleic acid” molecule useful in the present invention will not hybridize over a region of at least 30 contiguous bases under high stringency conditions to any nucleic acid molecule other than to the complement of itself.

(4) A “control nucleic acid” refers to a nucleic acid molecule which has at least 20% G/C content and may have up to 80% G/C content. Thus, the G/C content of a control nucleic acid may be, for example, 30%, 40%, 50% and 60%.

(5) “Control nucleic acid” useful in the present invention may be DNA, RNA, cRNA, cDNA, mRNA, PNA, oligonucleotide, or polynucleotide, or combinations thereof, or a sequence which hybridizes under stringent conditions thereto, and may further be single- or double-stranded. “Control nucleic acid” molecules useful in the present invention are generally about 40 to 1000 nucleotides in length. Additional usefull lengths of control nucleic acids according to the invention are 200-800 nucleotides in length, 300-700 nucleotides in length, 400-600 nucleotides in length, and preferably about 500 nucleotides in length.

(6) A “control nucleic acid” useful in the present invention has a nucleic acid sequence which does not include long mono-, di-, tri-, or tetra-nucleotide repeats.

As used herein, the term “long repeat” means:

a) a mononucleotide repeat of more than 5 contiguous G nucleotides (e.g., GGGGGG);

b) a mononucleotide repeat of more than 5 contiguous C nucleotides (e.g., CCCCCC);

c) a mononucleotide repeat of more than 6 contiguous A nucleotides (e.g., AAAAAAA);

d) a mononucleotide repeat of more than 6 contiguous T nucleotides (e.g., TTTTTTT); or

e) more than 3 tandem repeats of a dinucleotide (e.g., CA), trinucleotide (e.g., CAT) or tetranucleotide (e.g., CATG) sequence.

Optionally, a “control nucleic acid” substantially lacks secondary structure. “Secondary structure”, as used herein refers to the formation of a hybrid between two or more nucleic acid molecules, or the formation of a hybrid within a single nucleic acid molecule of more than five contiguous base pairs. To the extent that any secondary structure exists in a “control nucleic acid”, the secondary structure is, preferably, unstable at or below a temperature that is less than (at least about 5° C. below and preferably 10° C. below) the Tₘ of the control nucleic acid. As used herein a control nucleic acid with “unstable” secondary structure, refers to a secondary structure wherein more than about 50%, preferably more than about 75%, and still more preferably more than about 90% of the base pairs that constitute the control nucleic acid are dissociated under low stringency conditions. As used herein in reference to “secondary structure”, the term “substantially lacks” means that more than about 80%, and preferably more than about 85% and still more preferably more than about 90% of the base pairs that constitute the control nucleic acid are dissociated under low stringency conditions.

The dissociation of base pairs, i.e., the presence of single stranded nucleic acid molecules instead of double-stranded, can be measured, for example by digesting the control nucleic acid with a single strand-specific endonuclease such as S1 nuclease or mung bean nuclease using conditions which are known to those of skill in the art (Ausubel, et al., supra), such that a control nucleic acid molecule in which at least 50% of the base pairs are dissociated, would result in an at least 50% decrease in the size of the control nucleic acid resolved by gel electrophoresis following endonuclease digestion.

As used herein an “RNA sample” refers to isolated sense and/or anti-sense ribonucleic acid which is obtained from an artificial (synthetic) or natural source, wherein a natural source refers to one or more cells of an organism, including but not limited to plant, animal, fungus, virus, bacterium and the like, or which is the sense or anti-sense complement of an isolated RNA molecule obtained from a natural source. For example, an “RNA sample” useful in the present invention can refer to an RNA molecule which is reverse transcribed from a cDNA molecule which is transcribed from an isolated RNA molecule obtained from a natural source. As used herein “control RNA” refers to a sense and/or anti-sense ribonucleic acid which is synthesized using a “control nucleic acid” molecule of the present invention as a template. A “control RNA” molecule useful in the present invention may be generated, for example, by inserting a “control nucleic acid” sequence into a suitable vector, known to those of skill in the art, and transcribing the “control nucleic acid” sequence so as to synthesize a “control RNA” (mRNA) molecule.

As used herein, the term “polynucleotide(s)” generally refers to any polyribonucleotide or poly-deoxyribonucleotide, which may be unmodified RNA or DNA or modified RNA or DNA. “Polynucleotide(s)” include, without limitation, single- and double-stranded nucleic acids. As used herein, the term “polynucleotide(s)” also includes DNAs or RNAs as described above that contain one or more modified bases. Thus, DNAs or RNAs with backbones modified for stability, such as peptide nucleic acid (PNA), or for other reasons are “polynucleotide(s)”. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as “oligonucleotide(s)”. A polynucleotide according to the invention may vary from 10 bases to 10 kilobases, or 100 kilobases or more in length and may be single or double stranded.

As used herein, “complementary” nucleic acid sequences are complementary to each other and can anneal by the formation of hydrogen bonds between the complementary bases.

As used herein, an “adenine rich region” refers to a stretch of nucleic acid sequence consisting of at least 10 adenine residues or a sequence complementary thereto, which is located at the 3′ terminus of a nucleic acid molecule. An “adenine rich region”, useful in the present invention is at least 10, 20, 50, 100, 150, and up to 200 residues in length. A preferred “adenine rich region” according to the present invention is a “poly-A tail” which is a stretch of at least 10 adenine residues which is appended to the 3′ end of a mRNA molecule following transcription. As used herein, an “adenine rich region” may be found in an RNA molecule, and further refers to the complementary stretch of nucleic acid residues found in a complementary DNA (cDNA) molecule.

As used herein, “detecting” as it refers to “detecting” a “control nucleic acid” hybridized to a microarray refers to a process by which the signal generated by a directly or indirectly labeled control nucleic acid is measured or observed. For example, if the detectable label is a fluorescent label, the labeled control nucleic acid is “detected” by observing or measuring the light emitted by the fluorescent label when it is excited by the appropriate wavelength, or if the detectable label is a fluorescence/quencher pair, the labeled control nucleic acid is “detected” by observing or measuring the light emitted upon dissociation of the fluorescence/quencher pair. If the detectable label is a radioactive label, the labeled control nucleic acid is “detected” by, for example, autoradiography. Methods and techniques for “detecting” fluorescent, radioactive, and other chemical labels may be found in Ausubel et al. (1995, Short Protocols in Molecular Biology, 3^{rd} Ed. John Wiley and Sons, Inc.). Alternatively, the control nucleic acid may be “indirectly detected” wherein a moiety is attached to a control nucleic acid such as an enzyme activity, allowing detection in the presence of an appropriate substrate, or a specific antigen or other marker allowing detection by addition of an antibody or other specific indicator. When hybridized to a microarray as described herein, a labeled control nucleic acid is “detected” if the measurement or observation of fluorescence or radioactive decay emitted by the detectable label is at all increased in relation to the measurement or observation of fluorescence or radioactive decay emitted when the control nucleic acid is not hybridized to the microarray.

As used herein, “high stringency conditions” refer to temperature and ionic conditions used during nucleic acid hybridization and/or washing. The extent of “high stringency” is nucleotide sequence dependent and also depends upon the various components present during hybridization. Generally, highly stringent conditions are selected to be about 5 to 20 degrees C. lower than the thermal melting point (Tₘ) for the specific sequence at a defined ionic strength and pH. Common hybridization conditions falling within the definition of “high stringency hybridization” include hybridization in 6× SSC or 6× SSPE at 68° C. in aqueous solution or at 42° C. in the presence of 50% formamide. The Tₘ is the temperature defined by the following equation: Tₘ=69.3+0.41 X (G+C)%−650/L, wherein L is the length of the probe in nucleotides. Washing is the step in which conditions are set so as to determine a minimum level of similarity between the sequences hybridizing with each other. “High stringency conditions”, as used herein, refer to a washing procedure including the incubation of two or more hybridized nucleic acids in an aqueous solution containing 0.1× SSC and 0.2% SDS, at room temperature for 2-60 minutes, followed by incubation in a solution containing 0. 1× SSC at a temperature about 12-20° C. below the calculated Tₘ of the hybrid being detected, for 2-60 minutes. “High stringency conditions” as well as factors affecting the rate of hybridization are known to those of skill in the art, and can be found in, for example, Maniatis et al., 1982, Molecular Cloning, Cold Spring Harbor Laboratory and Schena, ibid., both of which are incorporated herein by reference.

As used herein, “low stringency conditions” refer to a washing procedure including the incubation of two or more hybridized nucleic acids in an aqueous solution comprising 1× SSC and 0.2% SDS at room temperature for 2-60 minutes.

### DESCRIPTION OF THE FIGURES

FIG. 1 shows a schematic of the method used to prepare control nucleic acid molecules of the invention.

FIG. 2 shows the results of gel electrophoresis of control DNA PCR products. M: pUC19/TaqI Marker; 1-10: PCR products of control nucleic acids of SEQ ID Nos 1, 3, 5, 7, 9, 11, 13, 15, 17, or 19.

FIG. 3 shows the results of gel electrophoresis of in vitro transcribed control mRNA. M: 0.5 μg of the 0.24-9.5 KB RNA ladder (Invitrogen); 1-10: 0.5 μg of each in vitro transcribed control mRNA from the second transcription (A); 0.5 μg of in vitro transcribed control 8 mRNA from the vector that was transferred to production (B).

FIG. 4A shows a schematic diagram of template identifying the position of DNA spotted on polyL lysine-coated slides. FIG. 4B shows fluorescence-labeled control and HeLa cDNA hybridized to the corresponding control DNA that was spotted on a microarray.

FIG. 5 shows the fluorescence-labeled HeLa cDNA hybridized to an array containing either control target DNA or A. thaliana DNA.

FIG. 6A shows the template identifying the position of DNA spotted on an array: 3× SSC (B); control target DNA (P); polyA (A). FIG. 6B shows fluorescence-labeled control and HeLa cDNA hybridized to an array.

FIG. 7 shows the sequence of SEQ ID Nos: 1-20.

### DETAILED DESCRIPTION

The invention is based on the recognition that “control” nucleic acid functions as highly specific and universal hybridization control sequence in nucleic acid analysis. The lack of significant homology of the control nucleic acid to natural sequences permits the control nucleic acid to be used with any nucleic acid analysis system. The control sequences have a preselected, uniform GC content, and no long sequences of low complexity which allows for more consistent and predictable hybridization kinetics when compared to random nucleotide sequences with varying GC content. The control nucleic acid molecules can be DNA, RNA, PNA, or combinations thereof, or a nucleic acid molecule which hybridizes thereto. It is well known that DNA can form secondary structure. This secondary structure is a primary consideration in the design of control nucleic acid sequences. DNA can easily fold back upon itself to form helices and even more complicated structures. Since the concentrations of nucleic acid spotted on the arrays are high, conformations that are only slightly thermodynamically favorable can occur and influence the ability of the spotted DNA to interact with the labeled cDNA. Long runs of mono-, di-, and tri-nucleotide repeats can form secondary structures (Sugnet, C. (1999), details available at the World Wide Web site located at www.soe.ucsc.edu/˜sugnet/oligo_picker/) and are therefore avoided when the control sequences are designed. Thus, the control nucleic acid sequences of the present invention are substantially unfolded at low stringency conditions.

There is a need in the art for nucleic acid sequences which, due to their lack of significant homology to all other nucleic acid sequences, their uniform G/C content, and their lack of secondary structure, function as highly specific and universal hybridization control sequences for microarray analysis.

The present invention also provides kits comprising control nucleic acid molecules, and their complements for use in producing highly specific control hybridizations useful in microarray analysis.

Generation of Pre-Control Nucleic Acid Sequences

A control nucleic acid sequence as described herein is generated by an iterative process using randomly generated pre-control nucleic acid sequences. The randomly generated sequences were designed using a PHP4 script program running on a desktop Linux 6.2 computer, although any computer program known to those of skill in the art and capable of generating random nucleic acid sequences of a specified G/C content may be used, such as, for example, the DNAStar™ software package (DNAStar, Inc., Madison, Wis.), OLIGO 4.0 (National Biosciences, Inc.), PRIMER, Oligonucleotide Selection Program, PGEN and Amplify (described in Ausubel et al., 1995, Short Protocols in Molecular Biology, 3^{rd} Ed., John Wiley & Sons).

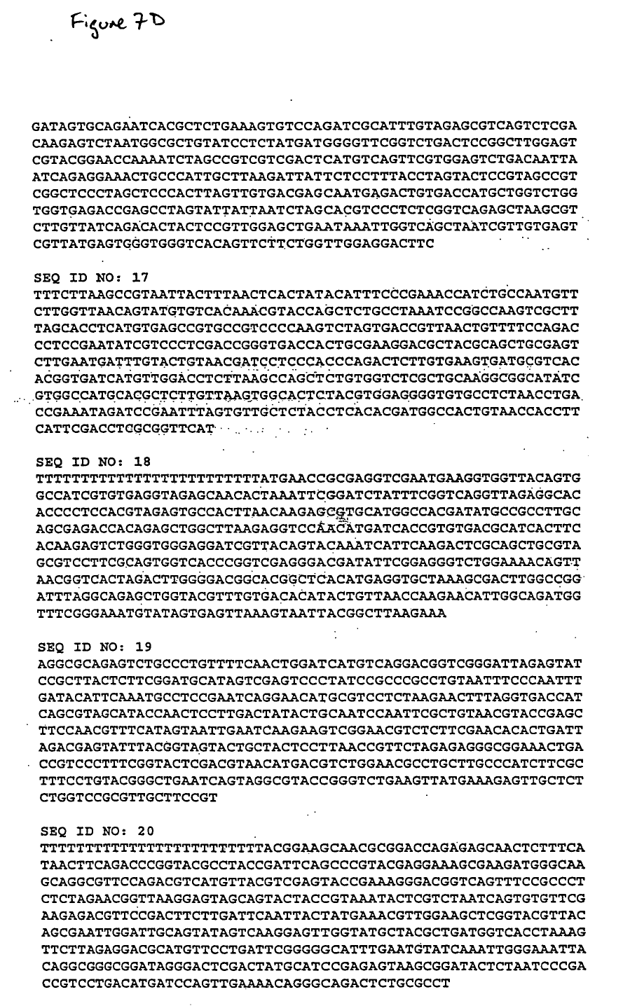

The pre-control sequences may be designed to include ten sequences for each group of different G/C-content (i.e., 20%, 25%, 30%, . . . 75%, and 80%). Ten sequences with a 50% G/C content were used to generate the control nucleic acid sequences specifically described in the present invention (SEQ ID Nos 1-20; see FIG. 7), although any of the sequences having a G/C content of between 20% and 80% may be used to generate control nucleic acid molecules according to the methods taught herein. Moreover, additional randomly generated pre-control sequences having 50% G/C content may be used to generate control nucleic acid sequences in addition to those specifically described herein used to generate control sequences 1-20 (SEQ ID Nos 1-20).

The general algorithm used to design the pre-control nucleic acid sequences described herein includes several steps. First, a “random” sequence of between 20 and 100 nucleotides is generated as described above containing a specific G/C-content. Second, the sequence is analyzed for the presence of low-complexity repeating sequence comprising mono-, di-, tri- and/or tetra-nucleotides, as it is well known to those of skill in the art that runs of bases (i.e., AAAAAAA, or GGGGGG) can form secondary structures in the nucleic acid molecule, which, as described above, is preferably avoided in the control nucleic acid sequences of the present invention. Third, the pre-control nucleic acid sequences which are accepted by the first screen, i.e., do not possess long mono-, di-, tri-, or tetra-nucleotide repeats, are optionally subjected to between about 2 and 20 cycles of random cleavage in multiple positions to generate multiple fragments of the pre-control nucleic acid sequence, followed by shuffling and recombination of the sequence fragments. Fourth, the sequence fragments are randomly re-ligated. The nucleic acid molecules may be reduced to multiple fragments by a number of different methods. The nucleic acid may be digested with an endonuclease, such as DNAse I or RNAse, or the nucleic acid molecule may be randomly sheared by sonication or passage through a syringe needle. It is also contemplated that the nucleic acid molecule may be partially or totally digested with one or more restriction enzymes, available from, for example, New England Biolabs (Beverly, Mass.), such that certain points of cross-over may be retained statistically. Methods of generating multiple nucleic acid fragments from a single nucleic acid molecule, and methods of re-ligating the fragments are known in the art and may be found, for example in U.S. Pat. No. 6,132,970 and Ausubel (supra; both of which are incorporated herein by reference in their entirety). Fifth, following ligation, the sequences are re-examined for the presence of low-complexity repeating sequence comprising mono-, di-, tri- and/or tetra-nucleotides. The sequences are subjected to the iterative process of cleavage/shuffling/ligation/screening for repeat sequence, until ten pre-control sequences are obtained which pass the screen for repeat sequences. Alternatively, instead of physically cleaving and re-ligating the sequences, the sequences may be “virtually” cleaved and re-ligated, by, for example, randomly shuffling the sequence on a computer until the pre-control sequence is obtained having the properties described above. This entire process may be repeated for each of the groups of randomly generated sequences having specified G/C-content (i.e., thereby producing ten sequences for each of the G/C-content groups which have no low-complexity repeating sequences of mono-, di-, tri-, or tetra-nucleotide repeats).

It is preferable that each of the pre-control sequences within each G/C-content group has no significant sequence similarity to each of the other sequence within the same group. In one embodiment of the present invention each sequence within a given G/C-content group has less than at least about 96% identity over greater than about 50 bases of alignable sequence with any other sequence within the same group. Preferably, each sequence within a given G/C-content group shares no more than 90%, 80%, 70%, 60%, and preferably no more than 50% identity over >50 bases of alignable sequence with any other sequence in the same group.

In one embodiment the invention relates to pre-control nucleic acid molecules having 50% G/C-content and lacking homology to any known nucleic acid sequence, and set forth in SEQ ID Nos. 21-22, 38-39, 55-56, 72-73, 89-90, 106-107, 121-122, 138-139, 155-156, and 169-170, or a fragment thereof comprising from at least about 5 nucleotides up to the full length of SEQ ID Nos. 21-22, 38-39, 55-56, 72-73, 89-90, 106-107, 121-122, 138-139, 155-156, and 169-170.

Construction of Control Nucleic Acid

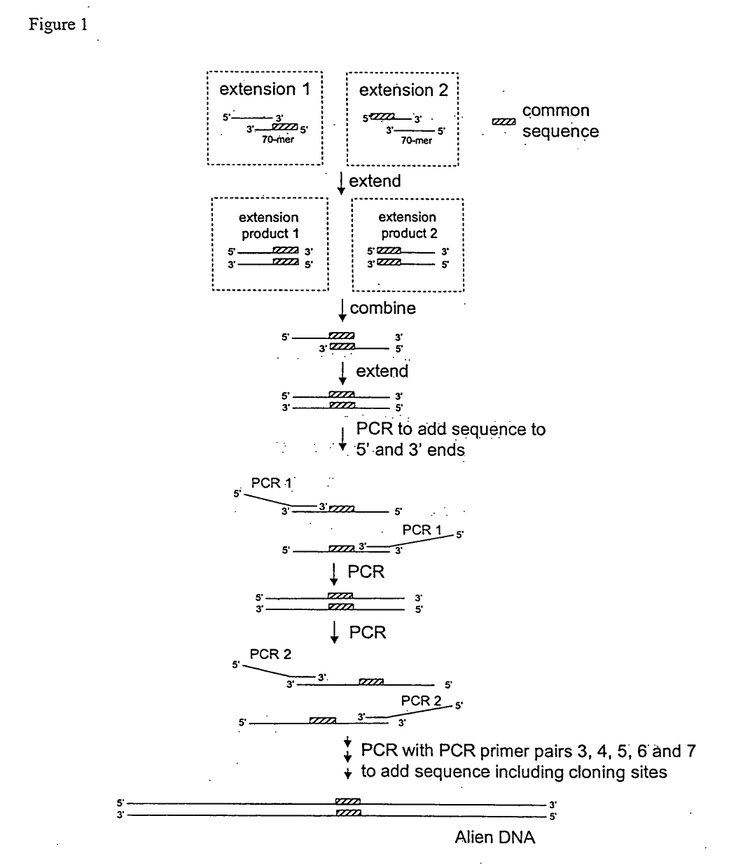

The present invention provides a method for the generation of control nucleic acid molecules using the pre-control nucleic acid molecules described above. The methods described herein may be used to generate control nucleic acid molecules using pre-control nucleic acid selected from any of the G/C-content groups described above. In general, a control nucleic acid is generated from one or more of the pre-control nucleic acid sequences by a pair of extension reactions followed by a series of amplification reactions. The overall process of generating a control nucleic acid sequence is shown schematically in FIG. 1. Briefly, each pre-control nucleic acid molecule (both the 3′-5′ and the 5′-3′ strands) selected from any of the G/C content groups described above is used in separate extension reactions along with two additional (one per extension reaction) overlapping extension oligonucleotides. The extension reaction is carried out under conditions known to those of skill in the art that are sufficient to permit the extension of the 3′ end of each of the nucleic acid molecules included in each reaction. Such conditions include, for example, a 50 μl reaction volume containing 2-3 U DNA polymerase; 200 μM each of dATP, dCTP, dGTP, and dTTP; 50-200 pmol of each pre-control nucleic acid and each overlapping extension oligonucleotide, and extension buffer such as 1× Taq PCR buffer (Stratagene, La Jolla, Calif.).

Following the first extension reaction, equimolar amounts of each of the extension products are pooled and extended a second time as shown in FIG. 1, using similar conditions to those described above. The extension reaction products may be examined by, for example, agarose gel electrophoresis to insure proper extension product size and purity. Techniques for gel electrophoresis are found in numerous laboratory texts and manuals, including, for example, Ausubel et al., supra. Alternatively, the extension reactions described above may be replaced by a PCR reaction in which the two complementary (the 3′-5′ and the 5′-3′ strands) pre-control nucleic acid molecules are amplified using the extension primers.

To generate the control nucleic acid molecules, the products of the second extension reaction may be used as a template in the first series of polymerase chain reaction amplifications. The extension reaction products are subjected to PCR using primer sets which are complementary to the 3′ end of the extension products. The product of the PCR reaction is utilized as the template in the subsequent PCR reaction, such that with each successive PCR reaction utilizing successive primer sets, the length of the PCR product is extended. PCR conditions useful for the generation of control nucleic acid molecules are known to those of skill in the art and can include for example, a 50 μl reaction volume comprising 2-3 U DNA polymerase, such as Taq, 200 μM of each dNTP, and 50-150 pmol of each oligonucleotide in 1× Taq PCR buffer (Stratagene). The specific cycling parameters used in the amplification reaction will depend on the composition, Tₘ, etc. of the primers used, but generally comprise 25-30 cycles of denaturation at 93° C. for 30 seconds, annealing at 550 C for 30 seconds, extension at 72° C. for 1 minute, followed by a final extension at 72° C. for 10 minutes to insure that all primer template hybrids are fully extended.

In one embodiment, a 17-40 nucleotide polyA tail can be added in the seventh PCR reaction. PCR conditions are similar to those described above. The polyA tail is generated by inclusion of a primer comprising a polyT segment such that when the primer is extended, a complementary polyA segment is generated. The PCR products may then be examined by, for example, agarose gel electrophoresis to insure correct size and purity, and purified using any technique known to those of skill in the art from extraction of nucleic acid from a gel, or by column purification such as the PCR High Pure Kit (Roche, Basal, Switzerland).

In one embodiment, the present invention relates to the control nucleic acid sequences of SEQ ID Nos 1-20 (see FIG. 7), or a sequence complementary thereto, generated using the pre-control nucleic acid sequences described above, and shown in Table 1 below. The control nucleic acid sequences of the present invention further encompass fragments or portions of at least 40 nucleotides up to the full length of a control nucleic acid, such as the sequences set forth in SEQ ID Nos 1-20. Exemplary useful fragments of control nucleic acid sequences of SEQ ID NOs: 1-20 are provided in Table 8 (SEQ ID NOs: 207-216).

TABLE 1

| | | | |
|---|---|---|---|
| | | | SEQ ID |
| Oligo Name | Reaction | Nucleotide Sequence (5′ to 3′) | NO |

| | | | |
|---|---|---|---|
| Control 1 | | | |
| BAS5001UC | pre-ctl. | GGTGCTCGACGGTGAATGATGTAGGTACCAGCAGTAACTAGAGCACGTCTTCGACCAAAT | 21 |
| | 1a | CTGGATATTG | |
| BAS5001LC | pre-ctl. | CAATATCCAGATTTGGTCGAAGACGTGCTCTAGTTACTGCTGGTACCTACATCATTCACC | 22 |
| | 1b | GTCGAGCACC | |
| BAS50011S | ext b | GCACTCAATTCGATTCCTACTGTAGCCGTTGGTGCTCGACGGTGAATGATG | 23 |
| BAS50011A | ext a | TCGACGATCCTCCGAAATGAAGGTGCGAGGCTACGACGAGGCTGCAATATCCAGATTTGG | 24 |
| BAS50012S | PCR 1 | AATGTGTTGGTCGAGACTAACGGAGGCGCCTGGCGCAGAAACTGCACTCAATTCGATTCC | 25 |
| BAS50012A | PCR 1 | TAGGCTGCTACACCCAGTTGTAGTAGGACACCCAGACGAACTCGACGATCCTCCGAAATG | 26 |
| BAS50013S | PCR 2 | CGTACCGCTTGAGTCGTAAGAAGTGAGTGTTAGATTTTCGAATAATGTGTTGGTCGAGAC | 27 |
| BAS50013A | PCR 2 | AAAGTCAGGTACGAGTTGGCTCGACCGCAATGACAGTGTTAGGCTGCTACACCCAG | 28 |
| BAS50014S | PCR 3 | CGTACTACAACGGGTTGTGTATTCGTCGAGGTGACTGTCGTACCGCTTGAGTCGTAAG | 29 |
| BAS50014A | PCR 3 | TAGTAGAAGACGTTTCCCTGTTTAAGTCGAGGCAATTTACACAAAGTCAGGTACGAGTTG | 30 |
| BAS50015S | PCR 4 | GAGCGCAACCTCTGCAAGAGGACGGTCTGAGATTAGGGATCGTACTACAACGGGTTG | 31 |
| BAS50015A | PCR 4 | AGGACCATTATTCAAACGGCGCGTCAAGTGTACGTTGTCCTAGTAGAAGACGTTTCC | 32 |
| BAS50016S | PCR 5 | GATCGAATCAAGTGCCGCGTTGTAGAAATGAGCGCAACCTCTGCAAG | 33 |
| BAS50016A | PCR 5 | GATCCTCGAGTGGGCCGAGGAGGACCATTATTCAAAC | 34 |
| BAS5001XI | PCR 6 & 7 | GATCCTCGAGAAGTGCCGCGTTGTAGAAATG | 35 |
| BAS5001RI | PCR 6 | GATCGAATTCTGGGCCGAGGAGGACCATTATTC | 36 |
| BAS50001A | PCR 7 | GATCGAATTCTTTTTTTTTTTTTTTTTTTTTTTTTCTGGGCCGAGGAGGACCATTATTC | 37 |
| Control 2 | | | |
| BAS5002UC | pre-ctl. | TGTTTGACTTGCAATATAGGGAACTTTGGAATAGGAACCAAAGTTGCGGCTCAGCGCTCA | 38 |
| | 2a | TAGAGACACT | |
| BAS5002LC | pre-ctl. | AGTGTCTCTATGAGCGCTGAGCCGCAACTTTGGTTCCTATTCCAAAGTTCCCTATATTGC | 39 |
| | 2b | AAGTCAAACA | |
| BAS50021S | ext b | TGTGCGGGGCTAGTGTATGTCTAGCGACGGCAAAAGAAAGTGTTTGACTTGCAATATAG | 40 |
| BAS50021A | ext a | GTGATAATTCGGGTCAAGCTTATTAGTCGTATCAACTCTAGTGTCTCTATGAGCGCTGAG | 41 |
| BAS50022S | PCR 1 | CGAAAGAAACTTGCCGCACTAGCGGGTGTCGTAGTGGTATTGTGCGGGGCTAGTGTATG | 42 |
| BAS50022A | PCR 1 | GAATGCATACCCTAGCTGAGGGTGGACTATATGATCTCGTCGTGATAATTCGGGTCAAG | 43 |
| BAS50023S | PCR 2 | CTGAGTTAACGGACGTGACCGAAGTACACGACGACGATCGAAAGAAACTTGCCGCACTAG | 44 |
| BAS50023A | PCR 2 | ATATGAGTAGGGGTAGCGGAAGGTTGTATGTCAGATGCAGAATGCATACCCTAGCTGAG | 45 |
| BAS50024S | PCR 3 | TCAACAGGTGAGTCCAGGCCTGGTACGATCATCGTCTCGGCTGAGTTAACGGACGTGAC | 46 |
| BAS50024A | PCR 3 | CTGAGTATGGCTGCGAATTGCCCTCATAACACTTGATATGAGTAGGGGTAGCGGAAG | 47 |
| BAS50025S | PCR 4 | TGTTGATTACCGTACCTCTTCTAGCTTGTCAAGTATAATCAACAGGTGAGTC | 48 |
| BAS50025A | PCR 4 | TGCCTCGACTTACGGTCATCACCACCCAAGCGGGCGAAATCTGAGTATGGCTGCGAATTG | 49 |
| BAS50026S | PCR 5 | GATCGAATTCGCGTTACAGCCTCACCCCCTGTTGATTACCGTACCTCTTCTAG | 50 |
| BAS5002SA | PCR 5 | GATCCTCGAGTTGAGCTTTCACAGGGCACGTGCCTCGACTTACGGTCATC | 51 |
| BAS5002XI | PCR 6 & 7 | GATCCTCGAGGCGTTACAGCCTCACCCCCTGTTG | 52 |
| BAS5002RI | PCR 6 | GATCGAATTCTTGAGCTTTCACAGGGCACGTG | 53 |
| BAS50002A | PCR 7 | GATCGAATTCTTTTTTTTTTTTTTTTTTTTTTTTTCTTGAGCTTTCACAGGGCAC | 54 |
| Control 3 | | | |
| BAS5003UC | pre-ctl. | ATCGGCAGTTATGGCCATATAATGGTTGGAGCCAATCATTTACATTGTCTGAGGCGGACG | 55 |
| | 3a | CACATCTTA | |
| BAS5003LC | pre-ctl. | TTAAGATGTGCGTCCGCCTCAGACAATGTAAATGATTGGCTCCAACCATTATATGGCCAT | 56 |
| | 3b | AACTGCCGAT | |
| BAS50031S | ext b | TATATAGTGTCCAGTCTGAGGTGTTTACTCGACACATCGGCAGTTATGGCCATATAATG | 57 |
| BAS50031A | ext a | GAAGGTACAAACACTCCAGTCCGGATGTCTGGTCGTTTCTTAAGATGTGCGTCCGCCTC | 58 |
| BAS50032S | PCR 1 | CAACCCCGCAACCAGGACCCCGAGCCCAAAATACGAGTCGTATATAGTGTCCAGTCTG | 59 |
| BAS50032A | PCR 1 | CCATCATCCGACCCGGGGTCATGTTAAAATATTGAAGGTACAAACACTCCAGTCCGGATG | 60 |
| BAS50033S | PCR 2 | CTTCACGTGTTCAGTTGCGCTTGACTGTTGATAGATACTCGTCAACCCCGCAACCAGGAC | 61 |
| BAS50033A | PCR 2 | CGACCCCCATATACTCGACACATCGAGGTAGCATCCGCACCCATCATCCGACCCGGGGTC | 62 |
| BAS50034S | PCR 3 | GGTGAATGCTGAAGGCTGTTCCTAGTGCGTCTCCACTTCACGTGTTCAGTTGCGCTTGAC | 63 |
| BAS50034A | PCR 3 | GAACGCGACCACACCGAACGAGGCGCCTGATGTGCTCGACCCCCATATACTCGACACATC | 64 |
| BAS50035S | PCR 4 | CGACATGTGCACGATATGGTTTCAAAAGAACGGGGTGAATGCTGAAGGCTGTTC | 65 |
| BAS50035A | PCR 4 | GCGACCCAGACCGCACAGACTTGTAGTCCATGATATAACAAGAACGCGACCACACCGAAC | 66 |
| BAS50036S | PCR 5 | GATCGAATTCAAAACTGTGAGCACGTCTCAAAATCAAACTCGACATGTGCACGATATG | 67 |
| BAS50036A | PCR 5 | GATCCTCGAGCGGAGCCATCACAAGTCGTAGTCACAGCGACCCAGACCGCACAGAC | 68 |
| BAS5003XI | PCR 6 & 7 | GATCCTCGAGAAAACTGTGAGCACGTCTCAAAATC | 69 |
| BAS5003RI | PCR 6 | GATCGAATTCCGGAGCCATCACAAGTCGTAGTC | 70 |
| BAS50003A | PCR 7 | GATCGAATTCTTTTTTTTTTTTTTTTTTTTTTTTTCCGGAGCCATCACAAGTCGTAG | 71 |
| Control 4 | | | |
| BAS5004UC | pre-ctl. | GCTAGCCACACTGTTATGAGCCGGTCGAGGGAATCACGCCAACACAACCGCACGAATGGA | 72 |
| | 4a | GGCCGTCAAA | |
| BAS5004LC | pre-ctl. | TTTGACGGCCTCCATTCGTGCGGTTGTGTTGGCGTGATTCCCTCGACCGCCTCATAACAG | 73 |
| | 4b | TGTGGCTAGC | |
| BAS50041S | ext b | ATTGGTCACTTACTCGGGTCTCCTGGGCCCCTCACTTTCTCTGCTAGCCACACTGTTATG | 74 |
| BAS50041A | ext a | ACAATCGCCGGGGTGAGCTTACACTTGCCTGCCTTTTGACGGCCTCCATTCGTGCGGTTG | 75 |
| BAS50042S | PCR 1 | AATATCAGACCGCCGACGACTAACCAGCTAGACAAGGACTATTGGTCACTTACTCGGGTC | 76 |
| BAS50042A | PCR 1 | GAGTGAAGTATTGACCGGACCTCAACGAAAAGTTTGTCCCTACAATCGCCGGGGTGAG | 77 |
| BAS50043S | PCR 2 | CTTTGGTGGGTCGGGAAGTATATCAGCACTTTCGGGGTACAATATCAGACCGCCGACGAC | 78 |
| BAS50043A | PCR 2 | GGAATTGCTGGACTGTCGCCCCCCTCTATCATTCATGACGAGTGAAGTATTGACCCGGAC | 79 |
| BAS50044S | PCR 3 | TACAACTAGGCGGTACGGCTTTTTTATAAGACACAATTCTGCTTTGGTCGGGTCGGAAG | 80 |
| BAS50044A | PCR 3 | GCGGTGGCGCAGGTGAGTGCATAGAATAGTAAAACCCTCTTGGAATTGCTGGACTGTC | 81 |
| BAS50045S | PCR 4 | CATTTGCCCAGAGTTCGTTCACCATCAGATCGTACAACTAGGCGGTAC | 82 |
| BAS50045A | PCR 4 | TTTCCCAAAGATCGATTTCTTATTCACAGGCACCGATCGAGCGGTGGCGCAGGTGAGTG | 83 |
| BAS50046S | PCR 5 | GATCGAATTCAATGACGGTTACGAGAACAACATTTGCCCAGAGTTCGTTCAC | 84 |
| BAS50046A | PCR 5 | GATCCTCGAGTCAGTGCACCATACTATGAATTTCCCAAAGATCGATTTC | 85 |
| BAS5004XI | PCR 6 & 7 | GATCCTCGAGAATGACGGTTACGAGAACAAC | 86 |
| BAS5004RI | PCR 6 | GATCGAATTCTCAGTGCACCATACTATGAATTTC | 87 |
| BAS50004A | PCR 7 | GATCGAATTCTTTTTTTTTTTTTTTTTTTTTTTTTCTCAGTGCACCATACTATG | 88 |
| Control 5 | | | |
| BAS5005UC | pre-ctl. | ACCCACTGCCAGGAGCGTCCTCACGCCTATGTGTCGAGTAACCATAGTTTTGAGGCGTAC | 89 |
| | 5a | GCCGAGCATA | |
| BAS5005LC | pre-ctl. | TATGCTCGGCGTACGCCTCAAAACTATGGTTACTCGACACATAGGCGTGAGGACGCTCCT | 90 |
| | 5b | GGCAGTGGGT | |
| BAS50051S | ext b | TGACTCGGACCGTGATGGGTCACATGCGTAGTCAGGTCTGAACCCACTGCCAGGAGCGTC | 91 |
| BAS50051A | ext a | GCTTTGCATTCCGTCGATAAGCCTACCAAGAGACAGGTGTATGCTCGGCGTACGCCTC | 92 |
| BAS50052S | PCR 1 | GATCACTGTGGTATGGCCCTGGGACGCACATGCACAGTTTTGACTGGACCGTGATGGGTC | 93 |
| BAS50052A | PCR 1 | CCAAAAGGCGCCAGCCTTTGCGAGCTCGGGCCGATCAGAGCTTTGCATTCCGTCGATAAG | 94 |
| BAS50053S | PCR 2 | AACAAACGAAGTCGTGGACTTGTGCTGCTCAATTGTGTTGATCACTGTGGTATGGCCCTG | 95 |
| BAS50053A | PCR 2 | GTGGTCACATCAGCGGACTCGGTTTATAATCCCAAAAGGCGCCAGCCTTTGCGAG | 96 |
| BAS50054S | PCR 3 | AGAGACAGTAAGTCGTTCGAAGAATGGCGCTACGACAACAAACGAAGTCGTGGACTTG | 97 |
| BAS50054A | PCR 3 | TACATTAGATGAAAGCGATTCATTGGGTTGTTCAAGTAGGTGGTCACATCAGCGGAC | 98 |
| BAS50055S | PCR 4 | ACGAGTCAAATGCTCTCGCAACTCGCAGTTAATTAGAGACGTAAGTCGTTC | 99 |
| BAS50055A | PCR 4 | CGTAATTTCTCTTGCCCTACCTTACAATTCTCCGTCCTACATTAGATGAAAGCGATTC | 100 |
| BAS50056S | PCR 5 | GATCGAATTCGAGATATTGTACACTAAACCAAATGGACGAGTCAAATGCTCTCGCAAC | 101 |
| BAS50056A | PCR 5 | GATCCTCGAGTGCACGGCCTTACGAACCGGCAATAGGATCGTAATTTCTCTTGCCCTAC | 102 |
| BAS5005XI | PCR 6 & 7 | GATCCTCGAGGAGATATTGTACACTAAACCAAATG | 103 |
| BAS5005RI | PCR 6 | GATCGAATTCTGCACGGGCCTTACGAACCGGCAATAG | 104 |
| BAS5005A | PCR 7 | GATCGAATTCTTTTTTTTTTTTTTTTTTTTTTTTTCTGCACGGGCCTTACGAAC | 105 |
| Control 6 | | | |
| BAS5006UC | pre-ctl. | GCTTTCTCAAGGCAATGGGACTGTGGTGGTGAAAAGTTTTTATCTTCATGGGGCACTATC | 106 |
| | 6a | AGCTATCGGA | |
| BAS5006LC | pre-ctl. | TCCGATAGCTGATAGTGCCCCATGAAGATAAAAACTTTTCACCACCACAGTCCCATTGCC | 107 |
| | 6b | TTGAGAAAGC | |
| BAS50061S | ext b | CGGCAGTCAACGTAGTTCTGGAGCAAATTAACCCAGCTTTCTCAAGGCAATGGGACTG | 108 |
| BAS50061A | ext a | GGGGATTCTGCTCTCGCCACTAGTTTATCCACTCCGATAGCTGATAGTGCCCCATGAAG | 109 |
| BAS50062S | PCR 1 | GCAAAGATGGTCAAACTAATGGTGTACTTACCCAAGTTTACGGCAGTCAACGTAGTTCTG | 110 |
| BAS50062A | PCR 1 | ACACTCCTCAGGTGGCTACCTGCTCGGTGTCGATCTGTGGGGGGATTCTGCTCTCGCCAC | 111 |
| BAS50063S | PCR 2 | TAGCTATGCAGGGCCGACTCCGGCCTCAATCGTGACACAGCAAAGATGGTCAAACTAATG | 112 |
| BAS50063A | PCR 2 | CAATCAAAGGCGCCACAATTATTGCACATATCTGAGGTACACTCCTCAGGTGGCTACCTG | 113 |
| BAS50064S | PCR 3 | CTGGCCCTTCGGGTACGAGCTTGATGGAGTTTGCAAGTGTTAGCTATGCAGGGCCGACTC | 114 |
| BAS50064A | PCR 3 | CAACGCGTCACACACTACTAGACTCTCTATAGCAACAATCAAAGGCGCCACAATTATTG | 115 |
| BAS50065S | PCR 4 | ACCAGGCTTGTCCTCATACCGCGTGGAAGGATGAACTGTGACTGGCCCTTCGGGTACGAG | 116 |
| BAS50065A | PCR 4 | GGCCGTCACAAATCAGTAGCAAGTAAGAAGGTGTTACACAACAACGCGTCACACACTAC | 117 |
| BAS50066S | PCR 5 & 6 | GATCCTCGAGTTTAGTCAGGAGTGAGAAGAACCACGCTTGTCCTCATAC | 118 |
| BAS50066A | PCR 5 | GATCGAATTCGAATCTCGGCGGGGGAGTAGTGGGCTCGCGGCCGTCACAAATCAGTAG | 119 |
| BAS50006A | PCR 6 | GATCGAATTCTTTTTTTTTTTTTTTTTTTTTTTTTCGAATCTCGGCGGGGGAGTAG | 120 |
| Control 7 | | | |
| BAS5007UC | pre-ctl. | GCTTGCGATATAAGCGTATCCACGCGGCACAGCTCGGGTTCGTGCTGACTTTCGCCGACC | 121 |
| | 7a | GATGTGTACT | |
| BAS5007LC | pre-ctl. | AGTACACATCGGTCGGCGAAAGTCAGCACGAACCCGAGCTGTGCCGCGTGGATACGCTTA | 122 |
| | 7b | TATCGCAAGC | |
| BAS50071S | ext b | ACATTGATGGCATCATGACTCCAATCAGTTAGAAACAGTGGCTTGCGATATAAGCGTATC | 123 |
| BAS50071A | ext a | TTAGATACGACAATGTAAGGGTCGTCGTGACCACAAGTACACATCGGTCGGCGAAAGTC | 124 |
| BAS50072S | PCR 1 | CGGTGGAAATTTCACTGTTGAGTGACCACATCTACATTGATGGCATCATGACTCCAATC | 125 |
| BAS50072A | PCR 1 | AGCCATTGAATCTCTGAGTTACTGCGTCTGTAACGTAGTCTTAGATACGACCTGTAAG | 126 |
| BAS50073S | PCR 2 | GATTTTGGGAAACACTGACCCAAGTTACTAGCAGATCACCCGGTGGAAATTTCACTGTTG | 127 |
| BAS50073A | PCR 2 | ACCCTGTCGTTCTATCGGTCTACGTCACTTAAATGGAGCGAGCCATTGAATCTCTGAG | 128 |
| BAS50074S | PCR 3 | GTCCCTGTTAACTCAGTGTCAGTGAAACCTGGTAGCCTCTGATTTTGGGAAACACTGAC | 129 |
| BAS50074A | PCR 3 | TAGGAGAAGGTAACGCTAAGTTGTTCGATTTCACAACCATACCCTGTCGTTCTATCGGTC | 130 |
| BAS50075S | PCR 4 | CGCTGCTCTGTTCCTTCCGTCCTCAAAGCCTCACACGCTCGTCCCTGTTAACTCAGTGTC | 131 |
| BAS50075A | PCR 4 | GCTCCGAAGCAGACGAAATTCGACGTCCTCAGTCTATCGTAAGGAGAAGGTAACGCTAAG | 132 |
| BAS50076S | PCR 5 | GATCGAATTCTCCAGAGAGACGATCCGCGGAGCGCTGCTCTGTTCCTTCCGTC | 133 |
| BAS50076A | PCR 5 | GATCCTCGAGTACGGATAACCACGGCAGTAAGCTCCGAAGCAGACGAAATTCGAC | 134 |
| BAS5007X1 | PCR 6 & 7 | GATCCTCGAGTCCAGAGAGACGATCCGCGGAGCGCTG | 135 |
| BAS5007RI | PCR 6 | GATGAATTCTACGGATAACCACGGCAGTAAGCTC | 136 |
| BAS50007A | PCR 7 | GATCGAATTCTTTTTTTTTTTTTTTTTTTTTTTTTCTACGGATAACCACGGCAG | 137 |
| Control 8 | | | |
| BAS5008UC | pre-ct1. | AGGGAGCCGACGGCTACGGAGTACTAGGTAAAGGAGAATAATCTTAAGCAATGGGCAGTT | 138 |
| | 8a | TCCTCTGATT | |
| BAS5008LC | pre-ctl. | AATCAGAGGAAACTGCCCATTGCTTAAGATTATTCTCCTTTACCTAGTACTCCGTAGCCG | 139 |
| | 8b | TCGGCTCCCT | |
| BAS50081S | ext b | GCATGGTCACAGTCTCATTGCTCGTCACAACTAAGTGGGAGCTAGGGAGCCGACGGCTAC | 140 |
| BAS50081A | ext a | CGACTCATGTCAGTTCGTGGAGTCTGACAATTAATCAGAGGAAACTGCCCATTGCTTAAG | 141 |
| BAS50082S | PCR 1 | CTAGATTAATAATACTAGGCTCGGTCTCACCACCAGACCAGCATGGTCACAGTCTCATTG | 142 |
| BAS50082A | PCR 1 | CTCCGGCTTGGAGTCGTACGGAACCAAAATCTAGCCGTCGTCGACTCATGTCAGTTCGTG | 143 |
| BAS50083S | PCR 2 | TGTCTGATAACAAGACGCTTAGCTCTGACCGAGAGGGACCTGCTAGATTAATAATACTAG | 144 |
| BAS50083A | PCR 2 | CTAATGGCGCTGTATCCTCTATGATGGGGTTCGGTCTGACTCCGGCTTGGAGTCGTAC | 145 |
| BAS50084S | PCR 3 | CGATTAGCTGACCAATTTATTCAGCTCCAACGGAGTAGTGTCTGATAACAAGACGCTTAG | 146 |
| BAS50084A | PCR 3 | TCGCATTTGTAGAGCGTCAGTCTCGACAAGAGTCTAATGGCGCTGTATCCTCTATGATG | 147 |
| BAS50085S | PCR 4 | AGAAGAACTGTGACCCACCCACTCATAACGACTCACAACGATTAGCTGACCAATTTATTC | 148 |
| BAS50085A | PCR 4 | CGTCGAGATAGTGCAGAATCACGCTCTGAAAGTGTCCAGATCGCATTTGTAGAGCGTCAG | 149 |
| BAS50086S | PCR 5 | GATCGAATTCGAAGTCCTCCAACCAGAAGAACTGTGACCCACCCACTCATAAC | 150 |
| BAS50086A | PCR 5 | GATCCTCGAGTGTATGTACTCTTCCCGCGTCGATGCGGACCGTCGAGATAGTGCAGAATC | 151 |
| BAS5008XI | PCR 6 & 7 | GATCCTCGAGGAAGTCCTCCAACCAGAAGAACTG | 152 |
| BAS5008RI | PCR 6 | GATCGAATTCTGTATGTACTCTTCCCGCGTCGATG | 153 |
| BAS50008A | PCR 7 | GATCGAATTCTTTTTTTTTTTTTTTTTTTTTTTTTCTGTATGTACTCTTCCCGCGTC | 154 |
| Control 9 | | | |
| BAS5009UC | pre-ctl. | CGAAGGACGCTACGCAGCTGCGAGTCTTGAATGATTTGTACTGTAATGATCATCCCACCC | 155 |
| | 9a | AGACTCTTGT | |
| BAS5009LC | pre-ctl. | ACAAGAGTCTGGGTGGGATGATCATTACAGTACAAATCATTCAAGACTCGCAGCTGCGTA | 156 |
| | 9b | GCGTCCTTCG | |
| BAS50091S | ext b | CCTCCGAATATCGTCCCTCGACCGGGGTGACCACTGCGAAGGACGCTACGCAGCTGCGAG | 157 |
| BAS50091A | ext a | AGGTCCAACATGATCACCGTGTGACGCATCACTTCACAAGAGTCTGGGTGGGATGATC | 158 |
| BAS50092S | PCR 1 | GCCGTCCCCAAGTCTAGTGACCGTTAACTGTTTTCCAGACCCTCCGAATATCGTCCCTC | 159 |
| BAS50092A | PCR 1 | ATATGCCGCCTTGCAGCGAGACCACAGAGCTGGCTTAAGAGGTCCAACATGATCACCGTG | 160 |
| BAS50093S | PCR 2 | TAAATCCGGCCAAGTCGCTTTAGCACCTCATGTGAGCCGTGCCGTCCCCAAGTCTAGTG | 161 |
| BAS50093A | PCR 2 | CCACGTAGAGTGCCACTTAACAAGAGCGTGCATGGCCACGATATGCCGCCTTGCAGCGAG | 162 |
| BAS50094S | PCR 3 | GGTTAACAGTATGTGTCACAAACGTACCAGCTCTGCCTAAATCCGGCCAAGTCGCTTTAG | 163 |
| BAS50094A | PCR 3 | AATTCGGATCTATTTCGGTCAGGTTAGAGGCACACCCCTCCACGTAGAGTGCCACTTAAC | 164 |
| BAS50095S | PCR 4 | AACTCACTATACATTTCCCGAAACCATCTGCCAATGTTCTTGGTTAACAGTATGTGTCAC | 165 |
| BAS50095A | PCR 4 | GGTGGTTACAGTGGCCATCGTGTGAGGTAGAGCAACACTAAATTCGGATCTATTTCGGTC | 166 |
| BAS50096S | PCR 5 & 6 | GATCCTCGAGTTTCTTAAGCCGTAATTACTTTAACTCACTATACATTTCCCGAAAC | 167 |
| BAS50096A | PCR 5 | GATCGAATTCATGAACCGCGAGGTCGAATGAAGGTGGTTACAGTGGCCATC | 168 |
| BAS50009A | PCR 6 | GATCGAATTCTTTTTTTTTTTTTTTTTTTTTTTTTCATGAACCGCGAGGTCGAATG | 169 |
| Control 10 | | | |
| BAS5010UC | pre-ctl. | CCAATTCGCTGTAACGTACCGAGCTTCCAACGTTTCATAGTAATTGAATCAAGAAGTCGG | 170 |
| | 10a | AACGTCTCTT | |
| BAS5010LC | pre-ctl. | AAGAGACGTTCCGACTTCTTGATTCAATTACTATGAAACGTTGGAAGCTCGGTACGTTAC | 171 |
| | 10b | AGCGAATTGG | |
| BAS50101S | ext b | ACCATCAGCGTAGCATACCAACTCCTTGACTATACTGCAATCCAATTCGCTGTAACGTAC | 172 |
| BAS50101A | ext a | TACTACCGTAAATACTCGTCTAATCAGTGTGTTCGAAGAGACGTTCCGACTTCTTGATTC | 173 |
| BAS50102S | PCR 1 | GCCTCCGAATCAGGAACATGCGTCCTCTAAGAACTTTAGGTGACCATCAGCGTAGCATAC | 174 |
| BAS50102A | PCR 1 | GTCAGTTTCCGCCCTCTCTAGAACGGTTAAGGAGTAGCAGTACTACCGTAAATACTCGTC | 175 |
| BAS50103S | PCR 2 | CTATCCGCCCGCCTGTAATTTCCCAATTTGATACATTCAAATGCCTCCGAATCAGGAAC | 176 |
| BAS50103A | PCR 2 | GTTCCAGACGTCATGTTACGTCGAGTACCGAAAGGGACGGTCAGTTTCCGCCCTCTCTAG | 177 |
| BAS50104S | PCR 3 | TAGAGTATCCGCTTACTCTCGGATGCATAGTCGAGTCCCTATCCGCCCGCCTGTAATTTC | 178 |
| BAS50104A | PCR 3 | GATTCAGCCCGTACGAGGAAAGCGAAGATGGGCAAGCAGGCGTTCCAGACGTCATGTTAC | 179 |
| BAS50105S | PCR 4 | TTTCAACTGGATCATGTCAGGACGGTCGGGATTAGAGTATCCGCTTACTCTTCGGATG | 180 |
| BAS50105A | PCR 4 | GCAACTCTTTCATAACTTCAGACCCGGTACGCCTACCGATTCAGCCCGTACGAGGAAAG | 181 |
| BAS50106S | PCR 5 & 6 | GATCCTCGAGAGGCGCAGAGTCTGCCCTGTTTTCAACTGGATCATGTCAG | 182 |
| BAS50106A | PCR 5 | GATCGAATTCACGGAAGCAACGCGGACCAGAGAGCAACTCTTTCATAACTTC | 183 |
| BAS50010A | PCR 6 | GATCGAATTCTTTTTTTTTTTTTTTTTTTTTTTTTCACGGAAGCAACGCGGACCAG | 184 |

The control nucleic acid sequence described herein may be used as positive or negative controls in, for example, microarray analysis. In one embodiment, the control nucleic acid sequences are cloned into a vector from which the control nucleic acid sequence may be amplified by PCR to generate a control DNA sequence which may be spotted onto a microarray to function as a validation control. In a further embodiment, control nucleic acid may be cloned into a second vector useful for the production of control mRNA as described above. The control mRNA may be reverse transcribed to control cDNA which may then be hybridized to the microarray comprising the control DNA. The control DNA and mRNA may be constructed as described below.

Preparation of Control PCR products

In one embodiment, the present invention provides a “control template nucleic acid” which refers to a PCR product which is generated using the control nucleic acid produced as described above as a template. In general control nucleic acid molecules may be used to generate PCR products by first inserting the control nucleic acid molecule into a suitable vector, transfecting the vector into a host cell, growing the host cell under conditions suitable for replication, isolating the control nucleic acid, and amplifying the control nucleic acid by PCR.

In one embodiment, the control nucleic acid molecules which are intended to be used to generate PCR products are constructed as described above and may or may not include an adenine-rich region or polyA tail. In a preferred embodiment, the control nucleic acid molecules which are intended to be used to generate PCR products are constructed as described above, with the exception that the primers used in the final PCR amplification do not possess a polyT region, and thus these control nucleic acid molecules do not have an adenine-rich region or a polyA tail.

Vectors

As used herein, “vector” refers to a nucleic acid molecule that is able to replicate in a host cell. A “vector” is also a “nucleic acid construct”. The terms “vector” or “nucleic acid construct” includes circular nucleic acid constructs such as plasmid constructs, cosmid vectors, etc. as well as linear nucleic acid constructs (e.g., PCR products, N15 based linear plasmids form E. coli). The nucleic acid construct may comprise expression signals such as a promoter and/or enhancer (in such a case it is referred to as an expression vector). Alternatively, a “vector” useful in the present invention can refer to an exogenous nucleic acid molecule which is integrated in the host chromosome, providing that the integrated nucleic acid molecule, in whole, or in part, can be converted back to an autonomously replicating form.

There is a wide array of vectors known and available in the art that are useful for the cloning and replication of control nucleic acid molecules according to the invention. Vectors useful according to the invention may be autonomously replicating, that is, the vector, for example, a plasmid, exists extra-chromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors.

Control nucleic acid molecules may be incorporated into one or more vectors using techniques which are well known to those of skill in the art. For example, both the control nucleic acid molecule and the appropriate vector may be digested with the either the same or compatible restriction enzymes so as to create ends on each of the molecules suitable for ligation. The insert (control nucleic acid) and vector are generally combined at an approximate 3:1 molar ratio in the presence of a DNA ligase, thus “linking” the vector and control nucleic acid molecule. Specific techniques and methods for restriction digestion and ligation are known to those of skill in the art and may be found in, for example, Maniatis et al., supra.

a. Plasmid Vectors.

Any plasmid vector that allows replication of control sequence of the invention in a selected host cell type is acceptable for use according to the invention. Plasmid vectors useful according to the invention include, but are not limited to the following examples: Bacterial—pQE70, pQE60, pQE-9 (Qiagen) pBs, phagescript, psiX174, pBluescript II SK⁺, pBluescript II KS⁺, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host. In a preferred embodiment, the vector used in the present invention for the generation of a control PCR product is pBluescript II SK⁺.

b. Bacteriophage Vectors.

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors.

A number of different viral vectors are useful according to the invention, and any viral vector that permits the introduction of one or more of the control nucleic acid sequences of the invention into cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6:616; Rosenfeld et al., 1991, Science 252:431-434; and Rosenfeld et al., 1992, Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158:97-129). An AAV vector such as that described in Traschin et al. (1985, Mol. Cell. Biol. 5:3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Host Cells

Any cell into which a recombinant vector carrying a gene encoding a control nucleic acid may be introduced and wherein the vector is permitted to replicate is useful according to the invention. Vectors suitable for the introduction of control nucleic acid sequences to host cells from a variety of different organisms, both prokaryotic and eukaryotic, are described herein above or known to those skilled in the art.

Host cells may be prokaryotic, such as any of a number of bacterial strains such as E. coli, or may be eukaryotic, such as yeast or other fungal cells, insect or amphibian cells, or mammalian cells including, for example, rodent, simian or human cells. Cells may be primary cultured cells, for example, primary human fibroblasts or keratinocytes, or may be an established cell line, such as NIH3T3, 293T or CHO cells. Further, mammalian cells useful in the present invention may be phenotypically normal or oncogenically transformed. It is assumed that one skilled in the art can readily establish and maintain a chosen host cell type in culture.

Introduction of Vectors to Host Cells.

Vectors useful in the present invention may be introduced to selected host cells by any of a number of suitable methods known to those skilled in the art. For example, vector constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1988, Current Protocols in Molecular Biology, (John Wiley & Sons, Inc., NY, N.Y.)).

For the introduction of vector constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 10⁴ colony-forming units (transformed cells)/μg of DNA. Transformed cells are then isolated on selective media appropriate to the selectable marker used.

For the introduction of vectors comprising control nucleic acid sequences to mammalian cells, the method used will depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation. These methods are detailed, for example, in Current Protocols in Molecular Biology (Ausubel et al., 1988, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, In Vitrogen, JBL Scientific, MBI Fermentas, PanVera, Promega, Quantum Biotechnologies, Sigma-Aldrich, and Wako Chemicals USA.

Following transfection, host cells useful in the present invention may be grown (i.e., cultured) under conditions known to those of skill in the art which permit replication and/or transcription of the transfected vector (see for example, Ausubel et al., supra; Maniatis et al., supra). One of skill in the art is assumed to be capable of maintaining yeast, insect, mammalian or other cells under conditions that permit vector replication and/or transcription of sequences contained therein according to the invention.

Alternatively, host cells may be screened to determine whether or not they have taken up the appropriate vector by isolating the total DNA from the cell and amplifying the DNA by PCR or equivalent method using primers specific for the vector and insert (i.e., the control nucleic acid). Methods and techniques for amplifying nucleic acid from a population of cells are well known to those of skill in the art, and may be found, for example in Innis et al., 1990, PCR Protocols: A Guide to Methods and Applications, Academic Press, Inc.

In one embodiment, host cells useful in the present invention which have been transfected with a pBluescriptII KS⁺ plasmid containing the control nucleic acid sequences of SEQ ID Nos 1-20 are screened by PCR using a 5′ insert specific primer (shown in Table 2) and a 3′ vector-specific primer (5′-TGAGCGGATAACAATTTCACACAG-3′; SEQ ID NO 205)

In addition, vectors containing the control nucleic acid insert may be distinguished from one another by restriction digestion using restriction endonucleases which are specific for the particular control nucleic acid molecule contained in the vector. However, since the sequence of some of the control nucleic acid restriction fragments is relatively small and difficult to resolve by gel electrophoresis, it is preferred that vectors containing control nucleic acid be distinguished by PCR with insert-specific primers following by confirmation by restriction digestion using techniques known in the art. In one embodiment, vectors containing the control nucleic acid having the sequence of one of SEQ ID Nos 1-20 may be distinguished from other vectors by PCR using the 5′ and 3′ insert-specific primers shown in Table 2, under appropriate amplification conditions as known to those of skill in the art, followed by restriction digestion at the unique restriction sites shown in Table 3.

TABLE 2

| | | SEQ ID | | SEQ ID |
|---|---|---|---|---|
| cDNA | 5′ PCR primer (5′ to 3′) | NO | 3′ PCR primer (5′ to 3′) | NO |
| BAS50001 | AAGTGCCGCGTTGTAGAAATGAGCGC | 185 | TGGGCCGAGGAGGACCATTATTCAAA | 196 |
| | AACCTCTG | | CGGCGCGTC | |
| BAS50002 | GCGTTACAGCCTCACCCCCTGTTGAT | 186 | TTGAGCTTTCACAGGGCACGTGCCTC | 197 |
| | TACCGTACCTC | | GACTTAC | |
| BAS50003 | AAAACTGTGAGCACGTCTCAAAATCA | 187 | CGGAGCCATCACAAGTCGTAGTCACA | 198 |
| | AACTCGAC | | GCGACCCAGAC | |
| BAS50004 | AATGACGGTTACGAGAACAACATTTG | 188 | TCAGTGCACCATACTATGAATTTCCC | 199 |
| | CCCAGAGTTC | | AAAGATC | |
| BAS50005 | GAGATATTGTACACTAAACCAAATGG | 189 | TGCACGGGCCTTACGAACCGGCAATA | 200 |
| | ACGAGTC | | GGATC | |
| BAS50006 | TTTAGTCAGGAGTGAGAAGAACCAGG | 190 | GAATCTCGGCGGGGGAGTAGTGGGCT | 201 |
| | CTTGTCCTC | | CGCGGCCGTCAC | |
| BAS50007 | TCCAGAGAGACGATCCGCGGAGCGCT | 191 | TACGGATAACCACGGCAGTAAGCTCC | 202 |
| | GCTCTGTTC | | GAAGCAGAC | |
| BAS50008 | GAAGTCCTCCAACCAGAAGAACTGTG | 192 | TGTATGTACTCTTCCCGCGTCGATGC | 203 |
| | ACCCCCCCACTC | | GGACCGTCGAG | |
| BAS50009 | TTTCTTAAGCCGTAATTACTTTAACT | 193 | ATGAACCGCGAGGTCGAATGAAGGTG | 204 |
| | CACTATAC | | GTTACAGTG | |
| HAS50010 | AGGCGCAGAGTCTGCCCTGTTTTCAA | 194 | ACGGAAGCAACGCGGACCAGAGAGCA | 205 |
| | CTGGATCATG | | ACTCTTTCATAAC | |
| X63432 | GCGCAGAAAACAAGATGAGATTGG | 195 | AAGGTGTGCACTTTTATTCAACTG | 206 |

Preparation of Control PCR Products

Once a population of host cells has been established as comprising a vector which contains a control nucleic acid sequence of the present invention, including, but not limited to the sequence of SEQ ID Nos 2, 4, 6, 8, 10, 12, 14, 16, 18, or 20, DNA is isolated from the cell population using techniques which are well established in the art including but not limited to alkaline lysis, followed by high speed centrifugation as described in Ausubel, et al., supra and Maniatis et al., supra. Alternatively, commercially available kits may be used to extract total cellular DNA from the host cells useful in the present invention including, but not limited to the MiniPrep and MaxiPrep kits available from Qiagen.

Following nucleic acid isolation, the DNA is amplified by PCR using conditions and cycling parameters similar to those described above, and which are known to those of skill in the art, or which may be found in, for example, Innis et al., 1990, PCR Protocols: A Guide to Methods and Applications, Academic Press, Inc. For example, total cellular DNA isolated from host cells comprising vectors containing the control nucleic acid sequences of SEQ ID Nos 2, 4, 6, 8, 10, 12, 14, 16, 18, 20, are amplified by PCR using control nucleic acid specific primers as shown in Table 2. Conditions for amplification of the specific control nucleic acid sequences of SEQ ID Nos 2, 4, 6, 8, 10, 12, 14, 16, 18, 20 include, but are not limited to an enzyme which synthesizes DNA from the DNA isolated from a host cell, such as 2-3 U DNA polymerase, 200 AM each dNTP, and 100 pmol of each control-specific primer shown in Table 2 in 1× TaqPlus Precision buffer (Stratagene) in a 100 μl reaction volume. Samples may be cycled according to the following parameters: denaturation at 93° C. for 30 sec.; annealing at 55° C. for 30 sec.; and extension at 72° C. for 1.5 min. for 20-30 cycles, followed by a final extension cycle at 72° C. for 10 minutes. Following amplification, the PCR products may be analyzed for appropriate size and purity by gel electrophoresis, and purified using any method known in the art, such as ethanol precipitation (Ausubel et al., supra).

Preparation of Labeled Control cDNA

As described above, one embodiment of the present invention is the use of control nucleic acid molecules as controls to validate microarray analysis, comprising spotting a control PCR product onto a microarray in addition to the control target nucleic acid spotted on the array, and hybridizing the microarray with a plurality of labeled probes wherein at least one of the probes is a “control probe nucleic acid”, which refers to a labeled cDNA synthesized from a control nucleic acid template which can hybridize to the spotted control target nucleic acid and may be used interchangably with the term “control cDNA”. The control target nucleic acid may contain a polyA-tail, but in a preferred embodiment, the control target nucleic acid does not possess an adenine-rich region or a polyA tail, thus insuring that hybridization to the control target will be specific for the control probe nucleic acid (i.e., no other probe will hybridize to the control target due to the absence of sequence homology).

Accordingly, the present invention provides a method for the generation of control mRNA and cDNA molecules, preferably labeled control mRNA or cDNA molecules which may be used to validate microarray hybridization assays. Labeled control mRNA and/or cDNA may be generated using techniques known to those of skill in the art (see, for example, Mahadevappa and Warrington, 1999, Nat. Biotech. 17: 1134; Lou et al., 1999, Nat. Med. 5:117; both of which are incorporated herein in their entirety).

Construction and Characterization of Plasmids for Preparing mRNA

In one embodiment, the present invention provides a method for cloning a control nucleic acid sequence into a vector for replication within a host cell, and the generation of mRNA molecules by in vitro transcription.

In one embodiment, the control nucleic acid molecules which are intended to be used to generate mRNA are constructed as described above and may or may not include an adenine-rich region or polyA tail. In a preferred embodiment, the control nucleic acid molecules which are intended to be used to generate mRNA are constructed as described above, with the exception that the primers used in the final PCR amplification possess a polyT region, and thus the control nucleic acid molecules have an adenine-rich region or a polyA tail.

Control nucleic acid molecules may be cloned into one or more vectors suitable for replication and/or transcription in a host cell using the methods described above for construction of a control PCR product. In addition, the control nucleic acid molecule to be used for preparation of mRNA may be cloned into the same type of vector as described above for construction of a control PCR product. In a preferred embodiment, the control nucleic acid sequences of SEQ ID Nos 1, 3, 5, 7, 9, 11, 13, 15, 17, or 19 are inserted into the vector pBluescript II KS⁺ and transformed into a suitable host cell. As described above, host cells may be screened to insure that they contain the vector comprising the control nucleic acid sequence by any method known in the art, including, but not limited to PCR using primers specific for the vector and insert (control nucleic acid). In a preferred embodiment, isolated colonies may be screened as described above with the exception that the 3′ vector-specific primer has the sequence 5′-GTTTTCCCAGTCACGACGTTG-3′ (SEQ ID NO: 206). In one embodiment, vectors containing the control nucleic acid having the sequence of one of SEQ ID Nos 1, 3, 5, 7, 9, 11, 13, 15, 17, or 19 may be distinguished from other vectors by PCR using the 5′ and 3′ insert-specific primers shown in Table 2, under appropriate amplification conditions as known to those of skill in the art, followed by restriction digestion at the unique restriction sites shown in Table 3.

TABLE 3

| | | pBluescript II SK⁺ | | pBluescript II KS⁺ | |
|---|---|---|---|---|---|
| | | PCR product plasmids | | mRNA transcript plasmids | |
| | | | Restriction | | Restriction |
| | | Restriction Site | Fragment | Restriction Site | Fragment Lengths |
| Plasmid | Enzyme | Position | Lengths (bp) | Position | (bp) |
| pBAS50001 | Kpn I | 248 | 248, 258 | | |
| pBAS50002 | Hind III | 309 | 309, 197 | | |
| pBAS50003 | Sma I | 351 | 351, 155 | | |
| pBAS50004 | Nhe I | 226 | 226, 280 | | |
| pBAS50005 | Sac I | 347 | 347, 159 | | |
| pBAS50006 | Spe I | 304 | 304, 202 | | |
| pBAS50007 | Acc I | 388 | 388, 118 | | |
| pBAS50008 | Sal I | 324 | 324, 182 | | |
| pBAS50009 | Pvu II | 240 | 240, 266 | | |
| pBAS50010 | Xba I | 349 | 349, 157 | | |
| pBAS50001A | Kpn I | | | 248 | 248, 283 |
| pBAS50002A | Hind III | | | 309 | 309, 222 |
| pBAS50003A | Sma I | | | 351 | 351, 180 |
| pBAS50004A | Nhe I | | | 226 | 226, 305 |
| pBAS50005A | Sac I | | | 347 | 347, 184 |
| pBAS50006A | Spe I | | | 304 | 304, 227 |
| pBAS50007A | Acc I | | | 388 | 388, 143 |
| pBAS50008A | ScaI | | | 324 | 324, 207 |
| pBAS50009A | Pvu II | | | 240 | 240, 291 |
| pBAS50010A | Xba I | | | 349 | 349, 182 |

Preparation of Control PolyA mRNA

Following cloning of control nucleic acid sequences into an appropriate vector, mRNA molecules may be generated by in vitro transcription, a technique which is well established in the art, and is described at least in Ausubel et al., supra. Following transcription, the quantity and quality of the control mRNA molecules may be determined by measuring the absorption at 260 and 280 nm by spectrophotometry, combined with denaturing gel electrophoresis.

Preparation of Labeled Control cDNA

As described above, one embodiment of the present invention comprises hybridizing labeled control probe nucleic acid molecules to a microarray comprising one or more control target nucleic acid molecules to serve as a validation control. Accordingly, the control mRNA generated as described above must be used to generate a labeled control cDNA molecule.

Any analytically detectable marker that is attached to or incorporated into a molecule may be used in the invention. An analytically detectable marker refers to any molecule, moiety or atom which is analytically detected and quantified.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), fluorescent/quencher pairs, radiolabels (e.g.,³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The labels may be incorporated by any of a number of means well known to those of skill in the art. However, in a preferred embodiment, the label is simultaneously incorporated during the reverse transcription of the control mRNA to generate cDNA. Thus, for example, reverse transcription using labeled primers or labeled nucleotides will provide a labeled cDNA molecule. In a preferred embodiment, transcription amplification, as described above, using a labeled nucleotide (e.g. fluorescein-labeled UTP and/or CTP) incorporates a label into the transcribed polynucleotides. In a further preferred embodiment, detectably labeled control cDNA molecules may be generated using a commercially available kit such as the FairPlay™ labeling kit (Stratagene, cat. no. 252002)

Alternatively, a label may be added directly to the control cDNA sample after the reverse transcription is completed. Means of attaching labels to polynucleotides are well known to those of skill in the art and include, for example nick translation or end-labeling (e.g. with a labeled RNA) by kinasing of the polynucleotide and subsequent attachment (ligation) of a polynucleotide linker joining the sample polynucleotide to a label (e.g., a fluorophore).

Alternatively, a label may be added directly to the control RNA sample by coupling the RNA directly to a detectable molecule. Means of attaching labels to polynucleotides are well known to those of skill in the art and include, for example incubating the RNA with a dye coujugated cis-platinum molecule.

In a preferred embodiment, the fluorescent modifications are by cyanine dyes e.g. Cy-3/Cy-5 dUTP, Cy-3/Cy-5 dCTP (Amersham Pharmacia) or alexa dyes (Khan, J., Simon, R., Bittner, M., Chen, Y., Leighton, S. B., Pohida, T., Smith, P. D., Jiang, Y., Gooden, G. C., Trent, J. M. & Meltzer, P. S. (1998) Cancer Res. 58, 50095013.).

In one embodiment, the control cDNA may be used as a template to synthesize a complementary RNA molecule (cRNA) using an enzyme such as SP6, T7 or T3 RNA polymnerase. Methods for cRNA synthesis are well known to those of skill in the art.

Preparation of Control DNA Microarrays

In one embodiment, the present invention provides a collection of nucleic acid target molecules wherein at least one of the targets is capable of hybridizing to a control cDNA molecule, preferably constructed as described above. In a preferred embodiment, the target which is capable of hybridizing to a control cDNA molecule is a control DNA molecule. In a further preferred embodiment, the collection of nucleic acid target molecules are stably associated with a solid surface such as a microarray. Any combination of the PCR products generated from control nucleic acid sequences are used for the construction of a microarray. A microarray according to the invention preferably comprises between 10 and 100,000 nucleic acid members, and more preferably comprises at least 1000 nucleic acid members. The nucleic acid members are known or novel polynucleotide sequences described herein, or any combination thereof, and including at least one nucleic acid molecule, capable of hybridizing to a control cDNA. While it is known to those of skill in the art that the nomenclature of microarray analysis describes the nucleic acid molecule stably associated with the microarray the “probe” and the nucleic acid molecule in solution hybridized thereto the “target”, the present invention is not limited only to the use of control nucleic acid sequences in microarray analysis, and thus, for purposes of the present disclosure, the control nucleic acid molecule stably associated with the microarray surface will be termed the “target” and the control nucleic acid molecule in solution hybridized thereto will be termed the “probe”; the terms “probe” and “target” for purposes of the invention are essentially interchangable.

The target nucleic acid samples that are hybridized to and analyzed with a microarray of the invention may be derived from any source known to those of skill in the art, and can include synthetic nucleic acids, provided that at least one target nucleic acid sample is capable of hybridizing with a control cDNA, and is preferably a control DNA constructed as described above.

Construction of a Microarray

In the subject methods, an array of nucleic acid members stably associated with the surface of a solid support is contacted with a sample comprising target polynucleotides under hybridization conditions sufficient to produce a hybridization pattern of complementary nucleic acid members/target complexes.

The nucleic acid members may be produced using established techniques such as polymerase chain reaction (PCR) and reverse transcription (RT). These methods are similar to those currently known in the art (see e.g. PCR Strategies, Michael A. Innis (Editor), et al. (1995) and PCR: Introduction to Biotechniques Series, C. R. Newton, A. Graham (1997)). Amplified polynucleotides are purified by methods well known in the art (e.g., column purification or alcohol precipitation). A polynucleotide is considered pure when it has been isolated so as to be substantially free of primers and incomplete products produced during the synthesis of the desired polynucleotide. Preferably, a polynucleotide will also be substantially free of contaminants which may hinder or otherwise mask the binding activity of the molecule.

In one embodiment, a control DNA molecule may be spotted onto a microarray comprising a plurality of non-control polynucleotides. In one embodiment, the non-control polynucleotides are provided by the user of the micorarray and may be spotted onto the microarray along with the control DNA of the invention. A microarray according to the invention comprises a plurality of unique polynucleotides attached to one surface of a solid support at a density exceeding 10 different polynucleotides/cm², wherein each of the polynucleotides is attached to the surface of the solid support in a non-identical preselected region. Each associated sample on the array comprises a polynucleotide composition of known identity, usually of known sequence, as described in greater detail below. Any conceivable substrate may be employed in the invention. In one embodiment, the polynucleotide attached to the surface of the solid support is DNA. In a preferred embodiment, the polynucleotide attached to the surface of the solid support is cDNA, RNA, PNA, or a combination thereof. In a preferred embodiment, the polynucleotide attached to the surface of the solid support is genomic DNA synthesized by polymerase chain reaction(PCR). In another preferred embodiment, the polynucleotide attached to the surface of the solid support is cDNA synthesized by PCR. Preferably, a nucleic acid member comprising an array, according to the invention, is at least 30 nucleotides in length. In one embodiment, a nucleic acid member comprising an array is at least 50, 70, 100, or 150 nucleotides in length. Preferably, a nucleic acid member comprising an array is less than 1000 nucleotides in length. More preferably, a nucleic acid member comprising an array is less than 500 nucleotides in length. In one embodiment, an array comprises at least 10 different polynucleotides attached to one surface of the solid support. In another embodiment, the array comprises at least 100 different polynucleotides attached to one surface of the solid support. In yet another embodiment, the array comprises at least 10,000, and up to 100,000 different polynucleotides attached to one surface of the solid support.

In the arrays of the invention, the polynucleotide compositions are stably associated with the surface of a solid support, wherein the support may be a flexible or rigid solid support. By “stably associated” is meant that each nucleic acid member maintains a unique position relative to the solid support under hybridization and washing conditions. As such, the samples are non-covalently or covalently stably associated with the support surface. Examples of non-covalent association include non-specific adsorption, binding based on electrostatic interactions (e.g., ion pair interactions), hydrophobic interactions, hydrogen bonding interactions, specific binding through a specific binding pair member covalently attached to the support surface, and the like. Examples of covalent binding include covalent bonds formed between the polynucleotides and a functional group present on the surface of the rigid support (e.g., —OH), where the functional group may be naturally occurring or present as a member of an introduced linking group, as described in greater detail below The amount of polynucleotide present in each composition will be sufficient to provide for adequate hybridization and detection of target polynucleotide sequences during the assay in which the array is employed. Generally, the amount of each nucleic acid member stably associated with the solid support of the array is at least about 0.001 ng, preferably at least about 0.01 ng and more preferably at least about 0.05 ng, where the amount may be as high as 0.1 μg or higher, but will usually not exceed about 0.1 μg. Where the nucleic acid member is “spotted” onto the solid support in a spot comprising an overall circular dimension, the diameter of the “spot” will generally range from about 10 to 5,000 μm, usually from about 20 to 2,000 μm and more usually from about 50 to 500 μm.

Control nucleic acid members in addition to the control DNA may be present on the array including nucleic acid members comprising oligonucleotides or polynucleotides corresponding to genomic DNA, housekeeping genes, vector sequence, plant nucleic acid sequence, negative and positive control genes, and the like. Control nucleic acid members, including the control DNA members are calibrating or control genes whose function is not to tell whether a particular “key” gene of interest is expressed, but rather to provide other useful information, such as background, hybridization specificity, or basal level of expression. In one embodiment, control nucleic acid members other than the control DNA of the invention are selected from the group including, but not limited to human Cot-1 DNA, salmon sperm DNA, Arabadopsis thaliana DNA, and polyA DNA.

Solid Substrate

An array according to the invention comprises either a flexible or rigid substrate. A flexible substrate is capable of being bent, folded or similarly manipulated without breakage. Examples of solid materials which are flexible solid supports with respect to the present invention include membranes, e.g., nylon, flexible plastic films, and the like. By “rigid” is meant that the support is solid and does not readily bend, i.e., the support is not flexible. As such, the rigid substrates of the subject arrays are sufficient to provide physical support and structure to the associated polynucleotides present thereon under the assay conditions in which the array is employed, particularly under high throughput handling conditions.

The substrate may be biological, non-biological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat or planar but may take on a variety of alternative surface configurations. The substrate may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in the art upon review of this disclosure.

In a preferred embodiment the substrate is flat glass or single-crystal silicon. According to some embodiments, the surface of the substrate is etched using well known techniques to provide for desired surface features. For example, by way of the formation of trenches, v-grooves, mesa structures, or the like, the synthesis regions may be more closely placed within the focus point of impinging light, be provided with reflective “mirror” structures for maximization of light collection from fluorescent sources, etc.

Surfaces on the solid substrate will usually, though not always, be composed of the same material as the substrate. Alternatively, the surface may be composed of any of a wide variety of materials, for example, polymers, plastics, resins, polysaccharides, silica or silica-based materials, carbon, metals, inorganic glasses, membranes, or any of the above-listed substrate materials. In some embodiments the surface may provide for the use of caged binding members which are attached firmly to the surface of the substrate. Preferably, the surface will contain reactive groups, which are carboxyl, amino, hydroxyl, or the like. Most preferably, the surface will be optically transparent and will have surface Si—OH functionalities, such as are found on silica surfaces.

The surface of the substrate is preferably provided with a layer of linker molecules, although it will be understood that the linker molecules are not required elements of the invention. The linker molecules are preferably of sufficient length to permit polynucleotides of the invention and on a substrate to hybridize to other polynucleotide molecules and to interact freely with molecules exposed to the substrate.

Often, the substrate is a silicon or glass surface, (poly)tetrafluoroethylene, (poly)vinylidenedifluoride, polystyrene, polycarbonate, a charged membrane, such as nylon 66 or nitrocellulose, or combinations thereof. In a preferred embodiment, the solid support is glass. Preferably, at least one surface of the substrate will be substantially flat. Preferably, the surface of the solid support will contain reactive groups, including, but not limited to, carboxyl, amino, hydroxyl, thiol, or the like. In one embodiment, the surface is optically transparent. In a preferred embodiment, the substrate is a poly-lysine coated slide or Gamma amino propyl silane-coated Corning Microarray Technology-GAPS.

Any solid support to which a nucleic acid member may be attached may be used in the invention. Examples of suitable solid support materials include, but are not limited to, silicates such as glass and silica gel, cellulose and nitrocellulose papers, nylon, polystyrene, polymethacrylate, latex, rubber, and fluorocarbon resins such as TEFLON™.

The solid support material may be used in a wide variety of shapes including, but not limited to slides and beads. Slides provide several functional advantages and thus are a preferred form of solid support. Due to their flat surface, probe and hybridization reagents are minimized using glass slides. Slides also enable the targeted application of reagents, are easy to keep at a constant temperature, are easy to wash and facilitate the direct visualization of RNA and/or DNA immobilized on the solid support. Removal of RNA and/or DNA immobilized on the solid support is also facilitated using slides.

In a preferred embodiment, the solid substrate is selected from the group consisting of, but not limited to, poly-L-lysine coated glass slides, CMT-GAPII slides (Corning), SuperAmine slides (Telechem) and dendrimer treated slides (Stratagene).

The particular material selected as the solid support is not essential to the invention, as long as it provides the described function. Normally, those who make or use the invention will select the best commercially available material based upon the economics of cost and availability, the expected application requirements of the final product, and the demands of the overall manufacturing process.

Spotting Method

The invention provides for arrays wherein each nucleic acid member comprising the array is spotted onto a solid support.

Preferably, spotting is carried out as follows. DNA molecules or PCR products (˜40 ul), including control DNA are precipitated with 4 ul ( 1/10 volume) of 3M sodium acetate (pH 5.2) and 100 ul (2.5 volumes) of ethanol and stored overnight at −20° C. They are then centrifuged at 12,000×g at 4° C. for 1 hour. The obtained pellets are washed with 50 ul ice-cold 70% ethanol and centrifuged again for 30 minutes. The pellets are then air-dried and resuspended well in 20 μl 3× SSC and incubated overnight. The samples are then spotted, either singly or in duplicate, onto polylysine-coated slides (Sigma Cat. No. P0425) using a robotic GMS 417 arrayer (Affymetrix, Calif.). In one embodiment, the spotting buffer is selected from the group including, but not limited to 3× SSC, 50% DMSO, 5% sodium bicarbonate, and 50% DMSO in 0.1× TE.

The boundaries of the spots on the microarray may be marked with a diamond scriber (note that the spots become invisible after post-processing). The arrays are rehydrated by suspending the slides over a dish of warm particle free ddH20 for approximately one minute (the spots will swell slightly but will not run into each other) and snap-dried on a 70-80° C. inverted heating block for 3 seconds. Nucleic acid is then UV crosslinked to the slide (Stratagene, Stratalinker, 65 mJ—set display to “650” which is 650×100 uJ). The arrays are placed in a slide rack. An empty slide chamber is prepared and filled with the following solution: 3.0 grams of succinic anhydride (Aldrich) was dissolved in 189 ml of 1-methyl-2-pyrrolidinone (rapid addition of reagent is crucial); immediately after the last flake of succinic anhydride is dissolved, 21.0 ml of 0.2 M sodium borate is mixed in and the solution is poured into the slide chamber. The slide rack is plunged rapidly and evenly in the slide chamber and vigorously shaken up and down for a few seconds, making sure the slides never leave the solution, and then mixed on an orbital shaker for 15-20 minutes. The slide rack is then gently plunged in 95° C. ddH20 for 2 minutes, followed by plunging five times in 95% ethanol. The slides are then air dried by allowing excess ethanol to drip onto paper towels, followed by centriftigation at 12,000×g for 5 minutes. The arrays are then stored in the slide box at room temperature until use.

Numerous methods may be used for attachment of the nucleic acid members of the invention to the substrate (a process referred as spotting). For example, polynucleotides are attached using the techniques of, for example U.S. Pat. No. 5,807,522, which is incorporated herein by reference for teaching methods of polymer attachment.

Alternatively, spotting may be carried out using contact printing technology. In one embodiment, the nucleic acid members are spotted onto the surface using a Gene Machines arrayer.

Printing Scheme

In a preferred embodiment, a pattern for printing the microarray may be devised such that the control spots (i.e., control PCR products) are present in all regions of the surface and in sufficient replicate numbers (at least greater than about 2) to permit statistical analysis. Spots of probe sequences expected to give significant hybridization signals, such as the control PCR products, may be placed in a pattern at the perimeter of the array to serve as landmarks so that it is immediately clear when looking at the array that the entire array is present and that is has been in contact with the hybridization solution. Placing positive and/or negative control spots in the four corners of the surface can also serve to provide points of reference when determining the orientation of the microarray.

Microarray Hybridization

Polynucleotide hybridization involves providing a probe nucleic acid member (i.e., control cDNA) and target polynucleotide (i.e., control PCR product) under conditions where the probe nucleic acid member and its complementary target can form stable hybrid duplexes through complementary base pairing. The polynucleotides that do not form hybrid duplexes are then washed away leaving the hybridized polynucleotides to be detected, typically through detection of an attached detectable label. It is generally recognized that polynucleotides are denatured by increasing the temperature or decreasing the salt concentration of the buffer containing the polynucleotides. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

The invention provides for hybridization conditions comprising formamide-based hybridization solutions, for example as described in Ausubel et al., supra and Sambrook et al. supra, or Hegde et al. (2000, Biotechniques, 29:548; incorporated herein by reference in its entirety), in a preferred embodiment, methods provided in the Microarray Labeling Kit (Stratagene).

Methods of optimizing hybridization conditions are well known to those of skill in the art (see, e.g., Laboratory Techniques in Biochemistry and Molecular Biology, Vol. 24: Hybridization With Polynucleotide Probes, P. Tijssen, ed. Elsevier, N.Y., (1993)).

Following hybridization, non-hybridized labeled or unlabeled polynucleotide is removed from the support surface, conveniently by washing, thereby generating a pattern of hybridized probe polynucleotide on the substrate surface. A variety of wash solutions are known to those of skill in the art and may be used. The resultant hybridization patterns of labeled, hybridized oligonucleotides and/or polynucleotides may be visualized or detected in a variety of ways, with the particular manner of detection being chosen based on the particular label of the probe polynucleotide, where representative detection means include scintillation counting, autoradiography, fluorescence measurement, calorimetric measurement, light emission measurement and the like.

Image Acquisition and Data Analysis

Following hybridization and any washing step(s) and/or subsequent treatments, as described above, the resultant hybridization pattern is detected. In detecting or visualizing the hybridization pattern, the intensity or signal value of the label will be detected and quantified, by which is meant that the signal from each spot of the hybridization will be measured.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e., data deviating from a predetermined statistical distribution, and calculating the relative abundance of the test polynucleotides from the remaining data. The resulting data is displayed as an image with the intensity in each region varying according to the abundance of the labeled control target nucleic acid.

In a preferred embodiment, fluorescence intensities of immobilized target nucleic acid sequences are determined from images taken with a custom confocal microscope equipped with laser excitation sources and interference filters appropriate for the Cy3 and Cy5 fluors. Separate scans were taken for each fluor at a resolution of 225 μm² per pixel and 65,536 gray levels. Image segmentation to identify areas of hybridization, normalization of the intensities between the two fluor images, and calculation of the normalized mean fluorescent values at each target are as described (Khan, et al., 1998, Cancer Res. 58:5009-5013. Chen, et al., 1997, Biomed. Optics 2:364-374). Normalization between the images is used to adjust for the different efficiencies in labeling and detection with the two different fluors. This is achieved by equilibrating to a value of one the signal intensity ratio of a set of one or more control nucleic acid molecules (control probe PCR products) spotted on the array.

Following detection or visualization, the hybridization pattern is used to determine quantitative information about the genetic profile of the labeled target polynucleotide sample that was contacted with the array to generate the hybridization pattern, as well as the physiological source from which the labeled target polynucleotide sample was derived. By “genetic profile” is meant information regarding the types of polynucleotides present in the sample, e.g., such as the types of genes to which they are complementary, and/or the copy number of each particular polynucleotide in the sample. From this data, one can also derive information about the physiological source from which the target polynucleotide sample was derived, such as the types of genes expressed in the tissue or cell which is the physiological source of the target, as well as the levels of expression of each gene, particularly in quantitative terms.

Kits

In one embodiment, the present invention provides kits comprising the control nucleic acid molecules described above. Such kits will at least provide one or more control PCR products derived from the control nucleic acid molecules as described above and one or more control mRNA molecules prepared as described above, which may or may not include a polyA-tail. In addition, the kits of the present invention may further comprise additional control nucleic acid molecules in addition to the control nucleic acid molecules. In one embodiment, the present invention provides a kit comprising the following components: (1) 10 μg, lyophilized, of one or more control PCR products generated using the control sequences of SEQ ID Nos 1, 3, 5, 7, 9, 11, 13, 15, 17, or 19 as template; (2) 100 ng (10 ng/μl) of one or more control mRNA molecules transcribed from the control sequences of SEQ ID Nos 2, 4, 6, 8, 10, 12, 14, 16, 18, or 20; (3) 10 μg, lyophilized, of human β-actin PCR product; (4) 1 μg, lyophilized, human Cot-1 DNA; (5) 1 μg, lyophilized, salmon sperm DNA; (6) 0.1 μg, lyophilized, polyA (40-60 bases); (7) 5 ml 3× SSC. Kit components (1)-(7) are preferably each packaged in a separate tube or vial, and each individually packaged kit component (1)-(7) are packaged together in a single container using packaging materials known to those of skill in the art. Alternatively, each of kit components (1)-(7) may be packaged separately in seven separate containers.

Using Control Nucleic Acid to Validate Nucleic Acid Analysis

In one embodiment the control nucleic acid (both PCR products and cDNA molecules) of the present invention may be used to validate an assay comprising nucleic acid hybridization. As used herein, “validate” or “validation” refers to a process by which the measurement of hybridization or lack thereof of a probe nucleic acid to a target nucleic acid is deemed to be accurate. The control nucleic acid molecules described herein can be used to “validate” a number of different aspects of nucleic acid analysis including, but not limited to validating microarray analysis, serving as positive or negative controls, validating mRNA quality, validating differences in dye incorporation and quantum yield, validating expected dye ratios, validating signal linearity and sensitivity of the assay, validation of hybridization consistency within a microarray, validation of RNA isolation techniques, and validation of quantitative PCR.

Positive Controls

In one embodiment, the control nucleic acid molecules are used to “validate” microarray data by serving as positive or negative control samples. When used as a positive control, the control mRNA molecules generated as described above are reverse transcribed and labeled in the same reaction as the experimental or test mRNA. Following the labeling reaction, the control cDNA is hybridized to the control PCR products on the microarray. If a hybridization signal is detected for the control DNA spot, then this indicates that the reverse transcription and labeling reaction worked properly, and that the hybridization reaction was successful. Thus, the accuracy of the hybridization signal or lack thereof of the test samples is thereby “validated”, that is, the lack of a hybridization signal from the test samples indicates either that the appropriate test sequence was not present, or that the test nucleic acids did not have sufficient homology with the target nucleic acid to hybridize under the conditions used. The presence of a hybridization signal from the microarray position containing the control PCR product, thus “validates” the microarray analysis.

Negative Controls

In one embodiment, control DNA/cDNA hybridization is used to “validate” a microarray assay by serving as a negative control. When used as a negative control, the control mRNA is not added to the labeling reaction with the experimental or test mRNA. In the absence of the labeled control cDNA, there should be little or no detectable hybridization signal where the control PCR products were spotted on the microarray. Absence of a detectable hybridization signal from the control PCR spots in this embodiment, would serve to “validate” the microarray analysis, in that, this indicates that there is not a significant level of background hybridization.

Validating mRNA Quality

The quality of the experimental mRNA is critical for successful labeled cDNA preparation. The presence of contaminants, such as cellular carbohydrates and proteins, can cause a decrease in labeling efficiency and an increase in background hybridization signal.

The quality of the experimental mRNA can be determined by quantitating the hybridization signals of human β-actin and positive control spots. Labeled human β-actin cDNA is synthesized from experimental human mRNA whereas control cDNA is synthesized from the control mRNA provided in the kits of the present invention. Detection of hybridization signals from both the human β-actin and positive control spots indicates that the experimental human mRNA is of high quality, that the cDNA was efficiently labeled, and that the hybridization was successful; thereby “validating” the microarray analysis. If significant hybridization signals are detected from only the positive control spots, then the quality of the experimental mRNA is poor. If hybridization signals are not detected from either the human β-actin or control control spots, then one or more parts of the assay (such as the cDNA synthesis/labeling or hybridization) failed. A common cause is when the experimental mRNA contains one or more contaminants, such as RNases, that affected synthesis of the experimental and control cDNA.

Validating Based on Differences in Dye Incorporation and Quantum Yield

It is well-known that Cy3 and Cy5 fluorescent dyes (Amersham Pharmacia Biotech), the most commonly used dyes incorporated into cDNA for use with microarrays, are incorporated at different levels in reverse transcription reactions and have different quantum yields (Worley et al., 2000 Microarray Biochip Technology Eaton Publishing, Mass.). This results in a difference in the Cy3 and Cy5 fluorescence intensities even when equal amounts of Cy3- and Cy5-labeled cDNA are present. These differences can be normalized by (1) determining the ratios of the hybridization signal of equal amounts of the Cy3- and Cy5-labeled control cDNA and then (2) multiplying the values from test or reference cDNA by these ratios. The ratios representing the relative expression levels in the test and reference (i.e., control) mRNA are calculated after data normalization. Normalizing the data prior to calculating the expression ratios for the test DNA allows for comparisons to be made between different experiments and between different laboratories. Thus, when a microarray is normalized as described herein, it is “validated” with respect to the dye properties of the labeled cDNA.

Validating Based on Expected Dye Ratios

Because the expression ratio of the spotted test gene is used to determine if the gene is differentially expressed, it is valuable to be able to determine how the expression ratio correlates with the amount of RNA template added to the labeling reaction. The expected dye ratios are determined by simply adding different amounts of the control mRNA to different dye labeling reactions. For example, add 0.5 and 1.0 nanograms of control mRNA 1 to a Cy3 and CyS labeling reaction, respectively, and compare the hybridization signals following hybridization. The dynamic range of the expression ratios can be determined by creating a standard curve. So determining the expression ratios “validates” the microarray with respect to dye ratios.

Signal Linearity and Sensitivity of the Assay

The labeled control cDNA and spotted DNA are used to determine the signal linearity and sensitivity of the assay. To determine the signal linearity, different amounts of control mRNA are added to test or reference mRNA prior to the cDNA synthesis/labeling reaction. For example, amounts are chosen that correspond to RNA of high, medium, and low abundances. The relative hybridization signals of the control cDNA when hybridized to the corresponding control DNA on the microarray are used to determine the signal linearity. Generating a measurement of the relative hybridization signals of the control cDNA “validates” the microarray analysis with respect to signal linearity.

To determine the sensitivity of the assay, the control mRNA are added to the cDNA-labeling reaction in decreasing amounts. The sensitivity of the microarray assay is indicated as the lowest amount of control cDNA detected. Measurement of the lowest amount of control cDNA detected “validates” the microarray analysis.

Hybridization Consistency Within a Microarray

The consistency of the hybridization signals from different areas of the microarray is a primary concern during the evaluation of microarray data. Factors that can affect the accurate determination of hybridization signals include adequate mixing of the hybridization solution, poor or inconsistent binding of spotted DNA to the slide surface, missing DNA spots, a dirty coverslip, inconsistent or inadequate hybridization temperature, and defects in the microarray surface such as cracks or scratches in the slide coating. The control and controls can be used to identify defective areas within a microarray that should be excluded from further analysis prior to evaluating the overall variation within a microarray using statistics. The number of the control and human β-actin control spots that must be printed is governed by the type of statistical analysis and the desired confidence limits.

Comparing the hybridization signal of each spot for each type of control can identify defective areas in a microarray that should be excluded from analysis. The hybridization signals of all the spots of each type of control should be similar. The presence of an individual control spot with a hybridization signal that deviates significantly from the norm indicates that the control spot and the experimental spots in its vicinity should be examined to determine whether their hybridization signals can be accurately determined or whether the spots should be excluded from further analysis.

The hybridization consistency of each microarray assay is determined statistically by calculating the average variation of replicates of spotted genes (standard deviation of spot values/mean). The average variation of replicates indicates the amount of variation between multiple spots of the same control DNA. In general, an average variation of replicates of<30% indicates a hybridization consistency that is acceptable. Additional statistical methods for determining experimental variation are available from scientific literature. Statistical determination of hybridization consistency thus “validates” the microarray analysis.

The above disclosure generally describes the present invention. A more complete understanding can be obtained by reference to the following specific examples, which are provided herein for purposes of illustration only and are not intended to limit the scope of the invention.

Validating RNA Isolation

In one embodiment, the control nucleic acid molecules of the present invention may be used to validate an RNA isolation procedure. One critical factor in the analysis of cellular nucleic acid expression is the yield of RNA, preferably mRNA, obtained from a cell. In one embodiment, cells to be examined for the expression of a given RNA sequence are mixed under suitable conditions (e.g., in an RNase free aqueous solution such as Trizol) with a known quantity of control nucleic acid (i.e., control mRNA produced as described above) prior to isolation of RNA from the cells. The RNA is subsequently isolated from the cells using techniques known to those of skill in the art (see for example, Ausubel et al., supra). The RNA sample obtained from the cells is thus, mixed with the known quantity of control mRNA. Following isolation, the total RNA sample (cellular RNA+control mRNA) may be analyzed to determine the amount of control mRNA remaining. In one embodiment, the control mRNA is detectably labeled, such that the amount of control mRNA present may be measured by, for example, separating the RNA sample by gel electrophoresis and quantitating the detectable label, wherein the amount of detectable label is indicative of the amount of control mRNA. Alternatively the total RNA sample may be hybridized with a control nucleic acid which is complementary to said control mRNA and is further detectably labeled. The detectable label may then be quantitated, wherein the amount of label detected is indicative of the quantity of control mRNA present in the total RNA sample. By this method, any amount of control mRNA that is lost in the RNA isolation procedure is indicative of the amount of cellular RNA that is lost; the RNA isolation procedure is thus, validated.

Alternatively, varying concentrations of control mRNA may be added to the RNA isolation reaction so as to generate a standard curve, against which the amount of isolated cellular RNA may be evaluated so as to determine the cellular RNA yield.

Validating a Quantitative PCR Assay

In one embodiment, the control nucleic acid molecules of the present invention can be used to validate a TaqMan assay (i.e., real-time PCR). This method is similar to the method described above for using a control mRNA molecule to validate an RNA isolation method. In this embodiment, a known quantity of control mRNA is included in a sample of one or more cells prior to RNA isolation, such that the isolated cellular RNA also includes the control mRNA as described above. Alternatively, the control mRNA may be added to the cellular RNA sample following isolation of the cellular RNA. The total RNA sample (control mRNA+cellular RNA) is then used in a TaqMan assay to quantitate the amount of RNA isolated from the cell sample, wherein the control mRNA is used to generate the standard curve, thus validating the TaqMan assay. TaqMan assays and real-time quantitative PCR techniques are known to those of skill in the art and may be found in, for example U.S. Pat. Nos. 5,691,146; 5,779,977; 5,866,336; and 5,914,230.

In a further embodiment, the control nucleic acid molecules may be labeled with fluor and quencher moieties so as to generate a “control molecular beacon”, useful in, for example, quantitative PCR assays. A “control molecular beacon” comprises a hairpin, or stem-loop structure which possesses a pair of interactive signal generating labeled moieties (e.g., a fluorophore and a quencher) effectively positioned to quench the generation of a detectable signal when the beacon is not hybridized to the test nucleic acid sequence. The loop comprises a region that is complementary to a test nucleic acid (i.e., control nucleic acid complementary to the control molecular beacon). The loop is flanked by 5′ and 3′ regions (“arms”) that reversibly interact with one another by means of complementary nucleic acid sequences when the region of the probe that is complementary to a nucleic acid target sequence is not bound to the target nucleic acid. Alternatively, the loop is flanked by 5′ and 3′ regions (“arms”) that reversibly interact with one another by means of attached members of an affinity pair to form a secondary structure when the region of the probe that is complementary to a nucleic acid target sequence is not bound to the target nucleic acid. As used herein, “arms” refers to regions of a control molecular beacon probe that a) reversibly interact with one another by means of complementary nucleic acid sequences when the region of the molecular beacon that is complementary to a nucleic acid test sequence is not bound to the test nucleic acid or b) regions of a beacon that reversibly interact with one another by means of attached members of an affinity pair to form a secondary structure when the region of the beacon that is complementary to a nucleic acid test sequence is not bound to the test nucleic acid. When a molecular beacon is not hybridized to test sequence, the arms hybridize with one another to form a stem hybrid, which is sometimes referred to as the “stem duplex”. This is the closed conformation. When a molecular beacon hybridizes to the test nucleic acid, the “arms” of the beacon are separated. This is the open conformation. In the open conformation an arm may also hybridize to the test nucleic acid. Such beacons may be free in solution, or they may be tethered to a solid surface. When the arms are hybridized (e.g., form a stem) the quencher is very close to the fluorophore and effectively quenches or suppresses its fluorescence, rendering the beacon dark. Such molecular beacon molecules are described in U.S. Pat. No. 5,925,517 and U.S. Pat. No. 6,037,130, and these teachings may be adapted by one of skill in the art to the control nucleic acid molecules of the present invention to generate “control molecular beacons”. The invention encompasses molecular beacon probes wherein one or more subunits of the beacon comprise a molecular beacon structure.

A wide range of fluorophores may be used in control molecular beacons according to this invention. Available fluorophores include coumarin, fluorescein, tetrachlorofluorescein, hexachlorofluorescein, Lucifer yellow, rhodamine, BODIPY, tetramethylrhodarmine, Cy3, Cy5, Cy7, eosine, Texas red and ROX. Combination fluorophores such as fluorescein-rhodamine dimers, described, for example, by Lee et al. (1997), Nucleic Acids Research 25:2816, are also suitable. Fluorophores may be chosen to absorb and emit in the visible spectrum or outside the visible spectrum, such as in the ultraviolet or infrared ranges.

Suitable quenchers described in the art include particularly DABCYL and variants thereof, such as DABSYL, DABMI and Methyl Red. Fluorophores can also be used as quenchers, because they tend to quench fluorescence when touching certain other fluorophores. Preferred quenchers are either chromophores such as DABCYL or malachite green, or fluorophores that do not fluoresce in the detection range when the beacon is in the open conformation.

The control molecular beacon molecules may be incorporated, along with known amounts the complementary control nucleic acid molecule, into a quantitative PCR reaction, whereby quantification of the amount of complementary control nucleic acid molecule detected by the control molecular beacon molecules validates the quantitative PCR reaction.

### EXAMPLES

The examples below are non-limiting and are merely representative of various aspects and features of the present invention.

### Example 1

### Generation of Control Nucleic Acid Molecules

Ten 500-nucleotide control DNAs were designed using a PHP4 script program running on a desktop Linux 6.2 computer. A total of 260 sequences were designed and include ten members for each group of different GC-content (20%, 25%, . . . 75%, 80%). The ten sequences with a 50% GC-content were used to construct the control nucleic acid molecules of SEQ ID Nos 1-20.

The design algorithm included six general steps. First, a “random” sequence of a given length with desired GC-content was generated as described in the preceding paragraph. Second, the sequence was checked for the presence of long stretches of low-complexity sequences (mono-, di-, tri- and tetranucleotides), and if such sequences were absent then this sequence was accepted. Third, the newly accepted sequence was subjected to multiple cycles of random cleavage in multiple positions, following by shuffling and recombination of the resulting subfragments. Then the second step was repeated, and if the sequence passed the filters then it was accepted. Fourth, the process of iterative cleavage/shuffling/filtering was continued until the number of accepted sequences for each GC-content group reached ten. Fifth, the process started from the first step for the next GC-content group. In order to exclude similar sequences which might lead to cross-hybridization, the multiple BLAST procedure was performed for the entire pool of 260 designed sequences. The matches were considered significant at the 96% identity over>50 bases of alignable sequence. No matches were found at these conditions. In addition, BLAST analysis against non-redundant database (nr) was performed at random for the sets of sequences within GC-content 45-55%, and again, no matches longer than 13 base pairs were found.

Construction of Control DNA

The 500-bp control DNA sequences of SEQ ID Nos 1-20 were constructed from overlapping oligonucleotides in 2 separate extension reactions followed by six sequential PCR to direct the non-template addition of sequences to each end of the DNA generated in the previous reaction (FIG. 1). The extension reaction conditions were: 2.5 U Taq2000, 200 μM each dNTP and 100 pmol each oligonucleotide in 1× cloned Taq buffer in a 50-ul reaction. The oligonucleotide name, reaction description, reaction number, oligonucleotide name and nucleotide sequence are given in Table 1. The extension products were analyzed by agarose gel electrophoresis.

Equimolar amounts of the 2 extension reactions were combined and used as the template in the first series of PCR. The PCR conditions were: 2.5 U Taq2000, 200 μM each dNTP and 100 pmol each oligonucleotides in 1× cloned Taq buffer in a 50-μl reaction. Thirty cycles of 93° C. for 0.5 min, 55° C. for 0.5 min, and 72° C. for 1 min; and 1 cycle of 72° C. for 10 min. After the first 3 rounds of PCR, the extension time was increased from 1 min to 1.5 min. The PCR products were analyzed by agarose gel electrophoresis. The PCR product from each PCR was used as the template in the next PCR. An additional PCR was performed with control DNA inserts 1-5 and 7-8 using an additional set of oligonucleotide primers to reverse the cloning sites. The PCR products were purified using the PCR High Pure Kit (Roche) prior to restriction digestion.

A 25-bp polyA tail was added to each control DNA in a seventh PCR. The PCR conditions were: 2.5 U TaqPlus Precision, 0.2 mM each dNTP and 100 pmol each oligonucleotide in 1× TaqPlus Precision buffer in a 50-μl reaction. Thirty cycles of 93° C. for 0.5 min, 55° C. for 0.5 min, and 72° C. for 1.5 min; and 1 cycle of 72° C. for 10 min. The PCR products were analyzed by agarose gel electrophoresis. The PCR products were purified using the PCR High Pure Kit (Roche) prior to restriction digestion.

The lack of homology between the control nucleic acid sequences of SEQ ID Nos 1-20 and known nucleic acids was demonstrated by comparing the control nucleic acid to sequences in the GeneConnection Discovery Clone Collection (www2.stratagene.com) and NIH genetic databases (Altschul et al., 1997 Nucleic Acids Research 25: 3389). The results of these comparisons are shown in Table 4 (an “x” indicates that no significant homology was identified to any sequence in the particular database). In addition, fluorescence-labeled human HeLa cDNA did not hybridize to the control PCR products spotted on arrays (shown below). Also, the control nucleic acid molecules were compared to each other by BLAST analysis and do not have homology to each other. cDNA generated from these genes are therefore unlikely to hybridize to DNA from any organism or cross hybridize to each other making these genes useful in any microarray system.

TABLE 4

| | BAS | BAS | BAS | BAS | BAS | BAS | BAS | BAS | BAS | BAS |
|---|---|---|---|---|---|---|---|---|---|---|
| | 50001 | 50002 | 50003 | 50004 | 50005 | 50006 | 50007 | 50008 | 50009 | 500010 |
| NCBI web site | | | | | | | | | | |
| nr | x | x | x | x | x | x | x | x | x | x |
| Drosophila genome | x | x | x | x | x | x | x | x | x | x |
| month | x | x | x | x | x | x | x | x | x | x |
| dbest | x | x | x | x | x | x | x | x | x | x |
| dbsts | x | x | x | x | x | x | x | x | x | x |
| mouse ests | x | x | x | x | x | x | x | x | x | x |
| human ests | x | x | x | x | x | x | x | x | x | x |
| other ests | x | x | x | x | x | x | x | x | x | x |
| pdb | x | x | x | x | x | x | x | x | x | x |
| kabat | x | x | x | x | x | x | x | x | x | x |
| mito | x | x | x | x | x | x | x | x | x | x |
| alu | x | x | x | x | x | x | x | x | x | x |
| epd | x | x | x | x | x | x | x | x | x | x |
| yeast | x | x | x | x | x | x | x | x | x | x |
| E. coli | x | x | x | x | x | x | x | x | x | x |
| gss | x | x | x | x | x | x | x | x | x | x |
| GC web site | | | | | | | | | | |
| HGS | x | x | x | x | x | x | x | x | x | x |
| htgs | x | x | x | x | x | x | x | x | x | x |
| GC | x | x | x | x | x | x | x | x | x | x |
| nt | x | x | x | x | x | x | x | x | x | x |
| cds_human | x | x | x | x | x | x | x | x | x | x |
| cds_mouse | x | x | x | x | x | x | x | x | x | x |
| patnt | x | x | x | x | x | x | x | x | x | x |
| vector | x | x | x | x | x | x | x | x | x | x |
| est_human nr | x | x | x | x | x | x | x | x | x | x |
| est_mouse nr | x | x | x | x | x | x | x | x | x | x |
| est_nr | x | x | x | x | x | x | x | x | x | x |
| Hs.seq.all | x | x | x | x | x | x | x | x | x | x |
| Hs.seq.unique | x | x | x | x | x | x | x | x | x | x |
| Mm.seq.all | x | x | x | x | x | x | x | x | x | x |
| Mm.seq.unique | x | x | x | x | x | x | x | x | x | x |
| yeast.nt | x | x | x | x | x | x | x | x | x | x |
| ecoli.nt | x | x | x | x | x | x | x | x | x | x |
| sts | x | x | x | x | x | x | x | x | x | x |
| alu.n | x | x | x | x | x | x | x | x | x | x |

### Example 2

### Generation of Control PCR Products and Labeled Control cDNA

Construction of Plasmids for Preparing PCR Products

The PCR products without the polyA tail and pBluescript II SK+ were digested with 40 U EcoR I in 1.5× Universal buffer 37° C. for 1 hour and purified with the PCR High Pure Kit (Roche). The EcoR I-digested PCR products and pBluescript II SK+ were digested with 10 U Xho I in 1× Universal buffer at 37° C. for 1 hour and purified as described above prior to ligation.

The insert (control nucleic acid SEQ ID Nos 1, 3, 5, 7, 9, 11, 13, 15, 17, 19) and vector were combined in a 3:1 molar ratio and ligated at 14° C. for 5 hours using the DNA Ligation Kit. XL10-Gold competent cells (kanr) were transformed with the ligated DNA using standard conditions and plated on Luria Broth containing 50 μg/ml ampicillin. Isolated colonies were screened for the presence of insert by PCR using 5′ insert- (Table 2) and 3′ vector- (5′-TGAGCGGATAACAATTTCACACAG-3′; SEQ ID NO: 205) specific primers using the same PCR conditions given above to add the 25-bp polyA tail. DNA was isolated from colonies containing plasmids with the desired insert with a maxiprep kit (Qiagen, Valencia, Calif.). The identity of each clone and the presence of the cloning sites were verified by determining the nucleotide sequence of the cDNA insert on both strands using the dye terminator method (ABI, Foster City, Calif.).

Construction of Plasmids for Preparing RNA

The PCR products with the polyA tail (i.e., SEQ ID Nos 2, 4, 6, 8, 10, 12, 14, 16, 18, 20) and pBluescript II KS+ were digested with EcoR I and Xho I, ligated, the correct constructs identified, and the nucleotide sequence determined as described above in “Construction of plasmids for preparing PCR products”. The only change in the protocol is that when the colonies were screened to identify plasmids containing the insert, the 3′ vector-specific primer was 5′-GTTTTCCCAGTCACGACGTTG-3′ (SEQ ID NO: 206).

Characterization of Plasmids

The control plasmids can be distinguished from each other by restriction digestion. However, since some of the restriction digestion products are relatively small, the most reliable methods of distinguishing between the plasmids are by PCR with insert-specific primers (Table 2) followed by restriction digestion at the unique site (Table 3) or by determining the nucleotide sequence.

Preparation of Control PCR Products

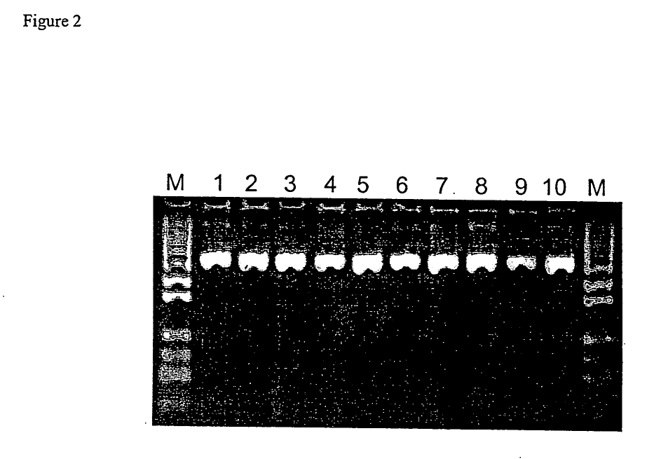

PCR products of each control DNA and human beta-actin were prepared as follows. The PCR conditions were: 2.5 U TaqPlus Precision, 200 μM each DNTP and 100 pmol of the 5′ and 3′ PCR primer (Table 2) in 1× TaqPlus Precision buffer in a 100-ul reaction. Thirty cycles of 93° C. for 0.5 min, 55° C. for 0.5 min, and 72° C. for 1.5 min; and 1 cycle of 72° C. for 10 min. The PCR products were analyzed by agarose gel electrophoresis and purified by ethanol precipitation with sodium acetate (FIG. 2). The concentration of the resuspended PCR products was determined by using picogreen (Molecular Probes) and a FluorTracker (Stratagene). DNA yields were 8-36 μg from each 100 μl PCR reaction with is higher than expected (Table 5).

TABLE 5

| Control DNA | DNA yield (ug) |
|---|---|
| 1 | 26 |
| 2 | 20 |
| 3 | 36 |
| 4 | 22 |
| 5 | 22 |
| 6 | 25 |
| 7 | 31 |
| 8 | 20 |
| 9 | 8 |
| 10 | 11 |

Preparation of Control mRNA

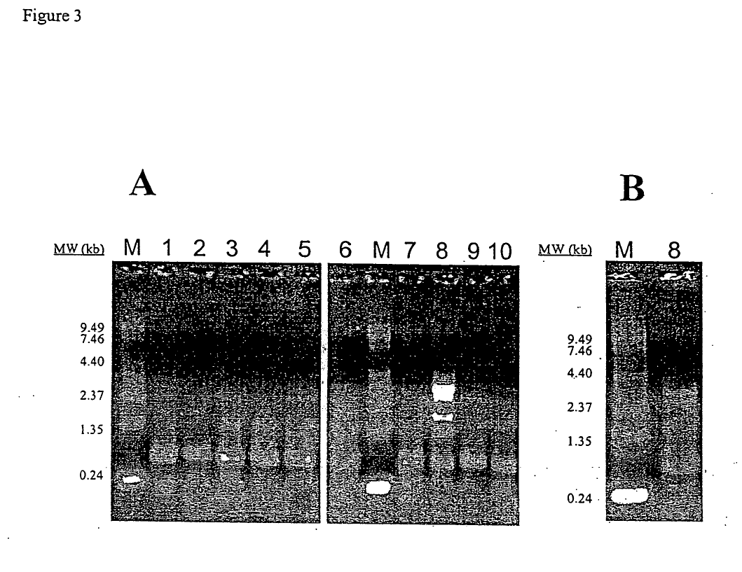

Polyadenylated control mRNA was prepared by in vitro transcription using the plasmids with inserts having polyA tails. The transcription protocol is described in detail in the SpotReport-10 array validation kit (Stratagene). For these experiments, the reaction was scaled down and contained 2.5 ug of each linearized plasmid for each transcription reaction. The transcription reactions were performed twice. The quantity and quality of the mRNA was determined by measuring the absorption at 260 and 280 nanometers (nm) and by denaturing agarose gel electrophoresis (FIG. 3). The OD 260/280 and RNA yields are given in Table 6. The RNA from the first transcription had a significant amount of lower molecular weight nucleic acid visible on the gel in most of the samples (data not shown). This was probably due to incomplete digestion of the plasmid DNA. The presence of this nucleic acid did not appear to effect the mRNA function, however, since DNA also adsorbs at 260 nm, it did effect the RNA quantitation. If this nucleic acid is present in future production lots of the mRNA, the RNA should be treated with DNase and purified until it is removed. The RNase-free DNase used to digest the DNA in the first RNA transcription was from the StrataPrep RNA Miniprep isolation kit (Stratagene). The DNase used to digest the DNA in the second RNA transcription was the stand-alone RNase-free DNase (Stratagene; cat no 600031). Based on these results, it is preferred to use the stand alone RNase-free DNase.

The OD 260/280 ratio was used to determine the amount and quality of the RNA. Preferably, the OD 260/280 ratio for RNA is 1.8-2.0. In these experiments, the ratios ranged from 1.6 to 2.4 in the first transcription and 1.0 to 1.8 in the second transcription. Although these ratios are not ideal, the ratios did not seem to effect our ability to label the mRNA. The ratio of 1.0 is from an RNA sample with the lowest RNA concentration and may therefore not be accurate. RNA yields ranged from 3 to 55 μg from 2.5 μg of linearized plasmid in the first transcription and 6 to 32 from 2.5 μg of linearized plasmid in the second transcription (Table 6). The yields and OD 260/280 were more consistent in the second than in the first transcription. The first transcriptions were performed at different times with different sets and combinations of reagents and may have contributed to the inconsistencies in these numbers.

TABLE 6

| | First transcription | | Second transcription | |
|---|---|---|---|---|
| | | mRNA yield (ug) | | mRNA yield (ug) |
| | | per 2.5 ug | | per 2.5 ug |
| Control | OD | of linearized | OD | of linearized |
| DNA | 260/280 | plasmid | 260/280 | plasmid |
| 1 | 1.9 | 55 | 1.54 | 32 |
| 2 | 2.0 | 3 | 1.05 | 6 |
| 3 | 2.3 | 6 | 1.69 | 24 |
| 4 | 1.6 | 11 | 1.76 | 25 |
| 5 | 2.0 | 16 | 1.84 | 26 |
| 6 | 1.7 | 30 | 1.85 | 20 |
| 7 | 2.3 | 30 | 1.65 | 23 |
| 8 | 1.7 | 10 | 1.64 | 14 |
| 9 | 2.4 | 7 | 1.69 | 26 |
| 10 | 2.3 | 30 | 1.59 | 18 |

More than one RNA species was generated by in vitro transcription from plasmid 8A. At first, this was thought to be from incomplete digestion with EcoR I when linearizing the plasmid prior to transcription. However, repeated digestions with EcoR I and other enzymes with recognition sites adjacent to the EcoR I site were not successful in completely digesting this plasmid. An alternative explanation is that this plasmid prep contained more than one plasmid. For this reason, the construction and characterization of the plasmid containing control 8 insert with polyA was repeated.

Preparation of Labeled Control cDNA

Fluoresence-labeled cDNA was prepared by adding 25 picograms (pg) of each control mRNA to 10 ug HeLa total RNA and converting it to Cy3- or Cy5-labeled cDNA using the FiarPlay labeling kit (Stratagene). In some experiments, 50 pg of each A. thaliana mRNA (SpotReport-10array validation kit, Stratagene) was also added. In one experiment, no control mRNA was added to the HeLa total RNA. The labeled cDNA was purified using the spin columns provided in the kit and analyzed by agarose gel electrophoresis as follows. A thin agarose gel was prepared by pouring 2% (w/v) agarose gel in 1× TAE buffer on a 2 cm×3 cm glass microscope slide. 0.5 ul of each sample was loaded onto the gel and electrophoresed at 125 volts (V) for 0.5 hour. The Cy-3 labeled cDNA was visualized using a 2 color, laser/PMT Prototype Microarray Scanner (John Parker; UCLA). Cy3 was detected with a PMT using a 532 nm laser with 580 nm-emission filter and Cy5 was detected with a PMT using a 635 nm laser with 700 nm-emission filter.

### Example 3

### Preparation of Control DNA Arrays

Arrays were created by spotting control DNA PCR products, human Cot-1 DNA, salmon sperm DNA, polyA (40-60 bases) and 3× SSC onto poly L lysine-coated slides. The PCR products, human Cot-1 and salmon sperm DNA were spotted at a DNA concentration of 0.1 ug/ul in 3× SSC and the polyA (40-60 bases) at a concentration of 0.01 ug/ul in 3× SSC. The DNA were spotted onto poly L lysine-coated slides with a Gene Machines arrayer using a standard protocol with 2 minor modifications. A 100 millisecond contact time and an extended wash program were used to ensure a minimum amount of DNA carryover. The microarrays were processed after spotting according to our standard blocking procedure (see Microarray Labeling kit manual, Stratagene; cat. no. 252001).

A second set of arrays was created as described above. This set of arrays also included A. thaliana PCR products (SpotReport-10, cat no 252010), A. thaliana oligonucleotides (70-mers) and control oligonucleotides (70-mers). The oligonucleotides were spotted at a concentration of 40 uM. The contact time was decreased from 100 to 50 milliseconds. Four slide surfaces were compared by spotting poly L lysine-coated slides, CMT-GAP II slides (Corning), SuperAmine slides (Telechem) and dendrimer slides (Haoqiang Huang; Stratagene). Five different DNA spotting solutions were used to spot the DNA on these slide surfaces. The DNA spotting solutions were 3× SSC, 50% DMSO, 5% sodium bicarbonate, 50% DMSO in 0.1× TE and 3× SSC, 1.5M betaine. Nonspecific DNA binding sites were blocked following the slide manufacturer's recommended protocols.

### Example 4

### Hybridization and Detection of Labeled Control cDNA

The fluorescence-labeled cDNA was hybridized to a microarray using standard methods (Microarray Labeling Kit manual, Stratagene; cat. no. 252001). In each experiment, ⅙ of the total labeling reaction of each dye was used. Hybridization was detected with the Axon GenePix 4000 scanner and data analyzed with the Axon GenePix Pro analysis software (Axon Instruments, Union City, Calif.) following the manufacturer's recommended protocols.

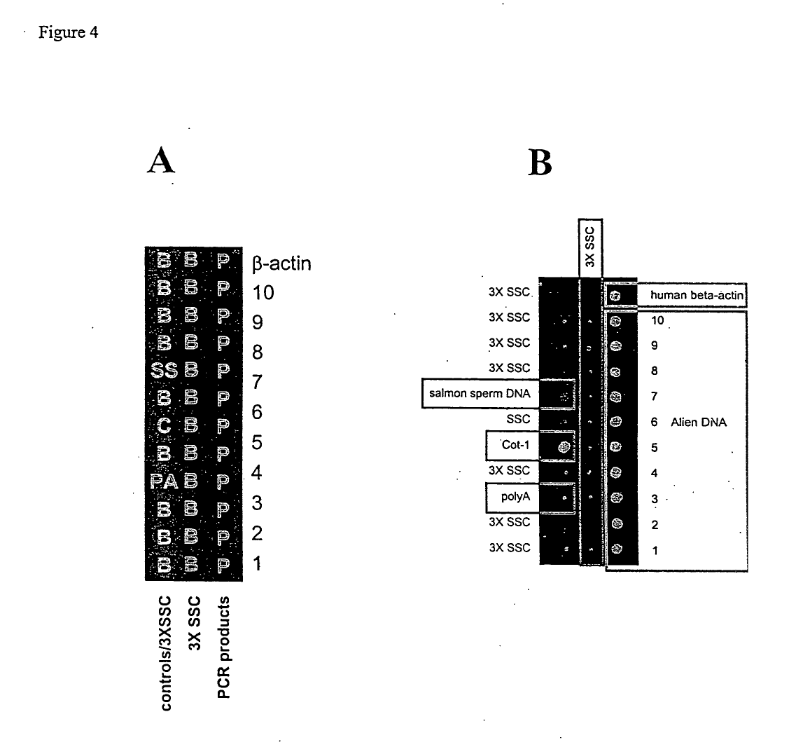

Fluorescence-labeled control, A. thaliana and/or HeLa cDNA were hybridized to arrays (FIGS. 4, 5 and 6). As expected, the fluorescence-labeled control cDNA hybridized strongly to the control PCR products spotted on the array. And the fluorescence-labeled human beta-actin hybridizes to the beta-actin spotted on the array. The fluorescence-labeled cDNA does not hybridize to the spotted 3× SSC, salmon sperm DNA or polyA but does hybridize to the spotted human Cot-1 DNA (Cot-1). This is because salmon sperm and polyA DNA are included as blocking reagents in the hybridization buffer but human Cot-1 DNA is not. There is strong hybridization to Cot-1 because human Cot-1 DNA is highly enriched for repetitive sequences and the fluorescence-labeled cDNA includes repetitive sequences.

Fluorescence-labeled control and HeLa cDNA were hybridized to spotted control PCR products to verify that the labeled control cDNA hybridized to the spotted control PCR products. FIG. 4A shows the spotting pattern for the 3× SSC (B); control PCR product (P); salmon sperm DNA (SS); human Cot-1 DNA (C); and polyA (PA). The results clearly indicate that in the presence of labeled control cDNA, there is hybridization to the spotted control DNA (FIG. 4B). In this experiment, the fluorescence-labeled HeLa hybridized to the beta-actin PCR product and to the human Cot-1 DNA. Beta-actin is highly expressed in HeLa, therefore, labeled beta-actin strongly hybridizes to the spotted beta-actin PCR product. The labeled HeLa hybridized to the human Cot-1 DNA because HeLa is a human cell line and many of the human RNA in this cell line contain the repetitive sequences found in Cot-1. Human Cot-1 is generally included as a blocking reagent in blocking buffers, however, it was not included in this buffer.

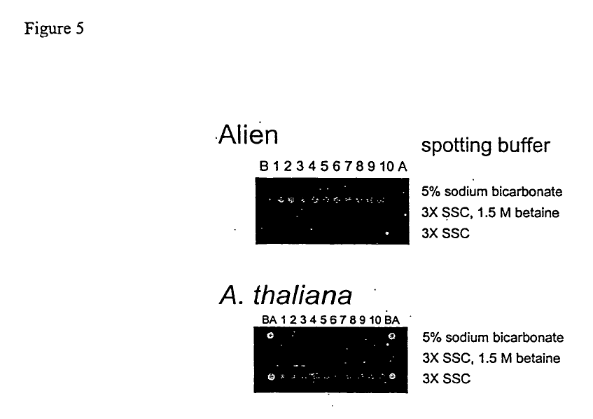

Fluorescence-labeled human HeLa cDNA was hybridized to spotted control PCR products to verify that mRNA expressed in human HeLa cells does not hybridize to the control DNA. The results clearly indicate that in the absence of labeled control cDNA, there is no hybridization to either the control or A. thaliana PCR products by the labeled HeLa cDNA (FIG. 5). Due to expression of beta-actin in HeLa cells, the labeled HeLa cDNA hybridized to the beta-actin PCR products. These results demonstrate that the labeled human HeLa cDNA does not hybridize to the spotted control PCR products.

Spotting Buffer and Slide Surface Comparisons

The most commonly used slide surface is a poly L lysine-coated slide. While there are many other surfaces available, most users continue to use poly L lysine-coated slides because of their low cost and the lack of a significant advantage of other slide surfaces. However, some users will want to spot on other commercially available slide surfaces. We therefore spotted the control PCR products on slides that were amine-modified (SuperAmine, Telechem), dendrimer-coated (Haoqiang Huang; Stratagene) and amino-silane coated (CMT-GAP™ II coated slides, Corning). Nonspecific binding to the slides was blocked following each of the manufacturer's protocols. The same Cy-labeled control and HeLa cDNA was hybridized to the slides and the slides were all processed at the same time under the same conditions.

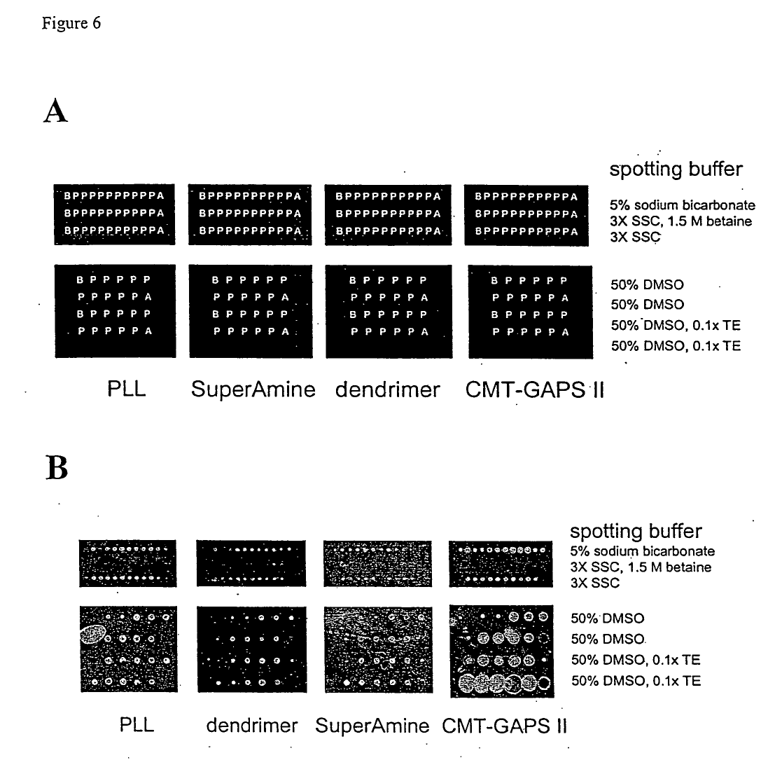

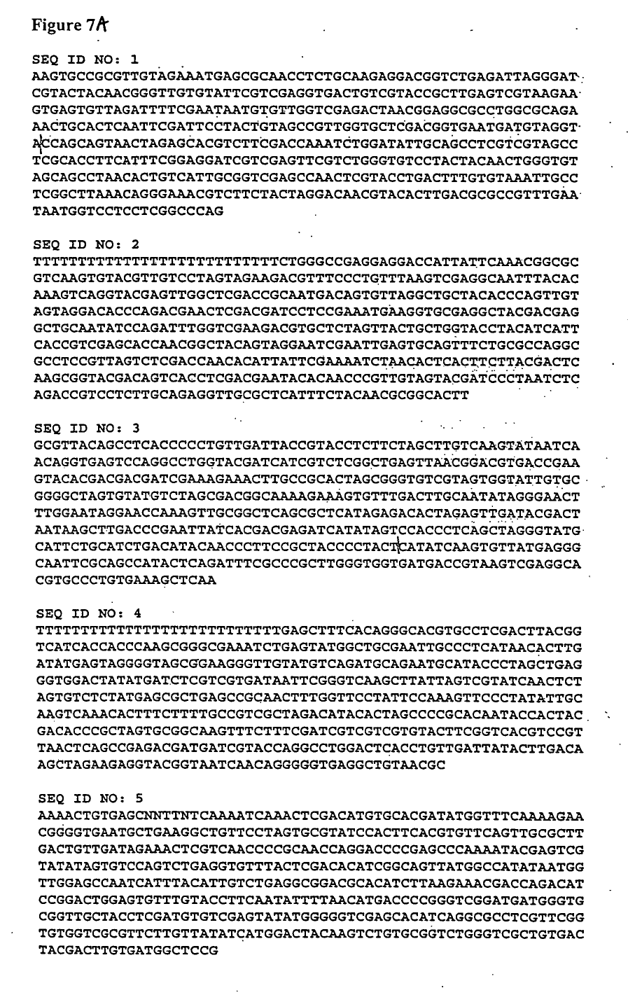

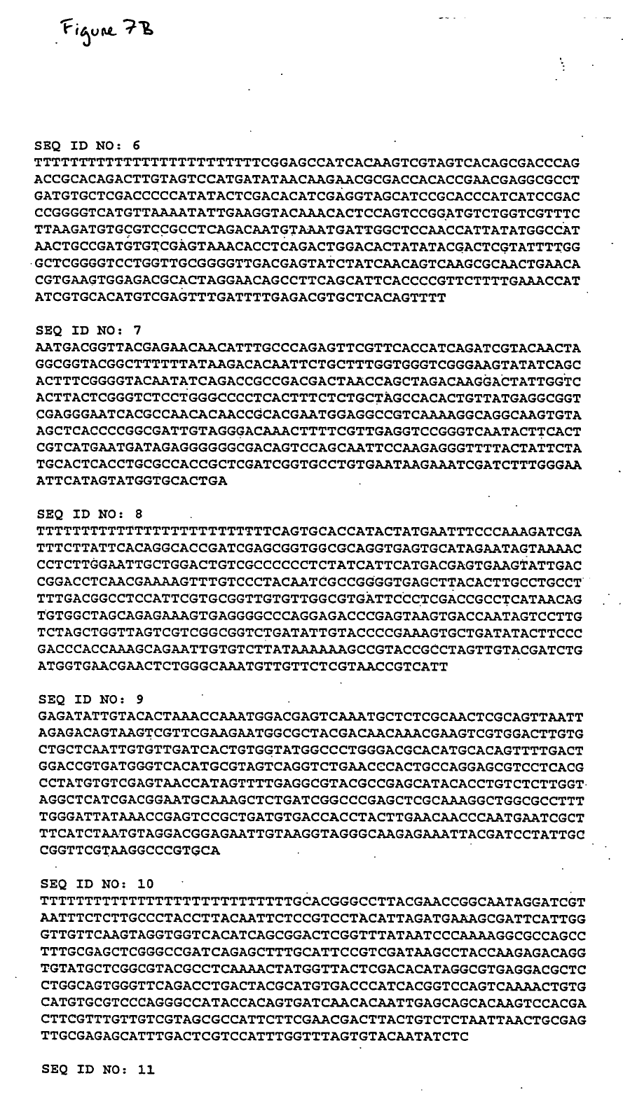

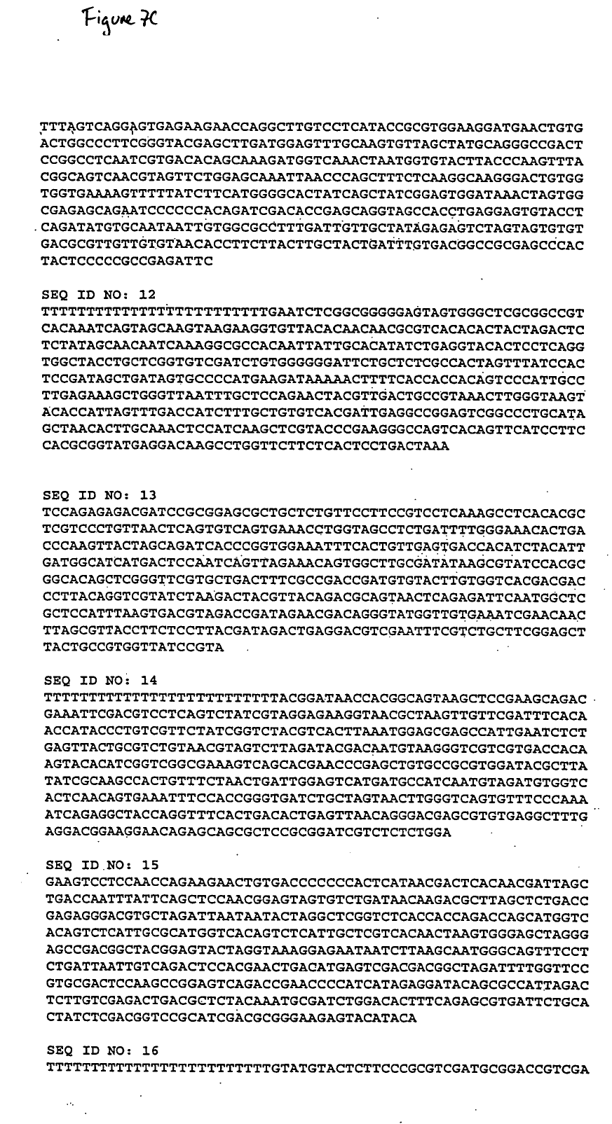

FIG. 6A shows the spotting pattern used for 3× SSC (B); control PCR products (P); and polyA (A); the control PCR products are spotted 1 to 10 from left to right. The spotting buffers and slide surfaces were evaluated for spot size consistency and hybridization signal intensity (FIG. 6B). The spotting buffer with the most consistent spot size and hybridization intensity on the poly L lysine-coated slides was 3× SSC. The hybridization signal was higher from the DMSO spots than from the 3× SSC spots but the spot size was inconsistent. Inconsistencies in spot sizes can increase the amount of time and effort required for data analysis and is therefore undesirable. Further optimization would be required to improve the spot size consistency when spotting with DMSO. The preferred combinations of printing buffer and slide surface are shown in Table 7. The other slide surfaces were similarly evaluated and recommended spotting buffers identified (Table 5). These results are consistent with the spotting buffers recommended by each manufacturer. In subsequent experiments, the background on the SuperAmine slides was similar to that of poly L lysine slides. The cause of the high background on this slide is not due to the labeled cDNA since the same cDNA did not produce high background on the other slides. The cause of this high background is not known.

TABLE 7

| | | | | | 50% |
|---|---|---|---|---|---|
| | 5% sodium | 3X SSC, 1.5 | | 50% | DMSO, |
| slide surface | bicarbonate | M betaine | 3X SSC | DMSO | 0.1x TE |
| poly L | | | x | | x |
| lysine | | | | | |
| dendrimer | x | | | x | x |
| SuperAmine | | | | | x |
| CMT GAPS | | | x | | |
| II | | | | | |

TABLE 8

Exemplary Useful Fragments of Control Nucleic Acids of the
Invention
Control DNA fragment sequence (5′ to 3′)

| | | |
|---|---|---|
| SEQ ID NO: 207 | CCAGCAGTAACTAGAGCACGTCTTCGACCAAATCTGGATATTGCAGCCTCG | Nucleotides 242-311 of |
| | TCGTAGCCTCGCACCTTCA | SEQ ID NO: 1 |
| SEQ ID NO: 208 | CATATCAAGTGTTATGAGGGCAATTCGCAGCCATACTCAGATTTCGCCCGC | Nucleotides 401-470 of |
| | TTGGGTGGTGATGACCGTA | SEQ ID NO: 3 |
| SEQ ID NO: 209 | GCGCCTCGTTCGGTGTGGTCGCGTTCTTGTTATATCATGGACTACAAGTCT | Nucleotides 408-477 of |
| | GTGCGGTCTGGGTCGCTGT | SEQ ID NO: 5 |
| SEQ ID NO: 210 | CGGTCGAGGGAATCACGCCAACACAACCGCACGAATGGAGGCCGTCAAAAG | Nucleotides 237-306 of |
| | GCAGGCAAGTGTAAGCTCA | SEQ ID NO: 7 |
| SEQ ID NO: 211 | ACATGCGTAGTCAGGTCTGAACCCACTGCCAGGAGCGTCCTCACGCCTATG | Nucleotides 196-266 of |
| | TGTCGAGTAACCATAGTTT | SEQ ID NO: 9 |
| SEQ ID NO: 212 | CTTGTCCTCATACCGCGTGGAAGGATGAACTGTGACTGGCCCTTCGGGTAC | Nucleotides 27-96 of |
| | GAGCTTGATGGAGTTTGCA | SEQ ID NO: 11 |
| SEQ ID NO: 213 | CATGACTCCAATCAGTTAGAAACAGTGGCTTGCGATATAAGCGTATCCACG | Nucleotides 189-158 of |
| | CGGCACAGCTCGGGTTCGT | SEQ ID NO: 13 |
| SEQ ID NO: 214 | CCAATTTATTCAGCTCCAACGGAGTAGTGTCTGATAACAAGACGCTTAGCT | Nucleotides 64-133 of |
| | CTGACCGAGAGGG | SEQ ID NO: 15 |
| SEQ ID NO: 215 | AACAGTATGTGTCACAAACGTACCAGCTCTGCCTAAATCCGGCCAAGTCGC | Nucleotides 68-137 of |
| | TTTAGCACCTCATGTGAGC | SEQ ID NO: 17 |
| SEQ ID NO: 216 | CCCCGAATCAGGAACATGCGTCCTCTAAGAACTTTAGGTGACCATCAGCGT | Nucleotides 135-204 of |
| | AGCATACCAACTCCTTGAC | SEQ ID NO: 19 |

### Other Embodiments

The foregoing examples demonstrate experiments performed and contemplated by the present inventors in making and carrying out the invention. It is believed that these examples include a disclosure of techniques which serve to both apprise the art of the practice of the invention and to demonstrate its usefulness. It will be appreciated by those of skill in the art that the techniques and embodiments disclosed herein are preferred embodiments only that in general numerous equivalent methods and techniques may be employed to achieve the same result.

All of the references identified hereinabove are hereby expressly incorporated herein by reference to the extent that they describe, set forth, provide a basis for or enable compositions and/or methods which may be important to the practice of one or more embodiments of the present invention.

1. A method for validating a hybridization reaction comprising
(a) synthesizing a nucleic acid complement of a plurality of RNA molecules comprising mRNAs and at least one control probe nucleic acid molecule, wherein said plurality of RNA molecules are templates for said synthesizing, and wherein said synthesizing is performed in the presence of a primer capable of priming nucleic acid synthesis from said mRNAs and said control probe nucleic acid molecule;
(b) hybridizing the nucleic acid synthesized in (a) to a collection of target nucleic acid molecules, wherein at least one molecule of said collection is complementary to the nucleic acid synthesized from said control probe nucleic acid;
(c) detecting said nucleic acid complement of said at least one control nucleic acid hybridized to a nucleic acid molecule of said collection.

2. The method of claim 1, wherein said synthesizing is further performed in the presence of an enzyme which synthesizes nucleic acid from said templates.

3. The method of claim 1, wherein nucleic acid not specifically hybridized to said collection is removed from the hybridization reaction.

4. The method of claim 1, wherein nucleic acid not specifically hybridized to said collection is removed from the hybridization reaction under high stringency conditions.

5. The method of claim 1, wherein said control probe nucleic acid is control mRNA or DNA.

6. The method of claim 1, wherein said synthesizing step (b) further comprises one or more dNTPs which are detectably labeled.

7. The method of claim 6, wherein said detectable label is a fluorescent label.

8. The method of claim 1 wherein said at least one molecule of said collection complementary to said nucleic acid synthesized from said control probe nucleic acid does not hybridize to the complement of an adenine-rich region in said nucleic acid synthesized from said control probe nucleic acid.

9. A method of making a control target nucleic acid comprising:
(a) linking a control nucleic acid molecule to a nucleic acid vector to form a recombinant nucleic acid construct;
(b) introducing said construct into a host cell;
(c) growing said host cell under conditions which permit replication of said construct
(d) isolating said construct from said host cell; and
(e) synthesizing a nucleic acid complement of said construct wherein said synthesizing is performed in the presence of (i) one or more primers capable of priming nucleic acid synthesis from said construct and (ii) an enzyme which synthesizes nucleic acid from said construct.

10. The method of claim 9, wherein said enzyme is DNA polymerase.

11. A method of making a control probe nucleic acid comprising
(a) linking a control nucleic acid molecule to a nucleic acid vector to from a recombinant nucleic acid construct;
(b) introducing said construct into a host cell;
(c) growing said host cell under conditions which permit replication of said construct,
(d) isolating said construct from said host cell;
(e) synthesizing an mRNA copy of said construct wherein said synthesizing is performed in the presence of a first enzyme which synthesizes mRNA from said construct; and
(f) synthesizing a nucleic acid complement of said mRNA wherein said synthesizing is performed in the presence of (i) one or more primers capable of priming nucleic acid synthesis from said mRNA and (ii) a second enzyme which synthesizes nucleic acid from said mRNA.

12. The method of claim 11, wherein said nucleic acid complement is a cDNA.

13. The method of claim 11, wherein said nucleic acid complement is detectably labeled.

14. The method of claim 11, wherein said first enzyme is RNA polymerase.

15. The method of claim 11, wherein said second enzyme is reverse transcriptase.

16. A method of using a control target nucleic acid comprising:
(a) immobilizing said control target nucleic acid on a solid support;
(b) hybridizing said control target with a control probe nucleic acid; and
(c) detecting said control probe nucleic acid hybridized to said control target nucleic acid.

17. The method of claim 16, wherein said control probe nucleic acid is detectably labeled.

18. The method of claim 16 wherein said solid support is a solid surface.

19. A method of making a control nucleic acid comprising the steps of:
(a) synthesizing a nucleic acid molecule with a random sequence and having a preselected G/C-content to produce a synthetic nucleic acid molecule;
(b) comparing said nucleic acid molecule with a database of nucleic acid molecules, wherein if a nucleic acid molecule contained in said database is not at least 5% identical to said synthetic nucleic acid molecule said method proceeds to step (c).
(c) synthesizing a single nucleic acid complement of said synthetic nucleic acid wherein said synthesizing is performed in the presence of i) a first primer capable of priming said synthesis from said synthetic nucleic acid molecule and ii) an enzyme which synthesizes DNA from said synthetic nucleic acid;
(d) synthesizing two or more nucleic acid complements of said synthetic nucleic acid wherein said synthesizing is performed in the presence of i) a second primer capable of priming synthesis from said single nucleic acid complement synthesized in step (c) or a set of such primers, and ii) an enzyme which synthesizes nucleic acid from said synthetic nucleic acid;
(e) repeating step (d) one to seven times, each time in the presence of a different second primer or set of different second primers, whereby said repeating said synthesizing generates a control nucleic acid molecule.

20. The method of claim 19 wherein said second primer or set of second primers comprises a 3′-terminal region of 12-30 nt that are complementary to the 3′ 12-30 nt of a strand of said single nucleic acid complement synthesized in step (c).

21. The method of claim 32, wherein in step (e), each different second primer or set of different second primers comprises a 3′ terminal region of 12-30 nt that are complementary to the 3′ 12-30 nucleotides of a product of the previous performance of step (d).

22. The method of claim 19 further comprising the step, after step(a), of discarding all synthetic nucleic acid molecules of step (a) that comprise more than 5 contiguous G nucleotides, more than 5 contiguous C nucleotides, more than 6 contiguous A nucleotides, more than 6 contiguous T nucleotides, or more than 3 tandem repeats of any di-, tri-, or tetranucleotide sequence.

23. The method of claim 21 wherein step (a) further comprises the steps of:
(i) generating 20 nucleotides of nucleic acid sequence, wherein said sequence has a 50% G/C content and wherein said sequence further comprises fewer than 6 contiguous G nucleotides, fewer than 6 contiguous C nucleotides, fewer than 7 contiguous A nucleotides, fewer than 7 contiguous T nucleotides, and fewer than 4 tandem repeats of any di-, tri-, or tetranucleotide sequence;
(ii) cleaving the 20 nucleotide nucleic acid sequence at least two times at random positions; and
(iii) ligating the cleaved sequences to produce a ligated sequence that is different from that of the nucleic acid sequence generated in step (a), and wherein the ligated sequence comprises fewer than 6 contiguous G nucleotides, fewer than 6 contiguous C nucleotides, fewer than 7 contiguous A nucleotides, fewer than 7 contiguous T nucleotides, and fewer than 4 tandem repeats of any di-, tri-, or tetranucleotide sequence.

24. The method of claim 19, wherein said step (d) is a PCR reaction.

25. The method of claim 19, wherein said enzyme is a DNA polymerase.

26. A method of using a control nucleic acid comprising:
(a) mixing a known amount of said control nucleic acid with one or more non-control nucleic acid molecules;
(b) detecting said control nucleic acid.

27. The method of claim 26, wherein said control nucleic acid is detectably labeled.

28. A method of using a control nucleic acid comprising:
(a) mixing a known amount of said control nucleic acid with one or more isolated RNA molecules;
(b) synthesizing two or more copies of said control nucleic acid and said one or more isolated RNA molecules, wherein said synthesizing is performed in the presence of i) primers capable of priming said synthesis from said control nucleic acid molecule and said one or more isolated RNA molecules and ii) an enzyme which synthesizes nucleic acid from said control nucleic acid and said one or more isolated RNA molecules; and
(c) detecting said control nucleic acid.

29. The method of claim 28, wherein said control nucleic acid is detectably labeled.

30. An isolated synthetic nucleic acid molecule of at least 40 nucleotides in length, having less than 5% homology to any known nucleic acid sequence naturally found in a living organism, and having 20% to 80% G/C content, wherein said synthetic nucleic acid does not hybridize over a region of at least 30 contiguous nucleotides under high stringency conditions to any nucleic acid molecule other than its own complement, and wherein said synthetic nucleic acid comprises fewer than 6 contiguous G nucleotides, fewer than 6 contiguous C nucleotides, fewer than 7 contiguous A nucleotides, fewer than 7 contiguous T nucleotides, and fewer than 4 tandem repeats of any di-, tri-, or tetranucleotide sequence.

31. The synthetic nucleic acid molecule of claim 30 which substantially lacks secondary structure.

32. An isolated nucleic acid molecule that is the complement of the synthetic nucleic acid molecule of claim 30.

33. The nucleic acid molecule of claim 30 or the complement thereof, said molecule further comprising a 3′ adenine-rich region of 10 to 200 nucleotides or the complement thereof.

34. The isolated synthetic molecule of claim 30, further comprising a detectable marker.

35. The molecule of claim 34, wherein said detectable marker comprises a fluorescent moiety.

36. A vector comprising a nucleic acid molecule of claim 30.

37. A host cell comprising a vector of claim 36.

38. An isolated synthetic nucleic acid molecule of any one of SEQ ID NOs: 1-20 or a fragment thereof comprising at least 40 nucleotides, or the complement of said molecule or fragment thereof.

39. An isolated synthetic nucleic acid molecule comprising a sequence selected from the group consisting of: nucleotides 242-311 of SEQ ID NO: 1; nucleotides 401-470 of SEQ ID NO: 3; nucleotides 408-477 of SEQ ID NO: 5; nucleotides 237-306 of SEQ ID NO: 7; nucleotides 196-266 of SEQ ID NO: 9; nucleotides 27-96 of SEQ ID NO: 11; nucleotides 189-158 of SEQ ID NO: 13; nucleotides 64-133 of SEQ ID NO: 15; nucleotides 68-137 of SEQ ID NO: 17; nucleotides 135-204 of SEQ ID NO: 19; and the complement of any of these.

40. An isolated synthetic nucleic acid molecule selected from the group consisting of: nucleotides 242-311 of SEQ ID NO: 1; nucleotides 401-470 of SEQ ID NO: 3; nucleotides 408-477 of SEQ ID NO: 5; nucleotides 237-306 of SEQ ID NO: 7; nucleotides 196-266 of SEQ ID NO: 9; nucleotides 27-96 of SEQ ID NO: 11; nucleotides 189-158 of SEQ ID NO: 13; nucleotides 64-133 of SEQ ID NO: 15; nucleotides 68-137 of SEQ ID NO: 17; nucleotides 135-204 of SEQ ID NO: 19; and the complement of any of these.

41. The isolated synthetic molecule of any one of claims 38-40, said molecule further comprising a detectable marker.

42. The molecule of claim 41, wherein said detectable marker comprises a fluorescent moiety.

43. A vector comprising a nucleic acid molecule of any one of claims 38-40.

44. A host cell comprising a vector of claim 43.

45. An isolated synthetic nucleic acid having 50% G/C content and lacking greater than 5% homology to any known naturally-occurring nucleic acid sequence, said nucleic acid selected from the group consisting of SEQ ID Nos. 21-22, 38-39, 55-56, 72-73, 89-90, 106-107, 121-122, 138-139, 155-156, and 169-170, or a fragment thereof comprising at least 40 nucleotides of a said nucleic acid.

46. A collection of nucleic acid molecules comprising a plurality of target nucleic acids and at least one control target nucleic acid molecule complementary to a control probe nucleic acid.

47. A collection of nucleic acid molecules comprising a plurality of target nucleic acids and at least one control target molecule complementary to a control probe nucleic acid comprising an adenine-rich region of 10 to 200 nucleotides, wherein said at least one control target nucleic acid molecule complementary to said control probe nucleic acid is not complementary to said adenine rich region of said control probe nucleic acid.

48. The collection of claim 46 or 47, wherein said control probe nucleic acid is cDNA.

49. The collection of claim 46 or 47, wherein said control probe nucleic acid is an RNA.

50. The collection of claim 46 or 47, wherein said collection is immobilized on a solid substrate.

51. The collection of claim 50, wherein said solid substrate is a solid surface.

52. A hybrid nucleic acid molecule comprising a control target nucleic acid molecule hybridized to a control probe nucleic acid molecule.

53. The hybrid nucleic acid molecule of claim 52, wherein said control target nucleic acid molecule is immobilized on a solid surface.

54. A kit containing
(a) a control probe RNA molecule;
(b) a control target nucleic acid molecule complementary to said control probe RNA molecule; and
(c) packaging materials therefor.

55. A kit containing
(a) a control probe RNA molecule containing an adenine-rich region of 10 to 200 nucleotides;
(b) a control target nucleic acid molecule complementary to said control probe RNA but lacking the adenine-rich region; and
(c) packaging materials therefor.

56. The kit of claim 54 or 55, wherein said control target nucleic acid is DNA.

57. The kit of claim 54 or 55, further comprising an enzyme which synthesizes DNA from said control RNA probe.

58. The synthetic nucleic acid molecule of claim 30, wherein said nucleic acid molecule has a sequence selected from the group consisting of the sequence of SEQ ID Nos 1-20.
